(19) United States
(12) Patent Application Publication
(10) Pub. No.: US 2025/0288686 A1
(43) Pub. Date: Sep. 18, 2025

(54) MULTIMERIC PROTEIN-ALBUMIN CONJUGATE, PREPARATION METHOD THEREFOR, AND USE THEREOF

(71) Applicant: ProAbtech Co., LTD., Gwangju (KR)

(72) Inventors: Jeong Haeng CHO, Gyeonggi-do (KR); Sun Oh Shin, Gwangju (KR); Hyun Woo Kim, Seoul (KR); Byungseop Yang, Seoul (KR); Min Hyung Kim, Seoul (KR); Seong Ju Park, Gwangju (KR)

(21) Appl. No.: 18/274,670

(22) PCT Filed: Jan. 28, 2022
(86) PCT No.: PCT/KR2022/001675

§ 371 Date: Jul. 27, 2023

(30) Foreign Application Priority Data
Jan. 29, 2021 (KR) 10-2021-0013537
Jul. 8, 2021 (KR) 10-2021-0089516
Sep. 23, 2021 (KR) 10-2021-0125723

(51) Int. Cl. A61K 47/64; A61K 47/65

(52) U.S. Cl. CPC A61K 47/643; A61K 47/65

(57) ABSTRACT

Disclosed herein is a multimeric protein-albumin conjugate. The multimeric protein-albumin conjugate has a multimeric protein variant conjugated with albumin via a linker. In the multimeric protein-albumin conjugate, a certain number of albumins are conjugated via a linker to a multimeric protein variant that has an amino acid sequence properly modified to form a conjugate in a site-specific manner. The multimeric protein variant results from substitution of a non-natural amino acid for one or more amino acids in the amino acid sequence of the corresponding wild-type multimeric protein, wherein the linker and albumin is linked to the non-natural amino acid residue.

### CROSS-REFERENCE TO RELATED APPLICATIONS

This application is a national stage filing under 35 U.S.C. § 371 of PCT International Application No. PCT/KR2022/001675, filed Jan. 28, 2022, which claims the benefit of application No. 10-2021-0013537 (KR), filed Jan. 29, 2021; application No. 10-2021-0089516 (KR), filed Jul. 8, 2021; and application No. 10-2021-0125723 (KR), filed Sep. 23, 2021 in Korea. The entire contents of PCT/KR2022/001675 are incorporated herein in their entirety by this reference.

### INCORPORATION BY REFERENCE OF MATERIAL SUBMITTED ELECTRONICALLY

The instant application contains a Sequence Listing which has been filed electronically in ASCII format and is hereby incorporated by reference in its entirety. Said ASCII copy, created on Feb. 27, 2024, is named P220126US_ST25_Revised.txt and is 336,718 bytes in size.

### TECHNICAL FIELD

In the present disclosure, disclosed are a technology for site-specifically introducing a nonnatural amino acid into a multimeric protein, a technology for conjugating albumin to the multimeric protein, into which the nonnatural amino acid is introduced, through a linker, a multimeric protein-albumin conjugate itself, and use thereof.

### BACKGROUND ART

Therapeutic protein has been reported to be effective in the treatment of various diseases, and it is one of the important growth motives in the pharmaceutical industry. However, there is a problem in that therapeutic protein is continuously removed by glomerular filtration, pinocytosis, and immune response in a patient's body. Therefore, when developing a therapeutic protein, it is very important to extend the duration of the drug effect by lowering the rate at which it is removed from the patient's body due to such a phenomenon. A technique for improving the half-life to solve the problem by physically or chemically binding albumin to a therapeutic protein is called albumination.

On the other hand, an inverse electron-demand Diels-Alder reaction (IEDDA reaction) is one type of click chemistry, which is characterized by being modular, having a wide application range, having a high yield, generating insignificant byproducts, being stereospecific, being physiologically stable, having a large thermodynamic driving force (for example, 84 kJ/mol or more), and having a high atom economy.

The inventors of the present disclosure completed a site-specific albumination technology universally applicable to various multimeric proteins using the inverse electron-demand Diels-Alder reaction (IEDDA reaction).

### DISCLOSURE

### Technical Problem

The present disclosure aims to provide a multimeric protein-albumin conjugate.

The present disclosure aims to provide a method of preparing the multimeric protein-albumin conjugate.

The present disclosure aims to provide a multimeric protein variant contained in the multimeric protein-albumin conjugate.

The present disclosure aims to provide a method of preparing the multimeric protein variant.

The present disclosure aims to provide a pharmaceutical composition containing the multimeric protein-albumin conjugate.

The present disclosure aims to provide use of the multimeric protein-albumin conjugate.

### Technical Solution

Provided Herein is a Tetramer Protein-Albumin Conjugate Represented by [Formula 1]:

T-[J₁-A-J₂-HSA]ₙ [formula 1]
wherein the T is a tetramer protein variant, J₁ is a tetramer protein-linker junction, A is an anchor, J₂ is an albumin-linker junction, and HSA is Human Serum Albumin,
wherein n is 3 or 4,
the tetramer protein variant is a tetramer that four of variant subunits are oligomerized,
each of the variant subunit comprises one 4-(1,2,3,4-tetrazine-3-yl) phenylalanines (frTet), whereby the tetramer protein variant comprises four frTet,
the tetramer protein-linker junction has a junction structure formed through Inverse Electron Demand Diels-Alder (IEDDA) reaction between a tetrazine moiety of frTet of the tetramer protein and trans-cyclooctene moiety linked to the anchor,
the tetramer protein-linker junction is represented by following,

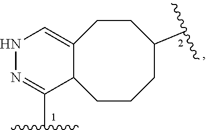 [SMILES: *C1=NNC=C2CCC(*)CCCC21]

wherein the (1) is linked to the residue of the nonnatural amino acid, and the (2) is linked to the anchor,
wherein the albumin-linker junction is a junction structure formed through a reaction between a thiol moiety of the albumin and a thiol reactive moiety of the anchor.

In one embodiment, an amino acid sequence of the albumin is characterized in that it is selected from a group consisting of SEQ ID NO: 47 to 57.

In one embodiment, the albumin-linker junction has a junction structure of which thiol group of 34th cysteine of the albumin and thiol reactive moiety of the anchor are bounded,
wherein the junction structure of the albumin-linker junction is selected from the following:

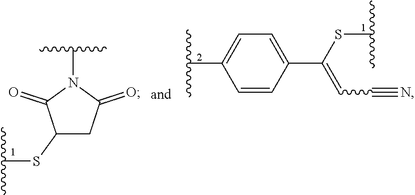

wherein, (1) is linked to the albumin, and (2) is linked to the anchor.

In one embodiment, the anchor is characterized in that it is selected from the following:

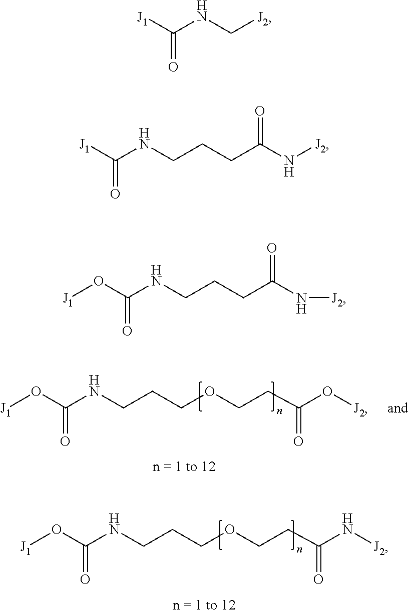

wherein J₁ is tetramer protein-linker junction, J₂ is albumin-linker junction.

In one embodiment, an amino acid sequence of the variant subunit of the tetramer protein variant is characterized in that it is selected from a group consisting of SEQ ID NOs: 4 to 17, wherein the X of the amino acid sequence is frTet.

In one embodiment, an amino acid sequence of the variant subunit of the tetramer protein variant is characterized in that it is selected from a group consisting of SEQ ID NOs: 18 to 40, and 138, wherein the X of the amino acid sequence is frTet.

In one embodiment, an amino acid sequence of the variant subunit of the tetramer protein variant is characterized in that it is selected from a group consisting of SEQ ID NOs: 41 to 45, and 139 to 158, wherein the X of the amino acid sequence is frTet.

Provided Herein is a Method for Manufacturing a Tetramer Protein-Albumin Conjugate, the Method Comprises:
reacting an albumin and a linker,
wherein the linker comprises a dienophile functional group, an anchor, and a thiol reactive moiety,
wherein the dienophile functional group is a trans-cyclooctene or a derivative of trans-cyclooctene,
wherein the thiol reactive moiety is selected from a maleimide or a derivative of maleimide, and a 3-arylpropiolonitriles or a derivative of 3-arylpropiolonitriles,
wherein the thiol reactive moiety of the linker is bound with thiol moiety of albumin through reaction to form an albumin-linker conjugate; and
reacting the albumin-linker conjugate and a tetramer protein variant,
wherein, the tetramer protein variant is a tetramer that four of variant subunits are oligomerized,
wherein each of the variant subunit comprises one 4-(1,2,3,4-tetrazine-3-yl) phenylalanines (frTet), whereby the tetramer protein comprises four frTet,
wherein the tetrazine functional group of the frTet and the is bound with the dienophile functional group of the linker through Inverse Electron Demand Diels-Alder (IEDDA) reaction to form a tetramer protein-albumin conjugate,
wherein the tetramer protein-albumin conjugate is characterized in that three or more albumins are conjugated to the tetramer protein variant through the linkers.

In one embodiment, the linker is characterized in that it is selected from followings:

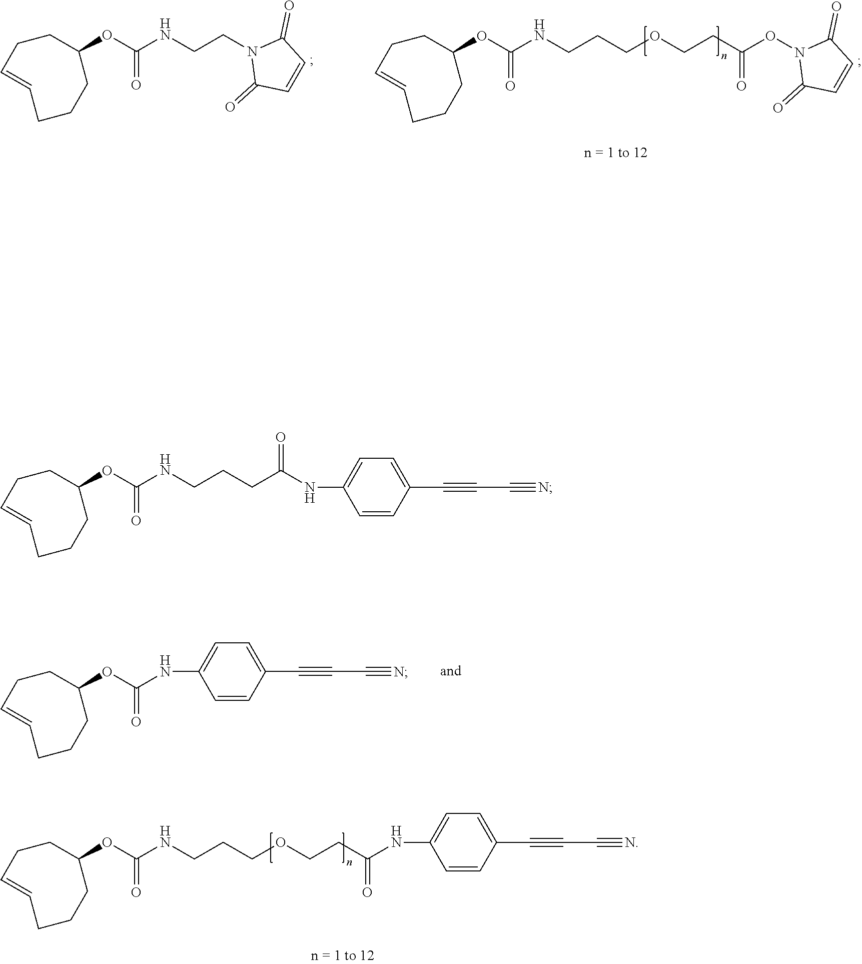

In one embodiment, it is characterized in that the albumin is represented by an amino acid sequence selected from a group consisting of SEQ ID NOs: 47 to 57, wherein the thiol reactive moiety of the linker and the thiol group of 34th cysteine of the albumin are bounded through reaction.

In one embodiment, it is characterized in that an amino acid sequence of the variant subunit of the tetramer protein variant is selected from a group consisting of SEQ ID NOs: 4 to 17, wherein the X of the amino acid sequence is frTet.

In one embodiment, it is characterized in that an amino acid sequence of the variant subunit of the tetramer protein variant is selected from a group consisting of SEQ ID NOs: 18 to 40, and 138, wherein the X of the amino acid sequence is frTet.

In one embodiment, it is characterized in that an amino acid sequence of the variant subunit of the tetramer protein variant is selected from a group consisting of SEQ ID NOs: 41 to 45, and 139 to 158, wherein the X of the amino acid sequence is frTet.

Provided Herein is a Method for Manufacturing a Tetramer Protein-Albumin Conjugate, the Method Comprises:
disrupting a cell,
wherein, the cell comprises a tetramer protein variant,
wherein the tetramer protein variant comprises at least one 4-(1,2,3,4-tetrazine-3-yl) phenylalanines (frTet);
adding an albumin-linker conjugate to the cell disruption product,
wherein, the albumin-linker conjugate comprises a trans-cyclooctene or a derivative of trans-cyclooctene as a dienophile functional group,
wherein the tetrazine functional group of the frTet and the is bound with the dienophile functional group of the linker through Inverse Electron Demand Diels-Alder (IEDDA) reaction to form a multimeric protein-albumin conjugate; and
obtaining the multimeric protein-albumin conjugate.

In one embodiment, it is characterized in that the method further comprises pretreatment process before adding an albumin-linker conjugate to the cell disruption product, wherein the cell disruption product is generated as a result of the pretreatment process.

In one embodiment, it is characterized in that the albumin-linker conjugate is represented by [formula 2]:

I-A-J-HSA [formula 2]
wherein, the I is the dienophile functional group, selected from followings,

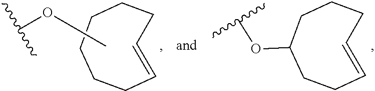

wherein the A is an anchor, selected from followings,

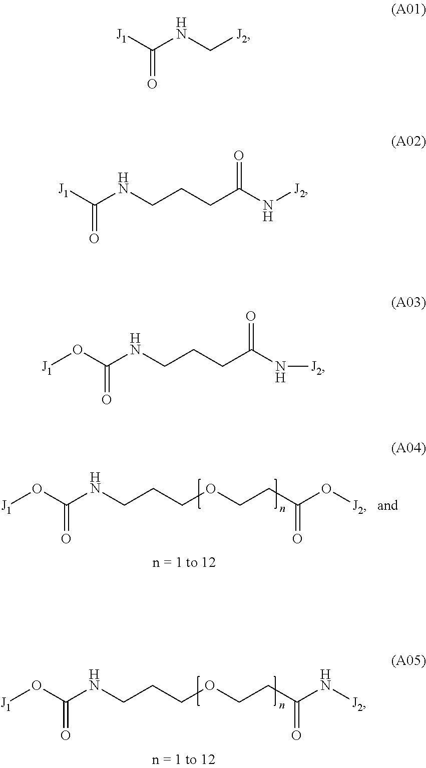

wherein the J is linker-albumin junction, selected from followings,

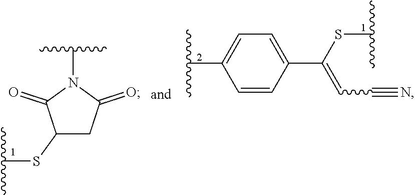 [SMILES: *SC1CC(=O)N(*)C1=O ; *SC(=CC#N)c1ccc(*)cc1]

wherein the (1) is linked to an albumin, and the (2) is linked to the anchor of the linker,
wherein the HSA is the albumin represented by a sequence selected from a group consisting of SEQ ID NOs: 47 to 57.

In one embodiment, it is characterized in that the cell further comprises at least one of the followings:
pDule_C11 disclosed in Yang et.al (Temporal Control of Efficient In Vivo Bioconjugation Using a Genetically Encoded Tetrazine-Mediated Inverse-Electron-Demand Diels?Alder Reaction, Bioconjugate Chemistry, 2020, 2456-2464); tyrosyl-tRNA synthase derived from Methanococcus jannaschii (MjTyrRS); and suppressor tRNA derived from Methanococcus jannaschii (MjtRNATyrCUA).

### Advantageous Effects

According to the technical problems and the solutions thereof disclosed herein, provided is a multimeric protein-albumin conjugate. The multimeric protein-albumin conjugate has an activity equivalent to or superior to that of a wild-type multimeric protein-albumin conjugate and exhibits improved in vivo stability and low immunogenicity, which are advantages of the conjugate prepared through albumination. Therefore, the multimeric protein can exhibit excellent effects when used for therapeutic purposes.

### DESCRIPTION OF DRAWINGS

FIG. 1 illustrates multimeric protein-albumin preparation methods disclosed herein, in which Process 1 illustrates a method of firstly reacting albumin with a linker and Process 2 illustrates a method of firstly reacting a multimeric protein with a linker, where ALB means albumin and M.P means the multimeric protein;

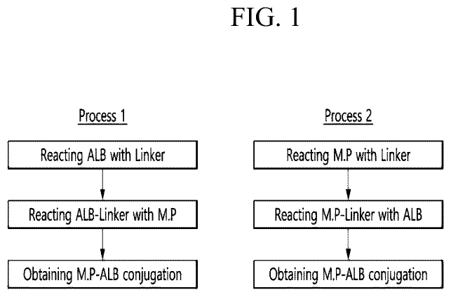

FIG. 2 illustrates flow charts for novel processes among multimeric protein-albumin preparation methods disclosed herein, in which Process A illustrates a conventionally used method and Process B illustrates a novel process, where ALB means albumin and M.P means the multimeric protein;

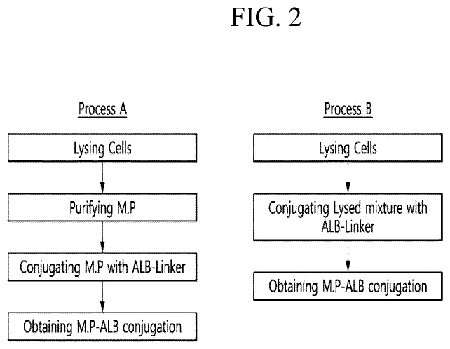

FIG. 3 shows a chromatogram of SP Sepharose Fast Flow purification for a urate oxidase variant in Experimental Example 4;

FIG. 4 shows results of SP Sepharose Fast Flow purification for a urate oxidase variant in Experimental Example 4, where 1 means a marker, and 2 means a purified product of the urate oxidase variant;

FIG. 5 shows an HPLC chromatogram of a resulting product of SP Sepharose Fast Flow purification for a urate oxidase variant in Experimental Example 4;

FIG. 6 shows preparation results of a urate oxidase-albumin conjugate in Experimental Example 4.3, where M means a marker, 1 means the results obtained immediately after Uox lysate-HSA conjugation, and 2 means the results obtained immediately after Uox-HSA conjugation, in which 101 kDa indicates the urate oxidase-albumin conjugate, 67 kDa indicates albumin, and 34 kDa indicate a urate oxidase variant;

FIG. 7 shows preparation results of a urate oxidase-albumin conjugate in Experimental Example 4.4, where M means a marker, 1 means a urate oxidase variant, and 2 means the result obtained immediately after Uox-HSA conjugation, in which 101 kDa indicates the urate oxidase-albumin conjugate, 67 kDa indicates albumin, and 34 kDa indicates a urate oxidase variant;

FIG. 8 shows results of evaluating activities of methioninase-albumin conjugates in Experimental Example 3.5, in which the vertical axis represents methioninase activity, where METase-WT means wild-type methioninase, METase (P148)-rHA means a methioninase-albumin conjugate of SEQ ID NO: 42, and METase (P283)-rHA means a methioninase-albumin conjugate of SEQ ID NO: 45;

FIG. 9 shows PK analysis results of methioninase-albumin conjugates in Experimental Example 3.6, in which the vertical axis represents serum activity and the horizontal axis represents time, where METase-WT means wild-type methioninase, METase (P148)-rHA means a methioninase-albumin conjugate of SEQ ID NO: 42, and METase (P283)-rHA means a methioninase-albumin conjugate of SEQ ID NO: 45, whereupon the half-lives of METase-WT, METase (P148)-rHA, and METase (P283)-rHA appear to be 0.8 hours, 1.4 hours, and 4.9 hours, respectively;

FIG. 10 shows SDS-PAGE analysis results of protein overexpression patterns for 5 types of asparaginase variant strains, where S and IS represent soluble and insoluble proteins, in which D85UAG means an asparaginase variant of SEQ ID NO: 20, Q240UAG means an asparaginase variant of SEQ ID NO: 28, E269UAG means an asparaginase variant of SEQ ID NO: 33, E289UAG means an asparaginase variant of SEQ ID NO: 37, and K319UAG means an asparaginase variant of SEQ ID NO: 39;

FIG. 11 shows Ni-NTA purification and SDS-PAGE analysis results of an asparaginase variant (K319UAG)-6H protein, where M, 1, 2, 3, 4, and 5 represent a marker, soluble, insoluble, FT, washing, and elution, respectively;

FIG. 12 shows a Hitrap IMAC-FF purification profile of asparaginase (K319UAG)-6H;

FIG. 13 shows results of band analysis in fluorescence (left) and SDS-PAGE staining (right) by binding TCO-Cy3 to fractions of asparaginase variant (K319UAG)-6H, the fractions purified using Hitrap IMAC-FF;

FIG. 14 shows SDS-PAGE analysis results of an asparaginase variant (K319UAG)-albumin conjugate mixture, where M means a marker and 1 means the asparaginase-albumin conjugate mixture;

FIG. 15 shows SDS-PAGE analysis results of protein overexpression pattern for 5 types of 6h-methioninase variant strains, where S and IS represent soluble and insoluble proteins, in which A144UAG′ means a methioninase variant of SEQ ID NO: 41, P148UAG means a methioninase variant of SEQ ID NO: 42, K177UAG means a methioninase variant of SEQ ID NO: 43, E283UAG means a methioninase variant of SEQ ID NO: 44, and T300UAG means a methioninase variant of SEQ ID NO: 45;

FIG. 16 shows Ni-NTA purification and SDS-PAGE analysis results of 6h-methioninase variant (P148UAG or P283UAG)-6H protein, where M, 1, 2, 3, 4, and 5 represent a marker, soluble, insoluble, FT, washing, and elution, respectively;

FIG. 17 shows SEC-FPLC purification profiles of 6h-methioninase variants (P148UAG or P283UAG);

FIG. 18 shows SDS-PAGE results of fractions of a 6h-methioninase variant (P283UAG), the fractions purified by SEC-FPLC;

FIG. 19 shows a result of confirming a fluorescence band by binding a TCO-Cy3 linker to 6H-methioninase (P283UAG) after performing Ni-NTA purification, where M, 1, 2, 3, and 4 represent a marker, lysate, FT, washing, and elution, respectively, whereupon a METase band appears to be 42.5 kDa;

FIG. 20 shows SEC-HPLC analysis results of a methioninase-albumin conjugate and methioninase;

FIG. 21 is a diagram in which (a) shows a crystal structure of AgUox (PDB code: 2YZE) showing selected sites for frTet incorporation, and (b) shows a tabulation of the corresponding AgUox variants containing frTet and frTet incorporation sites;

FIG. 22 is a diagram regarding expression and purification of AgUox-frTet variation, in which (a) shows an image of Coomassie blue-stained protein gel of AgUox-WT and AgUox-frTet (Ag1-14) variations, lanes: MW, molecular weight marker; BI, before induction; AI, after induction, and (b) shows an image of Coomassie blue-stained protein gel of AgUox-WT and the AgUox-frTet variants after purification;

FIG. 23 relates to thermal stability evaluation of AgUox-WT and AgUox-frTet variants, in which (a) shows relative enzyme activities of AgUox-WT and the AgUox-frTet (Ag1-14) variants monitored in PBS at 0 and 120 hours, where the red line represents 50% enzyme activity of AgUox-WT, and (b) shows relative enzyme activities of AgUox-WT and the five AgUox-frTet variants (Ag1, Ag6, Ag8, Ag10, and Ag12) monitored at 0, 120, and 240 hours, in which the relative activities of the AgUox-frTet variants were normalized to the enzyme activity of AgUox-WT;

FIG. 24 shows incorporation of frTet into AgUox, showing Coomassie blue-stained protein gel for fluorescence (illumination Aex=302 nm, wavelengths of 510 nm and 610 nm in the Chemidoc XRS+ system) and reaction mixtures of TCO-Cy3 and AgUox-WT or AgUox-frTet (Ag) variants;

FIG. 25 shows SDS-PAGE analysis results of AgUox-HSA conjugate variants, in which protein gel was imaged with Coomassie blue staining, where 1, 2, 3, 4, and 5 represent AgUox-HSA from Ag1 variant, AgUox-HSA from Ag6 variant, AgUox-HSA from Ag8 variant, AgUox-HSA from Ag10 variant, and AgUox-HAS from Ag12 variant, respectively;

FIG. 26 is a diagram regarding purification of HSA-conjugated Ag12 variant (Ag12-HSA), showing SDS-PAGE analysis results of size-exclusion chromatography performed on an Ag12-HSA conjugate mixture (right) and elution fractions of Ag12-HSA (F1-F3) (left), in which protein gel was stained with Coomassie blue for protein band visualization;

FIG. 27 shows relative enzyme activities of AgUox-WT, Ag12, and Ag12-HSA, in which the experiment was performed in triplicate, where error bars represent standard deviations;

FIG. 28 is a diagram regarding pharmacokinetic studies of AgUox-WT and Ag12-HSA in mice, in which enzyme activities of residual AgUox-WT and Ag12-HSA conjugate samples were measured at 15 minutes, in which the samples were intravenously injected into BALB/c female mice (n=4) at 3 hours, 6 hours, and 12 hours for AgUox-WT and at 15 minutes, 12 hours, 24 hours, 48 hours, and 72 hours for Ag12-HAS, where error bars represent standard deviation;

FIG. 29 shows enzyme activities of AfUox-WT and AgUox-WT over time, in which the enzyme activities were confirmed using an enzyme activity assay as described in Experimental Method 6, in which the relative activities of AfUox-WT and AgUox-WT were normalized to the activity of AfUox-WT;

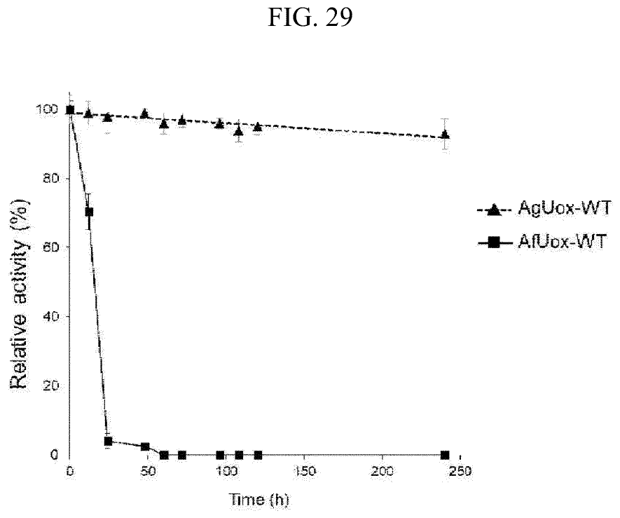

FIG. 30 shows analysis results of matrix-assisted laser desorption/ionization time-of-flight mass spectra (MALDI-TOF MS) for (a) Ag1, (b) Ag6, (c) Ag8, (d) Ag10, and (e) Ag12 digested with trypsin, in which AgUox-WT was used as a control group;

FIG. 31 shows enzyme digestion electrophoresis results of pTAC-Uox-W174mb;

FIG. 32 shows a result of confirming the cloning sequence of pTAC-Uox-W174mb;

FIG. 33 shows analysis results of primary separation and purification for Uox-frTet through a DEAE column;

FIG. 34 shows SDS-PAGE analysis results of primary separation and purification for Uox-frTet through a DEAE column;

FIG. 35 shows analysis results of secondary separation and purification for Uox-frTet through a phenyl fast flow column;

FIG. 36 shows SDS-PAGE analysis results of secondary separation and purification for Uox-frTet through a phenyl fast flow column; and

FIG. 37 shows SDS-PAGE analysis results of Uox-frTet and Fasturtec.

### BEST MODE

### Definition of Terms

### About

As used herein, the term “about” refers to a degree close to a certain quantity, and it refers to an amount, level, value, number, frequency, percent, dimension, size, amount, weight, or length that varies by to the extent of 30%, 25%, 20%, 25%, 10%, 9%, 8%, 7%, 6%, 5%, 4%, 3%, 2%, or 1% with respect to a reference amount, level, value, number, frequency, percentage, dimension, size, amount, weight, or length.

### Click Chemistry

Herein, “Click Chemistry” is a term that was introduced by K. B. Sharpless in Scripps Research Institute to describe complementary chemical functional groups and chemical reactions designed such that two molecules can form a covalent bond fast and stably. The click chemistry does not mean a specific reaction but is a term for a fast and stable reaction. Click chemistry creates only byproducts that are not significant and is modular, wide in scope, high-yielding, stereospecific, biologically stable, large in thermodynamic dynamic (for example, 84 kJ/mol or more), and high in atomic economy. Example of the click chemistry include 1) Huisgen 1,3-dipolar cycloaddition (see Tornoe et al. Journal of Organic Chemistry (2002) 67: 3075-3064, etc.), 2) Diels-Alder reaction, 3) Nucleophilic addition to small strained rings such as epoxide and aziridine, 4) a nucleophilic addition reaction to an activated carbonyl group, and 5) an addition reaction to a carbon-carbon double bond or triple bond. The meaning of the click chemistry should be appropriately interpreted according to the context, and the click chemistry includes all other meanings that can be recognized by those skilled in the art.

### Bioorthogonal Reaction

Herein, the term “bioorthogonal reaction” refers to any chemical reaction in which externally introduced residues react with each other without interfering with native biochemical processes. When a certain reaction is “bioorthogonal”, the reaction has a characteristic that it is very stable in the body because in vivo intrinsic molecules are not involved in the reaction or reaction product.

### Standard Amino Acid

As used herein, the term “standard amino acid” refers to 20 amino acids synthesized through the transcription and translation processes of genes in the body of an organism. Specifically, the standard amino acid includes alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys K), methionine (Met, M), phenylalanine (Phe, F), proline (Pro, P), serine (Ser, S), threonine (Thr, T), tryptophan (Trp, W), tyrosine (Tyr, Y), and valine (Val, V). The standard amino acid has a corresponding DNA codon and can be represented by a general one-letter or three-letter notation of an amino acid. The subjects being referred to by the term standard amino acid should be appropriately interpreted according to the context, and they include all other meanings that can be recognized by those skilled in the art.

### Nonnatural Amino Acid

As used herein, the term “nonnatural amino acid” refers to an amino acid that is not synthesized in the body but synthesized artificially. The nonnatural amino acid includes, for example, 4-(1,2,3,4-tetrazin-3-yl) phenylalanine, and 4-(6-methyl-s-tetrazin-3-yl)phenylalanine. Since the nonnatural amino acid does not have a corresponding DNA codon, it cannot be represented by a general one-letter or three-letter notation of an amino acid, and it is written using other characters and explained via additional explanation. The subjects being referred to by the term nonnatural amino acids should be appropriately interpreted according to the context, and they include all other meanings that can be recognized by those skilled in the art.

### Description of Peptide Sequence

Unless otherwise stated, when describing the sequence of a peptide in the present specification, single letter notation or three letter notation of an amino acid is used, and it is written in the direction from the N-terminus to the C-terminus. For example, when expressed as RNVP, it refers to a peptide in which arginine, asparagine, valine, and proline are sequentially linked in the direction from the N-terminus to the C-terminus. For another example, when expressed as Thr-Leu-Lys, it refers to a peptide in which threonine, leucine, and lysine are sequentially linked in the direction from the N-terminus to the C-terminus. In the case of amino acids that cannot be represented by the one-letter or three-letter notation, other letters are used to describe these amino acids, and will be explained via additional explanation.

### Immunogenicity

As used herein, the term “immunogenicity” collectively refers to “the property of acting as an antigen capable of inducing an immune response” in the dictionary. There are various methods for measuring the immunogenicity of a specific antigen, and the methods may be appropriately adopted or designed according to the purpose. For example, the methods may include 1) a method for confirming whether IgG, IgA, and/or IgE type antibodies are generated in the body of a subject when the antigen is administered into the body of the subject, 2) a method for confirming the time when the IgG, IgA, and/or IgE type antibodies are generated depending on the administration cycle, 3) a method for confirming the titer of the induced antibodies to the antigen, and 4) when the mechanism of action of the induced antibodies is found, a method for measuring the effect according to the mechanism of action, but the methods are not limited thereto. The subjects being referred to by the term immunogenicity should be appropriately interpreted according to the context, and they include all other meanings that can be recognized by those skilled in the art.

### Albumination

### Therapeutic Protein

Therapeutic protein has been reported to be effective in the treatment of various diseases, and it is one of the important growth motives in the pharmaceutical industry. However, there is a problem in that therapeutic protein is continuously removed by glomerular filtration, pinocytosis, and immune response in a patient's body. Therefore, when developing a therapeutic protein, it is very important to extend the duration of the drug effect by lowering the rate at which it is removed from the patient's body due to such a phenomenon.

### Albumination

Human serum albumin has a long half-life of 2 weeks or longer in serum. This is because 1) due to the electrostatic repulsion of albumin molecules, filtration in the glomerulus does not easily occur, and 2) due to the recirculating action mediated by the neonatal Fc receptor (FcRn) in the endothelium, a period of in vivo degradation is long. When such albumin is chemically or physically bound to a drug molecule (for example, a therapeutic protein or compound and the like), the half-life of the drug molecule may be increased, where such technology is called albumination.

### Limitations in Related Art

### Limitations in PEGylation Technology

As existing technology for increasing in vivo stability, half-life, and the like of a therapeutic protein, there is a PEGylation technique in which polyethylene glycol is bound to the therapeutic protein. For example, KRYSTEXXA (pegloticase), a drug based on urate oxidase currently available in the market, is a drug whose half-life and the like are improved by PEGylation performed on urate oxidase. However, such PEGylation has limitations in that 1) polyethylene glycol is randomly attached and thus blocks the active sites of the therapeutic protein, thereby reducing the efficacy thereof, and 2) the polyethylene glycol has side effects of causing an allergic reaction in the body.

### Limitations in Albumination Technology Using SPAAC Reaction

The inventors have disclosed a urate oxidase-albumin conjugate in the literature “KR 1637010 B1”. The urate oxidase-albumin conjugate disclosed in the literature replaces one or more amino acids of a urate oxidase with a nonnatural amino acid and conjugates the urate oxidase with albumin using a linker having a dibenzocyclooctyne (DBCO) reactive group. However, the urate oxidase-albumin conjugate disclosed in the literature has a problem that the yield is very low due to the slow speed of the strain-promoted cycloaddition (SPAAC), which is the binding reaction of AzF and DBCO at the junction. Therefore, in the urate oxidase-albumin conjugate disclosed in the literature, despite the fact that there are at least four sites for albumin conjugation in a urate oxidase, there is a limitation in that only urate oxidase-albumin conjugates in which one or two albumins are conjugated per one urate oxidase can be obtained due to the inefficiency of the SPAAC reaction. Due to the limitations described above, the urate oxidase-albumin conjugate disclosed in the literature has problems in that 1) the effect of albumin conjugation, including an increase in the half-life or reduction in immunogenicity, is limited, 2) unexpected reactions may be occurred in the body due to exposure of the residue of AzF to which albumin is not conjugated.

In conclusion, the albumination technology disclosed in the aforementioned document has limitation in that the aforementioned problems makes expansion and application to other multimeric proteins difficult.

### Multimeric Protein-Albumin Conjugate

### Overview of Multimeric Protein-Albumin Conjugate

In the present disclosure, disclosed is a multimeric protein-albumin conjugate. The multimeric protein-albumin conjugate is one in which a multimeric protein variant and albumin are linked through a linker. In the multimeric protein-albumin conjugate, a predetermined number of albumins are conjugated through a linker to a multimeric protein variant having a properly modified amino acid sequence to site specifically form a conjugate. The multimeric protein variant has a form in which at least one amino acid in the amino acid sequence of the corresponding wild-type multimeric protein is substituted with a nonnatural amino acid, where the linker and albumin are linked through residues of the nonnatural amino acid.

Hereinafter, components constituting the protein multimer-albumin conjugate and the junction structure thereof will be described in detail.

### Multimeric Protein-Albumin Conjugate Component 1—Multimeric Protein Variant

The multimeric protein-albumin conjugate disclosed herein contains a multimeric protein variant. The multimeric protein variant has a form in which at least one amino acid sequence of the corresponding wild-type multimeric protein is substituted with a nonnatural amino acid. Specifically, the multimeric protein variant is formed by oligomerization of a plurality of monomer subunits. The monomer subunit has a form in which at least one amino acid sequence of the corresponding wild-type monomer subunit is substituted with a nonnatural amino acid. The multimeric protein-albumin conjugate is characterized in that a linker and residues of at least one nonnatural amino acid contained in the monomer protein variant are linked by causing an inverse electron-demand Diels-Alder reaction (IEDDA reaction), and albumin may be site-specifically conjugated depending on the incorporation site of the nonnatural amino acid.

### Multimeric Protein-Albumin Conjugate Component 2—Albumin

The albumin refers to human serum albumin and/or a variant of human serum albumin, and serves as a drug carrier for the multimeric protein variant. The albumin allows the multimeric protein-albumin conjugate to exhibit an improved in vivo half-life and low immunogenicity, compared to the case where the multimeric protein variant is present alone.

### Multimeric Protein-Albumin Conjugate Component 3—Linker

The linker, used for linking the multimeric protein variant and the albumin, has an IEDDA reactive group capable of binding to the multimeric protein variant, a thiol reactive moiety capable of binding to albumin, and an anchor. In the multimeric protein-albumin conjugate, the IEDDA reactive group and the thiol reactive moiety are bound to the multimeric protein variant and the albumin, respectively, and thus are not intact in the original forms contained in the linker, but deformed into a multimeric protein-linker junction and an albumin-linker junction, respectively.

### Multimeric Protein-Albumin Conjugate Structure 1—Subunit-Albumin Conjugate

The multimeric protein variant contained in the multimeric protein-albumin conjugate disclosed herein is formed by oligomerization of two or more monomer subunits, and is characterized in that a certain number or more of the monomer subunits are conjugated to albumin. Among the monomer subunits constituting the multimeric protein variant, one conjugated to albumin through the linker is referred to as a subunit-albumin conjugate.

The multimeric protein-albumin conjugate is characterized by being a multimer formed by oligomerization of a plurality of monomer subunits containing at least one subunit-albumin conjugate.

### Multimeric Protein-Albumin Conjugate Structure 2—Multimeric Protein-Linker Junction

The multimeric protein-albumin conjugate disclosed herein is one in which the multimeric protein variant and albumin are conjugated through the linker. In this case, a portion where the multimeric protein variant and the linker are conjugated is called a multimeric protein-linker junction. The multimeric protein variant and the linker are characterized by being conjugated through an IEDDA reaction.

### Multimeric Protein-Albumin Conjugate Structure 3—Albumin-Linker Junction

The multimeric protein-albumin conjugate disclosed herein is one in which the multimeric protein variant and albumin are conjugated through the linker. In this case, a portion where the albumin and the linker are conjugated is called an albumin-linker junction.

### Multimeric Protein-Albumin Conjugate Structure 4—Anchor

The linker has an IEDDA reactive group capable of being conjugated to the multimeric protein variant, a thiol reactive moiety capable of being conjugated to albumin, and an anchor linking the two in one. The anchor is a portion not involved in the reaction linking the multimeric protein and albumin, and thus is characterized in that the structure included in the linker remains intact in the multimeric protein-albumin conjugate.

### Example of Multimeric Protein-Albumin Conjugate Structure 1—View of Multimeric Protein

In one embodiment, the multimeric protein-albumin conjugate is represented by formula 1:

MP-[J₁-A-J₂-HSA]ₙ, [Formula 1]
where the MP is a multimeric protein variant,
the J₁ is a multimeric protein-linker junction,
the A is an anchor,
the J₂ is an albumin-linker junction, and
the HSA is human serum albumin,
in which the multimeric protein variant is a multimer formed by oligomerization of m monomer subunits,
the m is an integer of 2 or more and 6 or less, and
the n is an integer of 2 or more.

### Example of Multimeric Protein-Albumin Conjugate Structure 2—View of Multimeric Protein Subunit

In one embodiment, the subunit-albumin conjugate is represented by formula 2:

p′-J₁-A-J₂-HAS, [Formula 2]
where the p′ is a multimeric protein variant subunit, and the others are the same as defined above.

When the multimeric protein is composed of m subunits, the multimeric protein-albumin conjugate may be one in which n subunit-albumin conjugates and (m-n) monomer subunits are oligomerized to form a complex. In this case, substitution sites of nonnatural amino acids in the amino acid sequence of each of the monomer subunits and the subunit-albumin conjugates may be the same or different, where the n is an integer of m or less, which may vary depending on the m value.

### Multimeric Protein-Albumin Conjugate Characteristic 1—Applicable to Multimeric Protein Containing Plurality of Subunits

The inventors of the present disclosure have proved through experiments that a tetramer protein-albumin conjugate in which albumin is site-specifically conjugated to variants having partially modified amino acid sequences of representative tetramer proteins, which are urate oxidase, asparaginase, and methioninase, has an activity equivalent to or superior to that of each of the tetramer proteins found in nature and has the aforementioned advantages of albumination, such as improvement in in vivo half-life, reduction in immunogenicity, and the like. Furthermore, based on the experiments mentioned above, the inventors of the present disclosure have also discovered the site-specific albumination effect of protein multimers (for example, dimer to hexamer proteins) containing a plurality of subunits other than tetramers to complete the present disclosure.

The multimeric protein-albumin conjugate disclosed herein is characterized by being universally applicable to various multimeric proteins without being limited to a multimeric protein containing a predetermined number of subunits.

### Multimeric Protein-Albumin Conjugate Characteristic 2—Effect of Increasing Half-Life Through Albumin Conjugation

As described above, when conjugating albumin to drug molecules, there is an effect of increasing in vivo half-life. The multimeric protein-albumin conjugate provided herein is characterized by increasing in vivo half-life through the conjugation of albumin to the multimeric protein. The improved in vivo half-life may be confirmed through a pharmacokinetic profile experiment performed after administering the multimeric protein-albumin conjugate to the body.

### Multimeric Protein-Albumin Conjugate Characteristic 3—not Inhibiting Activity of Multimeric Protein

One of the limitations in the related art is that although a drug carrier, for example, albumin, polyethylene glycol (PEG), or the like, is bound to the multimeric protein to increase the efficacy thereof, such a drug carrier inhibits the activity by blocking the active sites of the multimeric protein due to the three-dimensional structure thereof, resulting in a reduction in drug efficacy. The multimeric protein-albumin conjugate disclosed herein is characterized in that the drug efficacy is not reduced by site-specifically conjugating albumin to a site that does not inhibit the activity of the multimeric protein.

### Multimeric Protein-Albumin Conjugate Characteristic 4—Effect of Reducing Immunogenicity

In many cases, multimeric proteins available as therapeutic agents are not human-derived proteins but are foreign-derived proteins such as microorganisms and the like. For example, when the multimeric protein is urate oxidase, it is known that humans do not produce the same, so microorganism-derived enzymes are used for treatment. In this case, the multimeric protein is a foreign protein and thus causes an immune response when administered alone to the body, resulting in side effects. Therefore, reducing the immunogenicity of these multimeric proteins is an important task. The multimeric protein-albumin conjugate disclosed herein is characterized by reducing the immunogenicity of the multimeric protein through the conjugation of the multimeric protein to albumin, which is a human plasma protein. The albumin, a substance constituting most of the plasma proteins, is extremely stable in the human body and hardly exhibits immunogenicity. Therefore, compared to the case where the multimeric protein is administered alone to the body, the multimeric protein-albumin conjugate exhibits significantly low immunogenicity.

### Multimeric Protein Variant

### Overview of Multimeric Protein Variant

A multimeric protein-albumin conjugate disclosed herein contains a multimeric protein variant. In this case, the multimeric protein variant has a form in which at least one amino acid sequence of the corresponding wild-type multimeric protein is substituted with a nonnatural amino acid for the purpose of site-specifically conjugating albumin.

### Monomer Subunit of Multimeric Protein Variant

The multimeric protein variant is characterized by being formed by oligomerization of a plurality of monomer subunits. For example, when the multimeric protein is a tetramer, the multimeric protein variant is formed by oligomerization of four monomer subunits. In this case, the monomer subunit may have a form in which at least one amino acid sequence of a wild-type monomer subunit is substituted with a nonnatural amino acid.

In one embodiment, the multimeric protein variant may be formed by oligomerization of 2 to 6 monomer subunits.

The monomer subunits will be described in detail in the section titled “Monomer subunit of multimeric protein variant”.

### Example of Multimeric Protein Variant

In one embodiment, the multimeric protein variant may be a multimeric protein variant selected from among the following:
urate oxidase; asparaginase; methioninase; elosulfase alfa; and glucarpidase.

In one embodiment, the multimeric protein variant may not show activity when being present in a monomer form, but show activity only when being present in a multimer form. Specifically, the multimeric protein variant may have a form in which an active site is present between the monomer subunits of each multimeric protein.

### Monomer Subunit of Multimeric Protein Variant

Substitution with Nonnatural Amino Acid

Compared to a wild-type monomer subunit, a monomer subunit of the multimeric protein variant is characterized in that at least one amino acid in the corresponding amino acid sequence is substituted with a nonnatural amino acid. The purpose is to site-specifically link the linker to residues of the nonnatural amino acid through an inverse electron-demand Diels-Alder reaction (IEDDA reaction), so 1) which amino acid at a certain site is to be substituted and 2) which nonnatural amino acid is used for substitution are significantly important.

### Substitution Site of the Non-Natural Amino Acid

When a Multimeric protein variant is formed by inserting a nonnatural amino acid into a wild Multimeric protein, the structure and function of the original Multimeric protein should not be affected as much as possible. Therefore, an amino acid that plays an important role in the activity and structure of a Multimeric protein cannot be substituted with a nonnatural amino acid. In addition, since the nonnatural amino acid needs to bind to the linker during the preparation of the Multimeric protein-albumin conjugate, it is advantageous to substitute the amino acid at a position with relatively high accessibility to a solvent, in the three-dimensional structure of the Multimeric protein. Various methods can be used to select sites with high solvent accessibility while minimally affecting the structure and function of the wild Multimeric protein. For example, molecular modeling calculations can select candidate sites that are similar in intrinsic atomic energy to the wild Multimeric protein and which are high in solvent accessibility.

In one embodiment, the site for substitution with a nonnatural amino acid in the sequence of the wild Multimeric protein to make a Multimeric protein variant may be determined by referring to molecular modeling simulation results. Specifically, the molecular modeling simulation result may be a scoring result of the Rosetta molecular modeling package.

### Example of Non-Natural Amino Acid 1—by Name

The multimer peptide variant subunit includes at least one nonnatural amino acid, and the nonnatural amino acid has a functional group capable of being bound to a linker through an IEDDA reaction. In one embodiment, the nonnatural amino acid may be an amino acid including a dien functional group capable of causing an IEDDA reaction. Specifically, the dien functional group may be a tetrazine functional group or a derivative thereof and/or a triazine functional group or a derivative thereof. More specifically, the nonnatural amino acid may be selected from the group consisting of 4-(1,2,3,4-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Example of Non-Natural Amino Acid 2—by Formula

In one embodiment, the nonnatural amino acid may be selected from the following:

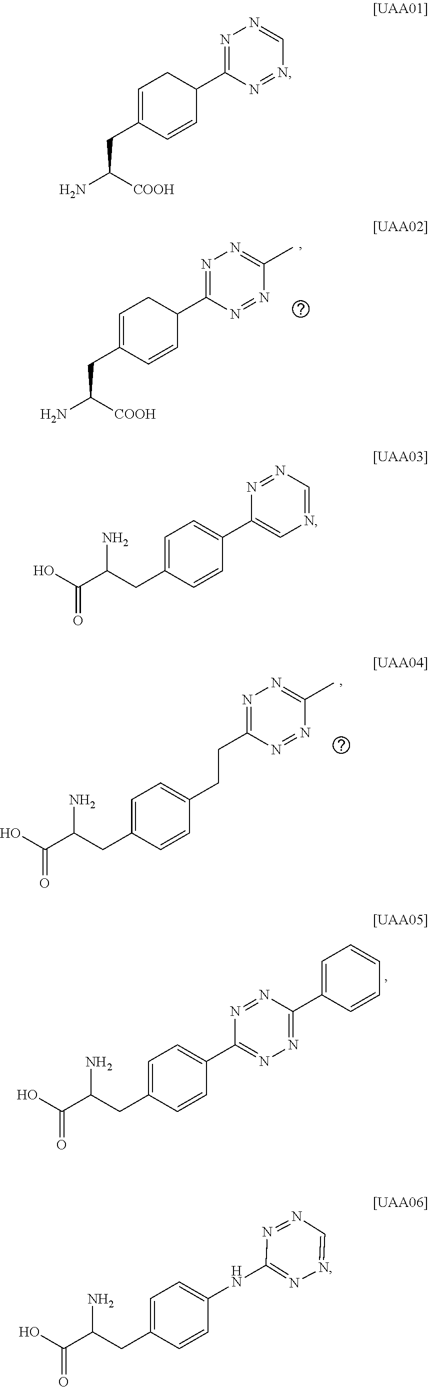 [SMILES: N[C@@H](CC1=CCC(c2nncnn2)C=C1)C(=O)O ; Cc1nnc(C2C=CC(C[C@H](N)C(=O)O)=CC2)nn1 ; NC(Cc1ccc(-c2cncnn2)cc1)C(=O)O ; Cc1nnc(CCc2ccc(CC(N)C(=O)O)cc2)nn1 ; NC(Cc1ccc(-c2nnc(-c3ccccc3)nn2)cc1)C(=O)O ; NC(Cc1ccc(Nc2nncnn2)cc1)C(=O)O]

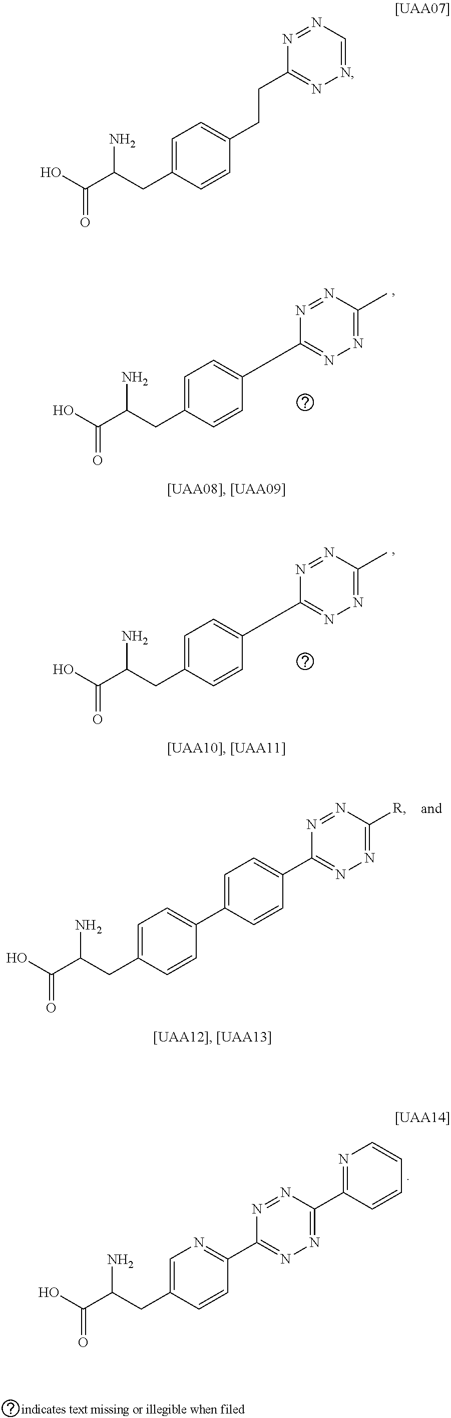

### Example of Multimeric Protein Variant 1—Urate Oxidase

### Overview of Urate Oxidase

Urate oxidase (Uricase) is a type of enzyme that cannot be synthesized in primates including humans, and it functions to break down uric acid into allantoin. The allantoin has a solubility 5 to 10 times higher than that of uric acid, so it is easy to be excreted by the kidneys. Therefore, when urate oxidase is used as a therapeutic agent, it is possible to treat gout by preventing the accumulation of uric acid, which is the main cause of gout, and by excreting uric acid from the body.

In one embodiment, the multimeric protein may be urate oxidase derived from Arthrobacter globiformis.

### Wild-Type Urate Oxidase Sequence

Wild-type urate oxidase is a tetramer protein in which four identical wild-type urate oxidase subunits are oligomerized.

In one embodiment, the wild-type urate oxidase is urate oxidase derived from Arthrobacter globiformis, and a peptide sequence of the subunit thereof may be MTATAETSTGTKVVLGQNQYGKAEVRLVKVTRNTARHEIQDLNVTSQLRGDFEAAHTAGDNAHVVATD TQKNTVYAFARDGFATTEEFLLRLGKHFTEGFDWVTGGRWAAQQFFWDRINDHDHAFSRNKSEVRTAV LEISGSEQAIVAGIEGLTVLKSTGSEFHGFPRDKYTTLQETTDRILATDVSARWRYNTVEVDFDAVYA SVRGLLLKAFAETHSLALQQTMYEMGRAVIETHPEIDEIKMSLPNKHHFLVDLQPFGQDNPNEVFYAA DRPYGLIEATIQREGSRADHPIWSNIAGFC (SEQ ID NO: 1) in the direction from the N-terminus to the C-terminus.

### Substitution Site of Urate Oxidase

In one embodiment, the urate oxidase variant subunit may be one in which a nonnatural amino acid is substituted for at least one selected from among aspartic acid at position 80, phenylalanine at position 82, phenylalanine at position 100, aspartic acid at position 101, phenylalanine at position 114, asparagine at position 119, aspartic acid at position 120, serine at position 142, glutamic acid at position 143, glycine at position 175, valine at position 195, glutamic acid at position 196, histidine at position 218, and proline at position 238, of the peptide sequence of SEQ ID NO: 1.

### Example Sequence of Urate Oxidase Variant

In one embodiment, the multimeric protein variant is one in which the sequence of urate oxidase derived from Aspergillus flavus is partially modified, and the urate oxidase variant subunit may be represented by SEQ ID NOs: 4 to 17. In this case, X of the sequence is selected from the group consisting of 4-(1,2,4,5-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Having Sequence Similar to Example Sequence of Urate Oxidase Variant

In one embodiment, the urate oxidase variant subunit may have a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 9%, 82%, 83%, 84%, 85%, 86%, 87%, 88%, 89%, 90%, 91%, 92%, 93%, 94%, 95%, 96%, 97%, 98%, 99%, or 100% identical to a sequence selected from among SEQ ID NOs: 4 to 17. In one embodiment, the urate oxidase variant subunit may have a sequence that is identical to a sequence selected from among SEQ ID NOs: 4 to 17 within a numerical range selected from the immediately preceding sentence. For example, the urate oxidase variant subunit may have a sequence that is 80% to 100% identical to a sequence selected from among SEQ ID NOs: 4 to 17. In another example, the urate oxidase variant subunit may have a sequence that is 95% or more identical to a sequence selected from among SEQ ID NOs: 4 to 17.

### Example of Multimeric Protein Variant 2—Asparaginase

### Overview of Asparaginase

Asparaginase, a protein that acts in a tetramer form to which four monomer subunits are bound, is an enzyme that breaks down asparagine, an amino acid. The asparaginase may be used as a therapeutic agent for acute lymphoblastic leukemia and kill cancer cells by depleting asparagine in the blood and blocking protein synthesis.

In one embodiment, the multimeric protein variant may be L-asparaginase. Specifically, the L-asparaginase may be a protein derived from Erwinia chrysanthemi.

### Wild-Type Asparaginase Sequence

Wild-type asparaginase is a tetramer protein in which four identical wild-type asparaginase subunits are oligomerized.

In one embodiment, the wild-type asparaginase is asparaginase derived from Erwinia chrysanthemi, and the subunit peptide sequence thereof may be MADKLPNIVILATGGTIAGSAATGTQTTGYKAGALGVDTLINAVPEVKKLANVKGEQFSNMASENMTG DVVLKLSQRVNELLARDVDGVVITHGTDTVEESAYFLHLTVKSDKPVVFVAAMRPATAISADGPMNLL EAVRVAGDKQSRGRGVMVVLNDRIGSARYITKTNASTLDTFKANEEGYLGVIIGNRIYYQNRIDKLHT TRSVFDVRGLTSLPKVDILYGYQDDPEYLYDAAIQHGVKGIVYAGMGAGSVSVRGIAGMRKAMEKGVV VIRSTRTGNGIVPPDEELPGLVSDSLNPAHARILLMLALTRTSDPKVIQEYFHTY (SEQ ID NO: 2) in the direction from the N-terminus to the C-terminus.

### Substitution Site of Asparaginase

In one embodiment, the asparaginase variant subunit may be one in which a nonnatural amino acid is substituted for glutamic acid at position 80, alanine at position 83, aspartic acid at position 85, aspartic acid at position 86, aspartic acid at position 113, lysine at position 146, tyrosine at position 185, histidine at position 204, arginine at position 207, tyrosine at position 235, glutamine at position 240, glycine at position 242, serine at position 255, serine at position 257, arginine at position 259, glutamic acid at position 269, lysine at position 270, proline at position 287, aspartic acid at position 288, glutamic acid at position 290, serine at position 316, lysine at position 319, and/or glutamic acid at position 323, of the peptide sequence of SEQ ID NO: 2.

### Example Sequence of Asparaginase Variant

In one embodiment, the multimeric protein variant is one in which the sequence of asparaginase derived from Erwinia chrysanthemi is partially modified, and the asparaginase variant subunit may be represented by SEQ ID NOs: 18 to 40 and 138. In this case, X of the sequence is selected from the group consisting of 4-(1,2,4,5-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Having Sequence Similar to Example Sequence of Asparaginase Variant

In one embodiment, the asparaginase variant subunit may have a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 9%, 82%, 83%, 84%, 85%, 86%, 87%, 88%, 89%, 90%, 91%, 92%, 93%, 94%, 95%, 96%, 97%, 98%, 99%, or 100% identical to a sequence selected from among SEQ ID NOs: 18 to 40 and 138. In one embodiment, the asparaginase variant subunit may have a sequence that is identical to a sequence selected from among SEQ ID NOs: 18 to 40 and 138 within a numerical range selected from the immediately preceding sentence. For example, the asparaginase variant subunit may have a sequence that is 80% to 100% identical to a sequence selected from among SEQ ID NOs: 18 to 40 and 138. In another example, the asparaginase variant subunit may have a sequence that is 95% or more identical to a sequence selected from among SEQ ID NOs: 18 to 40 and 138.

### Example of Multimeric Protein Variant 3—Methioninase

### Overview of Methioninase

Methioninase, a protein that acts in a tetramer form to which four monomer subunits are bound, is an enzyme that breaks down methionine, an amino acid. Tumor cells exhibit methionine auxotrophy and are thus more sensitive to regulation of methionine than normal tissues. In addition, depletion of methionine enables control of tumor-cell DNA methylation, cell cycle progression, and biosynthesis of polyamines and antioxidants, so it is known that regulation of methionine may play an important role in cancer treatment. Therefore, methioninase, which functions to break down methionine, may be used as a therapeutic agent for cancer.

In one embodiment, the multimeric protein variant may be L-methioninase. Specifically, the L-methioninase may be a protein derived from Pseudomonas putida.

### Wild-Type Methioninase Sequence

Wild-type methioninase is a tetramer protein in which four identical wild-type methioninase subunits are oligomerized.

In one embodiment, the wild-type methioninase is methioninase derived from Pseudomonas putida, and the subunit peptide sequence thereof may be HGSNKLPGFATRAIHHGYDPQDHGGALVPPVYQTATFTFPTVEYGAACFAGEQAGHFYSRISNPTLNL LEARMASLEGGEAGLALASGMGAITSTLWTLLRPGDEVLLGNTLYGCTFAFLHHGIGEFGVKLRHVDM ADLQALEAAMTPATRVIYFESPANPNMHMADIAGVAKIARKHGATVVVDNTYCTPYLQRPLELGADLV VHSATKYLSGHGDITAGIVVGSQALVDRIRLQGLKDMTGAVLSPHDAALLMRGIKTLNLRMDRHCANA QVLAEFLARQPQVELIHYPGLASFPQYTLARQQMSQPGGMIAFELKGGIGAGRRFMNALQLFSRAVSL GDAESLAQHPASMTHSSYTPEERAHYGISEGLVRLSVGLEDIDDLLADVQQALKASA (SEQ ID NO: 3) in the direction from the N-terminus to the C-terminus.

### Substitution Site of Methioninase

In one embodiment, the methioninase variant subunit may be one in which a nonnatural amino acid is substituted for asparagine at position 4, leucine at position 6, histidine at position 16, glutamic acid at position 43, alanine at position 71, serine at position 75, glycine at position 78, proline at position 102, glycine at position 128, lysine at position 130, alanine at position 137, glutamine at position 140, alanine at position 144, proline at position 148, arginine at position 151, asparagine at position 162, lysine at position 173, arginine at position 176, lysine at position 177, leucine at position 229, aspartic acid at position 231, arginine at position 232, glutamine at position 236, proline at position 283, and/or threonine at position 300, of the peptide sequence of SEQ ID NO: 3.

### Example Sequence of Methioninase Variant

In one embodiment, the multimeric protein variant is one in which the sequence of methioninase derived from Pseudomonas putida is partially modified, and the methioninase variant subunit may be represented by SEQ ID NOs: 41 to 45 and 139 to 158.

### Having Sequence Similar to Example Sequence of Methioninase Variant

In one embodiment, the methioninase variant subunit may have a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 9%, 82%, 83%, 84%, 85%, 86%, 87%, 88%, 89%, 90%, 91%, 92%, 93%, 94%, 95%, 96%, 97%, 98%, 99%, or 100% identical to a sequence selected from among SEQ ID NOs: 41 to 45 and 139 to 158. In one embodiment, the methioninase variant subunit may have a sequence that is identical to a sequence selected from among SEQ ID NOs: 41 to 45 and 139 to 158 within a numerical range selected from the immediately preceding sentence. For example, the methioninase variant subunit may have a sequence that is 80% to 100% identical to a sequence selected from among SEQ ID NOs: 41 to 45 and 139 to 158. In another example, the methioninase variant subunit may have a sequence that is 95% or more identical to a sequence selected from among SEQ ID NOs: 41 to 45 and 139 to 158.

### Multimeric Protein Variant Preparation Method

### Overview of Multimeric Protein Variant Preparation Method

The present description discloses a method of preparing a Multimeric protein variant. The following matters are involved in the Multimeric protein preparation method: a cell line to express a Multimeric protein variant; an exogenous suppressor tRNA to recognize a specific stop codon; a foreign tRNA synthetase; and a vector encoding a Multimeric protein variant in which a nonnatural amino acid is encoded with the stop codon. Here, the exogenous suppressor tRNA and the exogenous tRNA synthetase are not the suppressor tRNA and tRNA synthetase specific to the expression cell line but a suppressor tRNA and tRNA synthetase derived from cells different from the expression cell line. Therefore, the exogenous suppressor tRNA is characterized in that it does not react with the tRNA synthetase unique to the expression cell line. The exogenous tRNA synthetase i) reacts only with the exogenous suppressor tRNA and ii) shows activity only in the nonnatural amino acid to be included in the Multimeric protein variant. As a result, when the exogenous tRNA synthetase is used, the nonnatural amino acid is specifically linked to the exogenous suppressor tRNA so that the nonnatural amino acid can be introduced into the peptide sequence.

The Multimeric protein variant preparation method is a method in which 1) in the cell line, 2) the exogenous suppressor tRNA and the exogenous tRNA synthetase are involved in 4) expressing the Multimeric protein variant, 3) based on a vector encoding the Multimeric protein variant. In the Multimeric protein variant preparation method, the order of each process is not particularly limited if the Multimeric protein variant can be expressed in the cell line, and additional processes may be included if necessary.

### Cell Line Expressing Multimeric Protein Variant

The Multimeric protein variant preparation method is characterized in that it is obtained by expressing a Multimeric protein variant in a cell line. The Multimeric protein variant expression cell line is not particularly limited if it can produce a Multimeric protein variant. However, when a release factor recognizing the stop codon in the cell line normally functions, the release factor competes with the exogenous tRNA, thereby reducing the yield. Therefore, it is preferable to use a cell line in which the release factor that recognizes the stop codon is inactivated.

In one embodiment, the cell line expressing the multimeric protein variant may be selected from the following:

Escherichia genus; Erwinia genus; Serratia genus; Providencia genus; Corynebacterium genus; Pseudomonas genus; Leptospira genus; Salmonella genus; Brevibacterium genus; Hyphomonas genus; chromobacterium genus; norcardia genus; fungi; and yeast.

In one embodiment, the cell line may be a cell line in which a release factor that recognizes a stop codon and terminates translation is inactivated. Specifically, the stop codon is any one selected from among an amber codon (5′-UAG-3′), an ocher codon (5′-UAA-3′), and an opal codon (5′-UGA-3′).

In one embodiment, the cell line expressing the Multimeric protein variant may be the cell line used in the method disclosed in the literature “KR 1637010 B1”. Specifically, the cell line may be E.Coli C321.ΔA.exp (Addgene, ID: 49018).

Exogenous Suppressor tRNA

The exogenous suppressor tRNA is a tRNA that recognizes a specific stop codon, and does not react with a tRNA synthetase unique to the expression cell line. The exogenous suppressor tRNA specifically reacts with the exogenous tRNA synthetase, and the exogenous tRNA synthetase functions to link a nonnatural amino acid to the exogenous suppressor tRNA. As a result, the exogenous suppressor tRNA can recognize the specific stop codon and introduce the nonnatural amino acid at the corresponding position.

Specifically, the suppressor tRNA may recognizes any one selected from among an amber codon (5′-UAG-3′), an ocher codon (5′-UAA-3′), and an opal codon (5′-UGA-3′). Preferably, the suppressor tRNA may recognize an amber codon. For example, the suppressor tRNA may be a suppressor tRNA (MjtRNATyrCUA) derived from Methanococcus jannaschii (Yang et.al, Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464).

Exogenous tRNA Synthetase

The exogenous tRNA synthetase selectively reacts with a specific nonnatural amino acid, and functions to link the specific nonnatural amino acid to the exogenous suppressor tRNA. The exogenous tRNA synthetase does not react with the a suppressor tRNA unique to the expression cell line and specifically reacts with only the exogenous suppressor tRNA. In one embodiment, the tRNA synthetase may have a function of linking a nonnatural amino acid including a tetrazine derivative and/or a triazine derivative to the exogenous suppressor tRNA. In one embodiment, the tRNA synthetase may be a tyrosyl-tRNA synthetase (MjTyrRS) derived from Methanococcus jannaschii (Yang et.al, Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464). Preferably, the tRNA synthetase may be a C11 variant of the MjTyrRS.

Orthogonal tRNA/Synthetase Pair

In the present description, 1) an exogenous suppressor tRNA that specifically reacts with only the exogenous tRNA synthetase, and 2) the exogenous tRNA synthetase are collectively called an orthogonal tRNA/synthetase pair. In the Multimeric protein variant preparation method disclosed herein, it is important to express the orthogonal tRNA/synthetase pair in the expression cell line. The method is not particularly limited if this objective can be achieved. In one embodiment, the Multimeric protein variant preparation method includes transforming the cell line with a vector capable of expressing the orthogonal tRNA/synthetase pair. Specifically, the vector capable of expressing the orthogonal tRNA/synthetase pair may be pDUle_C11 reported by Yang et.al. (Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464).

### Vector Encoding Multimeric Protein Variant

The Multimeric protein variant preparation method includes a process of introducing or transfecting a vector encoding a Multimeric protein variant into an expression cell line. A more specific description will be provided in the section titled “Vector Encoding Multimeric protein Variant”.

### Example of Multimeric Protein Variant Preparation Method

In one embodiment, a Multimeric protein variant preparation method includes the following:
preparing a cell line including a vector capable of expressing an orthogonal tRNA/synthetase pair, and a vector encoding a Multimeric protein variant,
in which the orthogonal tRNA/synthetase pair includes an exogenous suppressor tRNA and an exogenous tRNA synthetase,
the Multimeric protein variant is a variant in which three or more amino acids in a wild-type Multimeric protein sequence are substituted with nonnatural amino acids each including a tetrazine derivative or a triazine derivative, and
in the vector encoding the Multimeric protein variant, the codon corresponding to the nonnatural amino acid is an amber codon (5′-UAG-3′); and
culturing the cell line in a medium which contains a nonnatural amino acid including a tetrazine functional group and/or a triazine functional group,
in which the exogenous suppressor tRNA can recognize the amber codon (5′-UAG-3′),
the exogenous tRNA synthetase may link the nonnatural amino acid to the exogenous tRNA, and
accordingly, when the cell line expresses the vector encoding the Multimeric protein variant, the cell line expresses a peptide in which the nonnatural amino acid is linked to a position corresponding to the amber codon.

### Vector Encoding Multimeric Protein Variant

### Overview of Vector Encoding Multimeric Protein Variant

The present description discloses a vector encoding a urate oxidase variant. The vector encoding a urate oxidase variant is characterized in that the nonnatural amino acid in the sequence of the urate oxidase variant is encoded with a stop codon. In one embodiment, in the vector encoding a urate oxidase variant, a standard amino acid in the sequence of the urate oxidase variant is encoded with a codon corresponding to a standard amino acid found in nature, and a nonnatural amino acid may be encoded with a stop codon. For example, the stop codon is any one selected from among an amber codon (5′-UAG-3′), an ocher codon (5′-UAA-3′), and an opal codon (5′-UGA-3′). Alternatively, the stop codon may be selected from among 5′-TAG-3′, 5′-TAA-3′, and 5′-TGA-3′. In one embodiment, the vector encoding a urate oxidase variant may be codon-optimized for the expression cell line. For example, the vector encoding a urate oxidase variant may be an E. coli codon-optimized one.

### Sequence Example of Vector Encoding Multimeric Protein Variant

In one embodiment, when the multimeric protein variant is one in which the amino acid sequence of urate oxidase is partially modified, the vector encoding the multimeric protein variant may have nucleic acid sequences of SEQ ID NOs: 58 to 71.

In one embodiment, when the multimeric protein variant is one in which the amino acid sequence of asparaginase is partially modified, the vector encoding the multimeric protein variant may have nucleic acid sequences of SEQ ID NOs: 100 to 104.

In one embodiment, when the multimeric protein variant is one in which the amino acid sequence of methioninase is partially modified, the vector encoding the multimeric protein variant may have nucleic acid sequences of SEQ ID NOs: 117 to 121.

### Having Sequence Similar to Example Sequence of Vector Encoding Multimeric Protein Variant

In one embodiment, the vector encoding the multimeric protein variant may have a sequence that is 50%, 51%, 52%, 53%, 54%, 55%, 56%, 57%, 58%, 59%, 60%, 7%, 62%, 63%, 64%, 65%, 66%, 67%, 68%, 69%, 70%, 71%, 72%, 73%, 74%, 75%, 76%, 77%, 78%, 79%, 80%, 9%, 82%, 83%, 84%, 85%, 86%, 87%, 88%, 89%, 90%, 91%, 92%, 93%, 94%, 95%, 96%, 97%, 98%, 99%, or 100% identical to a sequence selected from among SEQ ID NOs: 58 to 71, 100 to 104, and 117 to 121. In one embodiment, the vector encoding the multimeric protein variant may have a sequence that is identical to a sequence selected from among SEQ ID NOs: 58 to 71, 100 to 104, and 117 to 121 within a numerical range selected from the immediately preceding sentence. For example, the vector encoding the multimeric protein variant may have a sequence that is 80% to 100% identical to a sequence selected from among SEQ ID NOs: 58 to 71, 100 to 104, and 117 to 121. In another example, the vector encoding the multimeric protein variant may have a sequence that is 95% or more identical to a sequence selected from among SEQ ID NOs: 58 to 71, 100 to 104, and 117 to 121.

### Albumin

### Overview of Albumin

Albumin included in the urate oxidase-albumin conjugate disclosed herein refers to a conventional albumin protein. The albumin serves to increase the half-life of a urate oxidase by conjugating with a urate oxidase and/or to decrease immunogenicity. The albumin is not limited if it can have the above-described functions, and may be a wild-type albumin found in nature or a genetically engineered albumin (albumin variant) from a wild-type albumin.

### Example of Albumin

In one embodiment, the albumin may be mammalian albumin. Specifically, the albumin may be human serum albumin. In one embodiment, the albumin may be wild-type human serum albumin. In one embodiment, the albumin may be recombinant albumin genetically engineered from wild-type human serum albumin.

### Example of Sequence of Albumin

In one embodiment, the albumin may be represented by a sequence selected from SEQ ID NOs: 46 to 57.

### Including Sequence Similar to Exemplary Albumin Sequence

In one embodiment, the albumin may include a sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence selected from SEQ ID NOs: 133 to 144. In one embodiment, the albumin may include a sequence similar or identical to a sequence selected from SEQ ID NOs: 133 to 144 by a degree corresponding to one of the percentages described above. For example, the albumin may include a sequence similar or identical to a sequence selected from SEQ ID NOs: 133 to 144 by a degree in the range of 80% to 100%. Alternatively, the albumin may include a sequence similar or identical to a sequence selected from SEQ ID NOs: 133 to 144 by a degree in the range of 95% or more.

### Linker

### Overview of Linker

The urate oxidase-albumin conjugate disclosed herein is one in which a urate oxidase variant and albumin are conjugated through a linker. In this case, the linker refers to a material used to link a urate oxidase variant and an albumin when preparing the urate oxidase-albumin conjugate.

Specifically, the linker includes: an IEDDA reactive group; an anchor; and a thiol reactive group capable of binding to the albumin. In the process of preparing a urate oxidase-albumin conjugate, the urate oxidase variant and the linker bind to each other via an IEDDA reactive group, and the albumin and the linker bind to each other via the thiol reactive group. Specific bonding processes can be understood by referring to the relevant paragraph. Therefore, the linker of the urate oxidase-albumin conjugate does not exist in its original form, but exists in a form of 1) a urate oxidase-linker junction, 2) an anchor, and 3) an albumin-linker junction.

### Linker Structure 1—IEDDA Reactive Group

The linker includes an IEDDA reactive group capable of causing an inverse electron-demand Diels-Alder reaction (IEDDA reaction). The IEDDA reactive group is configured to be linked to the urate oxidase variant, and reacts with residues of nonnatural amino acids of the urate oxidase variant to form a urate oxidase-linker junction. In one embodiment, the IEDDA reactive group may include a dienophile functional group. Specifically, the IEDDA reactive group may be trans-cyclooctene or a derivative thereof.

In one embodiment, the IEDDA reactive group may be selected from the following:

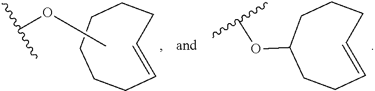

### Linker Structure 2—Thiol Reactive Group

The linker includes a thiol reactive group capable of reacting with thiol. The thiol group is configured to be linked to the albumin, and reacts with the thiol group included in the albumin to form an albumin-linker junction. In one embodiment, the thiol reactive group may be maleimide (MAL) or a derivative thereof, and/or 3-arylpropiolonitriles (APN) or a derivative thereof.

Specifically, the thiol reactive group may be selected from the following:

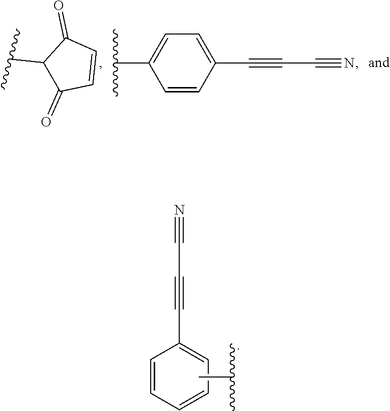

### Linker Structure 3—Anchor

The linker includes an anchor that links the IEDDA reactive group and the thiol reactive group. The anchor binds the IEDDA reactive group and the thiol reactive group into one molecule, and the structure of the anchor is not particularly limited as long as it does not affect the activity of the urate oxidase and/or albumin. In one embodiment, the anchor may have a linear structure. In another embodiment, the anchor may have a branched structure. In one embodiment, the anchor may include polyethylene glycol (PEG).

### Examples of Linker

In one embodiment, the linker may be any one selected from the following:

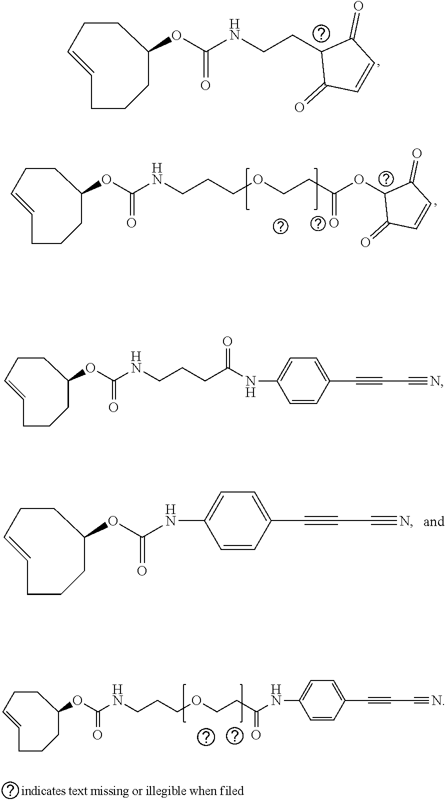

### Multimeric Protein-Linker Junction

### Overview of Multimeric Protein-Linker Junction

The Multimeric protein-albumin conjugate disclosed herein include a Multimeric protein-linker junction. The Multimeric protein-linker junction is generated by combining a Multimeric protein variant and a linker through an IEDDA reaction. Specifically, the IEDDA reaction refers to a reaction between the residue of the nonnatural amino acid of the Multimeric protein variant and the IEDDA reactive group of the linker, and, after the reaction, the structure of the Multimeric protein-linker junction is determined depending on the residue of the nonnatural amino acid and the type of the IEDDA reactive group. Since the Multimeric protein-albumin conjugate includes three or more albumin conjugates, the Multimeric protein-albumin conjugate includes three or more Multimeric protein-linker junctions. As described above, the Multimeric protein-albumin conjugate includes three or more subunit-albumin conjugates. The subunit-albumin conjugate is a structure in which a Multimeric protein variant subunit and an albumin are bound through a linker. Accordingly, each of the subunit-albumin conjugates includes at least one Multimeric protein-linker junction.

### Position of Multimeric Protein-Linker Junction

The Multimeric protein-linker junction is present at a position at which a nonnatural amino acid of a Multimeric protein variant and the anchor of a linker are linked. As described above, since the Multimeric protein-albumin conjugate is formed through the reaction of the residue of the nonnatural amino acid and the IEDDA reactive group, the Multimeric protein-albumin conjugate is positioned to correspond to the residue of the nonnatural amino acid of the Multimeric protein variant. In other words, the Multimeric protein-linker junction is present at a position corresponding to the IEDDA reactive group of the linker.

### Reaction for Forming Multimeric Protein-Linker Junction

The reaction for forming the Multimeric protein-linker junction is a kind of an inverse electron-demand Diels-Alder reaction (IEDDA reaction). A specific reaction mode may vary depending on the type of the functional group of the nonnatural amino acid of the Multimeric protein variant and the IEDDA reactive group of the linker. In one embodiment, the Multimeric protein-linker junction formation reaction may be any one of the following:

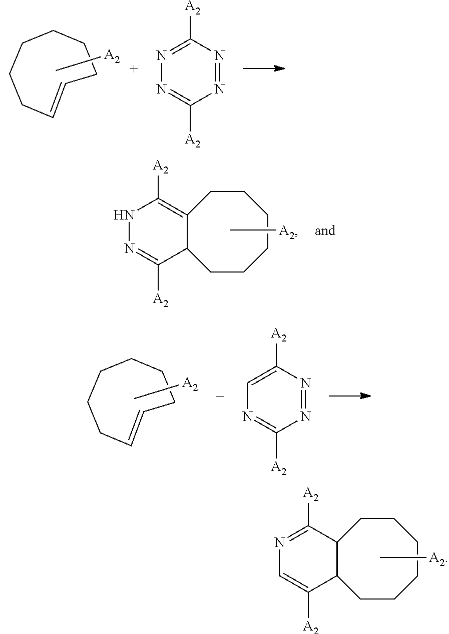

Here, A₂ is a linker portion excluding the IEDDA reactive group, Rx may vary depending on the type of the nonnatural amino acid (refer to the above-described examples of nonnatural amino acids), and A₁ is a Multimeric protein variant portion excluding a tetrazine functional group of a nonnatural amino acid.

### Structure of Multimeric Protein-Linker Junction

In one embodiment, structure of the Multimeric protein-linker junction may be any one of the following:

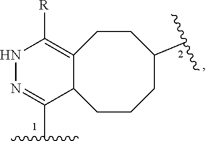

wherein, the R is selected from H, CH₃,

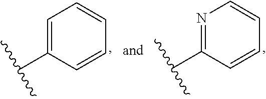 [SMILES: *c1ccccc1 ; *c1ccccn1]

the (1) part is linked to the Multimeric protein variant, and the (2) part is linked to the anchor of the linker; and

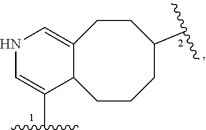

wherein, the (1) part is linked to the Multimeric protein variant, and the (2) part is linked to the anchor of the linker.

### Albumin-Linker Junction

### Overview of Albumin-Linker Junction

The Multimeric protein-albumin conjugate disclosed herein include a Multimeric protein-linker junction. The albumin-linker junction is generated by combining a thiol group included in albumin with a thiol group included in the linker. In this case, the thiol group of the albumin mediating the binding is characterized in that it is positioned to be spaced apart from the FcRn-binding domain of the albumin in order not to inhibit the half-life enhancing function of the albumin. Since the Multimeric protein-albumin conjugate includes three or more albumin conjugates, the Multimeric protein-albumin conjugate includes three or more albumin-linker junctions. As described above, the Multimeric protein-albumin conjugate includes three or more subunit-albumin conjugates. The subunit-albumin conjugate is a structure in which a Multimeric protein variant and an albumin are bound through a linker. Accordingly, each of the subunit-albumin conjugates includes at least one Multimeric protein-linker junction.

### Position of Albumin-Linker Junction

The albumin-linker junction is present at a position at which the thiol moiety of the albumin and the anchor of the linker are linked. Since the Multimeric protein-albumin conjugate disclosed herein has the purpose of increasing the half-life in the body by conjugating albumin to uric acid oxidase, the position where the albumin and the linker are connected must be a position spaced apart from the FcRn binding domain of albumin. As described above, since the Multimeric protein-albumin conjugate is formed by reacting the thiol group of the albumin and the thiol reactive group of the linker, the albumin-linker junction is present at a position corresponding to the thiol group of the albumin. In other words, the albumin-linker junction is present at a position corresponding to the thiol reactive group of the linker. The position of the albumin-linker junction is selected from among the thiol groups included in albumin, which do not affect the structure, function, and/or activity of albumin.

### Exemplary Position of Albumin-Linker Junction

In one embodiment, the albumin-linker junction may be located in a thiol group included in the residue of the 34th cysteine of albumin represented by SEQ ID NOs: 47 to 57.

### Albumin-Linker Junction Formation Reaction

The reaction for forming the albumin-linker junction is a kind of thiol reaction. A specific reaction mode may vary depending on the type of thiol group of the linker.

In one embodiment, the urate albumin-linker junction formation reaction may be any one of the following:

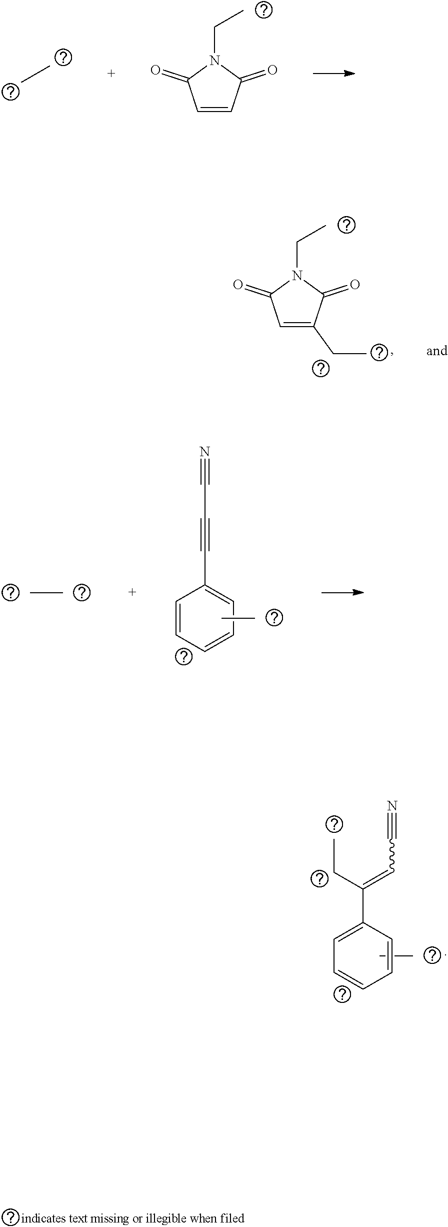

Here, R₁ is an albumin moiety excluding the thiol group, and R₂ is a linker moiety excluding the thiol group.

### Exemplary Structure of Albumin-Linker Junction

In one embodiment, the structure of the albumin-linker junction may be any one of the following:

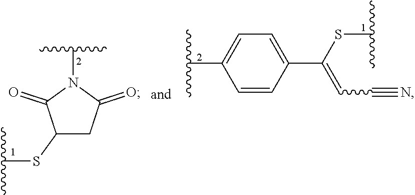 [SMILES: *SC1CC(=O)N(*)C1=O ; *SC(=CC#N)c1ccc(*)cc1]

here, the (1) part is linked to albumin, and the (2) part is linked to the anchor of the linker.

### Anchor

### Overview of Anchor

The anchor disclosed herein refers to a structure connected between the Multimeric protein-linker junction and the albumin-linker junction. The anchor binds the Multimeric protein variant, the Multimeric protein-linker junction, the albumin-linker junction, and the albumin into one structure. The anchor functions to regulate the distance between the Multimeric protein variant and the albumin in the Multimeric protein-albumin conjugate according to the structure thereof.

### Example of Structure of Anchor

In one embodiment, the anchor may be any one selected from the following:

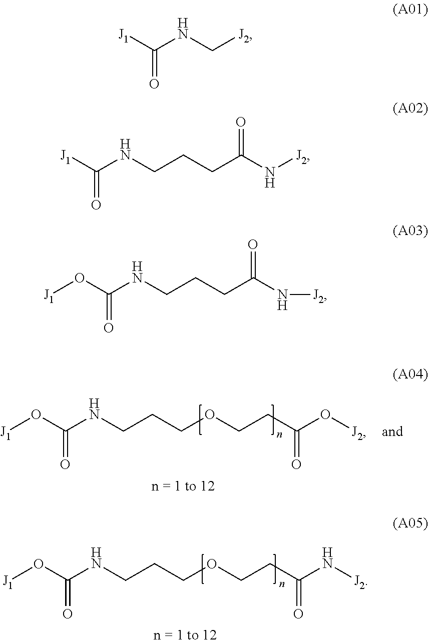

Herein J₁ is a Multimeric protein-linker junction, and J₂ is an albumin-linker junction.

### Multimeric Protein-Albumin Conjugate Preparation Method 1—General Methods

### Overview of Preparation Method for Multimeric Protein-Albumin Conjugate

The present description discloses a method of preparing a Multimeric protein-albumin conjugate. The following elements are involved in preparing a Multimeric protein-albumin conjugate: a Multimeric protein variant; a linker; and an albumin. Herein, the details of the elements are the same as described above.

The Multimeric protein-albumin conjugate preparation method is to prepare the above-described Multimeric protein-albumin conjugate by appropriately reacting each of the elements. Specifically, the Multimeric protein-albumin conjugate preparation method includes: reacting a nonnatural amino acid residue included in a Multimeric protein variant with an IEDDA reactive group of a linker to make a Multimeric protein-linker junction (Multimeric protein-linker conjugation reaction); and reacting a thiol group of an albumin with a thiol reactive group of a linker to form an albumin-linker junction (albumin-linker conjugation reaction). In this case, the order in which the uric acid oxidase-linker conjugation reaction and the albumin-linker conjugation reaction occur is irrelevant, and both reactions may occur simultaneously. In addition, depending on the sequence of each reaction, intermediate products of the reaction may be produced. The Multimeric protein-albumin conjugate preparation method will be described below in more detail.

### Multimeric Protein-Albumin Conjugate Preparation Method 1—Method of Binding Albumin and Linker First

In one embodiment, the Multimeric protein-albumin conjugate preparation method includes the following:
reacting an albumin and a linker,
in which a thiol moiety of the albumin and a thiol reactive moiety of the linker come into contact with each other to create an albumin-linker conjugate; and
reacting the albumin-linker conjugate and the Multimeric protein variant,
here, the IEDDA reactive group of the albumin-linker conjugate and the dien functional group of the nonnatural amino acid of the Multimeric protein variant come into contact to produce a Multimeric protein-albumin conjugate.

The Multimeric protein variant, the linker, and the albumin, and elements included therein are as described above.

### Multimeric Protein-Albumin Conjugate Preparation Method 2—Method of Binding Multimeric Protein and Linker First

In one embodiment, the Multimeric protein-albumin conjugate preparation method includes the following:
reacting a Multimeric protein variant and a linker,
here, the IEDDA reactive group of the linker and the dien functional group of the nonnatural amino acid of the Multimeric protein variant come into contact to produce a Multimeric protein-linker conjugate; and
reacting the Multimeric protein-linker conjugate and the albumin,
here, the thiol moiety of the albumin and the thiol moiety of the Multimeric protein-linker conjugate come into contact to produce a Multimeric protein-albumin conjugate.

The Multimeric protein variant, the linker, and the albumin, and elements included therein are as described above.

### Multimeric Protein-Albumin Conjugate Preparation Method 3—Method of Binding Multimeric Protein, Linker, and Albumin Simultaneously

In one embodiment, the Multimeric protein-albumin conjugate preparation method can be performed by adding all reactants and reacting them simultaneously.

In this case, the Multimeric protein-albumin conjugate preparation method includes the following:
reacting a Multimeric protein variant, a linker, and an albumin,
in which a thiol moiety of the albumin and a thiol moiety of the linker react to bind with each other,
the dien functional group contained in the nonnatural amino acid of the Multimeric protein variant and the IEDDA reactive group of the linker are conjugated through a reaction, and
as a result of the reaction, the Multimeric protein-albumin conjugate is produced.

The Multimeric protein variant, the linker, and the albumin, and elements included therein are as described above.

Characteristic of Multimeric protein-Albumin Conjugate Preparation Method 1—High Stability in Body due to bioorthogonal Reaction

In the method for preparing the Multimeric protein-albumin conjugate, the Multimeric protein variant and the albumin are conjugated through an IEDDA reaction, which is a kind of bioorthogonal reaction. Since a chemical functional group involved in the conjugation reaction does not exist in a molecule in the body, the Multimeric protein-albumin conjugate prepared by the preparation method has an advantage that the stability of the bond is very high even when introduced into the body.

### Characteristic of Multimeric Protein-Albumin Conjugate Preparation Method 2—High Yield Due to IEDDA Reaction

In the Multimeric protein-albumin conjugate preparation method, an inverse electron demand Diels-Alder reaction (IEDDA reaction) is used for conjugation of the Multimeric protein variant and the linker. Since the IEDDA reaction occurs at a very fast reaction rate and the reaction environment can be easily constructed, the yield is very high when preparing the conjugate compared to the case of using the Strain-Promoted Azide-Alkyne Cycloaddition (SPAAC) reaction.

### Multimeric Protein-Linker Conjugation Method

The Multimeric protein-albumin conjugate preparation method disclosed herein includes conjugating a Multimeric protein variant and a linker. Specifically, the Multimeric protein-linker conjugation method includes bringing the residue of the nonnatural amino acid of the Multimeric protein variant into contact with the IEDDA reactive group of the linker. The Multimeric protein-linker conjugation method is not affected by the site at which the albumin and the linker are conjugated or by whether the albumin and the linker are conjugated or not. Therefore, the Multimeric protein-linker conjugation method disclosed below is applicable to both the binding of the “linker” to the “Multimeric protein variant” and the binding of the “albumin-linker conjugate” to the “Multimeric protein variant”. The Multimeric protein-linker conjugation method may be performed independently of the albumin-linker conjugation method.

The Multimeric protein-linker conjugation method is not limited as long as it is a method capable of causing the reaction described in the section “Multimeric protein-Linker Junction Formation Reaction”, and a person skilled in the art may use a known method capable of causing the reaction.

Here, when the IEDDA reaction between the tetrazine functional group and the trans-cyclooctene functional group is caused by the Multimeric protein-linker conjugation method, the tetrazine functional group is reduced in a basic pH environment to increase the likelihood that the IEDDA reaction does not occur. Therefore, it is preferable that the IEDDA reaction proceeds in a neutral pH environment. In one embodiment, the Multimeric protein-linker conjugation method may be performed in a neutral pH environment. In one embodiment, the Multimeric protein-linker conjugation method may be performed in an environment of pH 8.0 or less, pH 9.0 or less, pH 10.0 or less, pH 11.0 or less, pH 11.0 or less, pH 13.0 or less, pH 14.0 or less.

### Albumin-Linker Conjugation Method

The Multimeric protein-albumin conjugate preparation method disclosed herein includes conjugating an albumin with a linker. Specifically, the albumin-linker conjugation method includes bringing the thiol moiety of the albumin into contact with the thiol moiety of the liner. The albumin-linker conjugation method is not affected by the site at which the Multimeric protein and the linker are conjugated or by whether the Multimeric protein and the linker are conjugated or not. Therefore, the albumin-linker conjugation method disclosed below is applicable to both the binding of the “linker” to the “albumin” and the binding of the “Multimeric protein-linker conjugate” to the “albumin”. The albumin-linker conjugation method may be performed independently of the Multimeric protein-linker conjugation method.

### Multimeric Protein-Albumin Conjugate Preparation Method 2—Novel Process

### Limitations in General Preparation Methods and Room for Improvement

Conventional processes for preparing multimeric protein-albumin conjugates involve a purification process whenever obtaining reactants from intermediate steps. For example, when preparing multimeric protein variants using a cell line as described in the section “Multimeric protein variant preparation method”, the following processes are required to be involved: 1) disrupting and purifying cells to obtain a multimeric protein variant, 2) purifying the resulting intermediate product after the reaction to obtain an albumin-linker conjugate or a multimeric protein-linker conjugate, and 3) purifying the resulting product at least three times to obtain a multimeric protein-albumin conjugate, which is an end product of the reaction. Typically, a purification process of a reaction product is not only time-consuming and costly but also affects the yield of the reaction product. Thus, in the case where the purification process can be omitted or simplified, a rather economical and effective process can be designed. However, in the case where the reactant or the reaction intermediate product of the reaction is not purified, the reaction proceeds in a state where the reactant and an impurity coexist. This may cause unintended reactions or generate unexpected reaction products, which makes it difficult to be controlled. Therefore, there is a limitation in that the purification process is unable to be omitted arbitrarily.

Based on the fact that the multimeric protein variant and the linker are linked through an inverse electron-demand Diels-Alder (IEDDA) reaction, one type of bioorthogonal reaction, the inventors of the present disclosure invented a method of efficienating the process by reducing the purification process.

### Overview of Novel Process for Preparing Multimeric Protein-Albumin Conjugate

In the present disclosure, disclosed is a novel process for preparing the multimeric protein-albumin conjugate. When preparing the multimeric protein variant using a cell line and adding the albumin-linker conjugate to a cell disruption product (lysed cell mixture), the resulting product of the cell line disruption, only the IEDDA reaction, which is the bioorthogonal reaction, selectively occurs rapidly. Since the multimeric protein-albumin conjugate, an end product, can be obtained after the reaction occurs, the purification process of the multimeric protein variant can be omitted. The novel process includes the following main processes: obtaining a cell disruption product by disrupting cells expressing the multimeric protein variant and adding the albumin-linker conjugate to the cell disruption product to cause the reaction.

### Cells Used in Novel Process

In the novel process provided herein, a cell containing the multimeric protein variant is used. The cell is a cell which expressed the multimeric protein variant, and the multimeric protein may be one described in the sections titled “Multimeric protein variant”, “Monomer subunit of multimeric protein variant”, “Example of multimeric protein variant 1—Urate oxidase”, “Example of multimeric protein variant 2—Asparaginase”, and “Example of multimeric protein variant 3—Methioninase”.

In one embodiment, the cell may be a cell line expressing the multimeric protein variant. Specifically, the cell may contain the exogenous suppressor tRNA, the exogenous tRNA synthetase, and/or the vector encoding the multimeric protein variant described in the section titled “Multimeric protein variant preparation method”.

### Cell Disruption Process

The novel process provided herein includes a cell disruption process. In this case, the cell disruption process is not particularly limited as long as the process makes the multimeric protein variant into a form capable of reacting with exogenous materials, and may be performed using known methods recognizable by those skilled in the art.

### Cell Disruption Product

A byproduct obtained as a result of cell disruption is called a cell disruption product. In this case, the cell disruption product refers to not only a product obtained immediately after performing the cell disruption process but also one involving appropriate and necessary treatment before adding the albumin-linker conjugate to cause a reaction.

In one embodiment, the cell disruption product may be the resulting product subjected to pretreatment performed on the byproduct obtained after performing the cell disruption process. Specifically, the pretreatment may be centrifugation, supernatant collection, filtering, chromatographic purification, and/or combinations thereof.

### Addition Process of Albumin-Linker Conjugate to Cell Disruption Product

The novel process provided herein includes an addition process of the albumin-linker conjugate to the cell disruption product. In this case, the “addition process” is not particularly limited as long as the process enables the albumin-linker conjugate and the multimeric protein variant to react.

### Albumin-Linker Conjugate

The albumin-linker conjugate is one in which the albumin described in the section titled “Albumin” and the linker described in the section titled “Linker” are linked through a thiol moiety of the albumin and a thiol reactive moiety of the linker, whose junction is as described in the section titled “Albumin-linker junction”.

In one embodiment, the albumin-linker conjugate is represented by Formula 3:

I-A-J-HAS, [Formula 3]
where I is an IEDDA reactive group, one of those described in the section titled “Linker structure 1—IEDDA reactive group”,
A is an anchor, one of those described in the section titled “Anchor”,
J is an albumin-linker junction, one of those described in the section titled “Albumin-linker junction”, and
HSA is albumin, one of those described in the section titled “Albumin”.

### Obtaining Process of Multimeric Protein-Albumin Conjugate

The novel process provided herein includes an obtaining process of the multimeric protein-albumin conjugate. The obtaining process is not particularly limited as long as the method enables the multimeric protein-albumin conjugate, which is a target end product, to be obtained in a usable form, and may be performed using known methods recognizable by those skilled in the art.

### Novel Process Characteristic 1—Shortening Production Process

The novel process is designed based on characteristics in which the inverse electron-demand Diels-Alder reaction (IEDDA reaction), one type of bioorthogonal reaction as well as click chemistry, hardly occurs between native molecules, but selectively occurs extremely rapidly between the IEDDA reactive groups. The novel process induces a reaction for producing a multimeric protein-albumin conjugate, an end product, by adding an albumin-linker conjugate directly to a cell disruption product and requires to undergo only a purification process to obtain the end product, and thus is characterized in that the production process is shortened compared to typical production methods.

### Novel Process Characteristic 2—Having High Yield Using IEDDA Reaction

The novel process also utilizes the IEDDA reaction and thus is characterized by having an extremely high yield of the end product due to rapid reaction speed, which results in a further high yield in the novel process, compared to that in typical production methods, by synergizing with the characteristic described above.

### Novel Process Characteristic 3—Suitable for Mass Production

The novel process includes a further simplified process, has a rather high yield of the end product, thereby being further economical and effective, and thus is characterized by being suitable for mass production.

### Use of Multimeric Protein-Albumin Conjugate

### Overview of Use of Multimeric Protein-Albumin Conjugate

In the present disclosure, disclosed is a use of a multimeric protein-albumin conjugate. The multimeric protein-albumin conjugate, obtained through albumination of a multimeric protein, has increased in vivo stability and exhibits effects of improving half-life, reducing immunogenicity, and the like. Therefore, when the multimeric protein has preventive or therapeutic use for a particular disease, disorder, and/or indication, the multimeric protein-albumin conjugate is available for the same use.

In one embodiment, the multimeric protein-albumin conjugate may be used for preventive or therapeutic use for a particular disease, disorder, and/or indication.

Preventive and/or Therapeutic Use of Multimeric Protein-Albumin Conjugate 1—Indication

In one embodiment, when the multimeric protein-albumin conjugate is a urate oxidase-albumin conjugate, it is available for preventive or therapeutic use for hyperuricemia, acute gouty arthritis, intermittent gout and chronic nodular gout, chronic kidney disease, and/or tumor lysis syndrome (TLS).

In one embodiment, when the multimeric protein-albumin conjugate is an asparaginase-albumin conjugate, it is available for therapeutic use for acute lymphoblastic leukemia.

In one embodiment, when the multimeric protein-albumin conjugate is a methioninase-albumin conjugate, it is available for therapeutic use for cancer.

In one embodiment, when the multimeric protein-albumin conjugate is an elosulfase alfa-albumin conjugate, it is available for therapeutic use for mucopolysaccharidosis IV A and/or Morquio A syndrome.

In one embodiment, when the multimeric protein-albumin conjugate is a glucarpidase-albumin conjugate, it is available for therapeutic use for anticancer drug toxicity and/or methotrexate poisoning.

Preventive and/or Therapeutic Use of Multimeric Protein-Albumin Conjugate 2—Administration Method

In one embodiment, the multimeric protein-albumin conjugate may be administered to patients through appropriate formulation to prevent or treat desired diseases, disorders, and/or indications. For example, the administration method may be one selected from oral administration, parenteral administration, intravenous administration, intraperitoneal administration, intramuscular administration, transdermal administration, and subcutaneous administration. Alternatively, the administration may be intravenous infusion.

Preventive and/or Therapeutic Use of Multimeric Protein-Albumin Conjugate 3—Dosage

In one embodiment, an appropriate dose of the multimeric protein-albumin conjugate may be administered to patients through appropriate formulation to prevent or treat desired diseases, disorders, and/or indications. For example, the dosage may be 0.01 mg/kg to 1000 mg/kg based on the Multimeric protein-albumin conjugate.

Preventive and/or Therapeutic Use of Multimeric Protein-Albumin Conjugate 4—Administration Interval

In one embodiment, the multimeric protein-albumin conjugate may be administered to patients through appropriate formulation to prevent or treat desired diseases, disorders, and/or indications at appropriate intervals. For example, the administration interval may be once a day. That is, an interval at which the appropriate dose of the Multimeric protein-albumin conjugate may be administered once a day. Alternatively, the Multimeric protein-albumin conjugate may be administered two times a day. In this case, the dosage per administration is half the appropriate dose per day. Further alternatively, the administration interval may be 1 hour, 2 hours, 6 hours, 12 hours, 24 hours, 2 days, 3 days, one week, two weeks, one month, or three months for the appropriate dosage of the Multimeric protein-albumin conjugate.

### Pharmaceutical Composition Including Multimeric Protein-Albumin Conjugate

### Overview of Pharmaceutical Composition Including Multimeric Protein-Albumin Conjugate

To use the Multimeric protein-albumin conjugate for desired diseases, disorders, and/or indications, the Multimeric protein-albumin conjugate must undergo appropriate formulation. Disclosed herein is a pharmaceutical composition suitably formulated to use a Multimeric protein-albumin conjugate as a therapeutic agent, and a pharmaceutically acceptable carrier required for formulation is disclosed. For example, the Multimeric protein-albumin conjugate may be formulated for oral use, parenteral use, injection, aerosol, and/or transdermal use, and may include a pharmaceutically acceptable carrier for this purpose.

### Composition for Formulation 1—Oral Preparations

In one embodiment, the Multimeric protein-albumin conjugate may be formulated as troches, lozenges, tablets, aqueous suspensions, oily suspensions, prepared powders, granules, emulsions, hard capsules, soft capsules, syrups, or elixirs.

In one embodiment, to formulating the Multimeric protein-albumin conjugate as oral preparations, the following may be used: binders such as lactose, saccharose, sorbitol, mannitol, starch, amylopectin, cellulose or gelatin; excipients such as dicalcium phosphate and the like; disintegrants such as corn starch or sweet potato starch; and lubricants such as magnesium stearate, calcium stearate, sodium stearyl fumarate or polyethylene glycol wax. In addition, sweeteners, air fresheners, and syrups may be used. Furthermore, in the case of capsules, in addition to the above-mentioned substances, a liquid carrier such as fatty oil may be additionally used.

### Composition 2 for Formulation—Parenteral Preparations

In one embodiment, the Multimeric protein-albumin conjugate may be formulated as an injection solution, suppository, powder for respiratory inhalation, aerosol for spray, ointment, powder for application, oil, or cream.

In one embodiment, in order to formulate the Multimeric protein-albumin conjugate for parenteral administration, a sterile aqueous solution, a non-aqueous solvent, a suspension, an emulsion, a freeze-dried preparation, an external preparation, etc. may be used. As the non-aqueous solvent and the suspension, propylene glycol, polyethylene glycol, vegetable oils such as olive oil, and injectable esters such as ethyl oleate may be used.

### Composition 3 for Formulation—Injection Preparations

In one embodiment, in order to formulate the uric acid oxidase-albumin conjugate as an injection solution, the binder for the Multimeric protein-albumin conjugate is mixed with a stabilizer or buffer in water to prepare a solution or suspension, and the solution or suspension may be formulated to be administered in units of an ampoule or vial.

### Composition 4 for Formulation—Aerosol Preparations

In one embodiment, the binder for the uric acid oxidase-albumin conjugate may be mixed with a propellant along with additives to prepare an aqueous dispersion concentrate or wet powder which may be subsequently formulated as aerosol preparations.

### Composition 5 for Formulation—Transdermal Preparations

In one embodiment, when the uric acid oxidase-albumin conjugate is formulated for transdermal use, animal oil, vegetable oil, wax, paraffin, starch, tragacanth, cellulose derivative, polyethylene glycol, silicone, bentonite, silica, talc, zinc oxide, etc. may be added as a carrier to the binder for the Multimeric protein-albumin conjugate to prepare ointment, cream, powder for application, oil, external preparation for skin, etc.

### Composition 6 for Formulation—Adjuvants and Other Components

In one embodiment, the pharmaceutical composition including the Multimeric protein-aluminum conjugate may include: water, saline, dextrose, ethanol, glycerol, sodium chloride, dextrose, mannitol, sorbitol, lactose, gelatin, albumin, aluminum hydroxide, Freund's incomplete adjuvant and complete adjuvant (Pifco Laboratories, Detroit, Mich.), Merck Adjuvant 65 (Merck and Company, Inc., Rahway, NJ.), Alhydrogel (Al(OH)3), aluminum hydroxide gel (alum), or aluminum salt such as aluminum phosphate, ASO4 series, MF, squalene, MF59, QS21, calcium, iron or zinc salt, insoluble suspension of acylated tyrosine, acylated fructose, cationically or anionically derived polysaccharides, polyphosphazenes, biodegradable microspheres, and Quil A, toll-like receptor (TLR) agonists, PHAD [Avanti polar lipid, Monophosphoryl Lipid A (synthetic)], monophosphoryl lipid A (MPL), synthetic lipid A, lipid A mimics or analogues, aluminum salts, cytokines, saponins, prolactin, growth hormone deoxycholic acid, betaglucan, polyribonucleotides, muramyl dipeptide (MDP) derivatives, CpG oligo, lipopolysaccharide (LPS) of Gram-negative bacteria, polyphosphazene, emulsion, virosome, cochleate, poly(lactide-co-glycolide) (PLG) microparticles, poloxamer particles, microparticles, liposomes, or suitable combinations thereof.

### Possible Embodiments

### Multimeric Protein-Albumin Conjugate 1

### Embodiment 1, Multimeric Protein-Albumin Conjugate

A multimeric protein-albumin conjugate represented by the following Structural Formula 1:

MP-[J₁-A-J₂-HSA]ₙ, [Structural Formula 1]
where the MP is a multimeric protein variant, the J₁ is a multimeric protein-linker junction, the A is an anchor, the J₂ is an albumin-linker junction, and the HAS is human serum albumin,
in which the multimeric protein variant contains m monomer subunits,
at least one of the m monomer subunits is a variant subunit, where the variant subunit contains at least one nonnatural amino acid having a diene functional group,
the multimeric protein variant contains n or more nonnatural amino acids,
the multimeric protein variant-linker junction has a junction structure formed through an inverse electron-demand Diels-Alder (IEDDA) reaction between the diene functional group of the nonnatural amino acid and dienophile functional group linked to the anchor,
the m is an integer of 2 or more, and the n is an integer of 1 or more and m or less.

### Embodiment 2, Dimer Protein-Albumin Conjugate

The protein-albumin conjugate of Embodiment 1, in which the m is 2, and the multimeric protein variant contains a subunit in combination selected from among the following:
1 wild-type subunit and 1 variant subunit; and
2 variant subunits.

### Embodiment 3, Trimer Protein-Albumin Conjugate

The protein-albumin conjugate of Embodiment 1, in which the m is 3, and the multimeric protein variant contains a subunit in combination selected from among the following:
2 wild-type subunits and 1 variant subunit;
1 wild-type subunit and 2 variant subunits; and
3 variant subunits.

### Embodiment 4, Tetramer Protein-Albumin Conjugate

The protein-albumin conjugate of Embodiment 1, in which the m is 4, and the multimeric protein variant contains a subunit in combination selected from among the following:
3 wild-type subunits and 1 variant subunit;
2 wild-type subunits and 2 variant subunits;
1 wild-type subunit and 3 variant subunits; and
4 variant subunits.

### Embodiment 5, Example of Tetramer Protein

The multimeric protein-albumin conjugate of Embodiment 4, in which the multimeric protein variant is characterized in that at least one amino acid sequence of the wild-type tetramer protein selected from among the following is substituted with the nonnatural amino acid:

urate oxidase; asparaginase; methioninase; and glucarpidase.

### Embodiment 6, Limitation of Tetramer Protein, Urate Oxidase

The multimeric protein-albumin conjugate of Embodiment 5, in which the multimeric protein variant is characterized in that at least one amino acid sequence of wild-type asparaginase derived from Erwinia chrysanthemi is substituted with the nonnatural amino acid.

### Embodiment 7, Limitation of Asparaginase Sequence

The multimeric protein-albumin conjugate of Embodiment 6, in which a sequence of the variant subunit contained in the multimeric protein variant is selected from among the following:

(SEQ ID NO: 18)
MADKLPNIVILATGGTIAGSAATGTQTTGYKAGALGVDTLINAVPEVKKLANVKGEQFSNMAS
ENMTGDVVLKLSQRVNXLLARDDVDGVVITHGTDTVEESAYFLHLTVKSDKPVVFVAAMRPATAISAD
GPMNLLEAVRVAGDKQSRGRGVMVVLNDRIGSARYITKTNASTLDTFKANEEGYLGVIIGNRIYYQNR
IDKLHTTRSVFDVRGLTSLPKVDILYGYQDDPEYLYDAAIQHGVKGIVYAGMGAGSVSVRGIAGMRKA
MEKGVVVIRSTRTGNGIVPPDEELPGLVSDSLNPAHARILLMLALTRTSDPKVIQEYFHTY;
(SEQ ID NO: 19)
MADKLPNIVILATGGTIAGSAATGTQTTGYKAGALGVDTLINAVPEVKKLANVKGEQFSNMASENMTG
DVVLKLSQRVNELLXRDDVDGVVITHGTDTVEESAYFLHLTVKSDKPVVEVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRTSDPKVIQEYFHTY;
(SEQ ID NO: 20)
MADKLPNIVILATGGTIAGSAATGTQTTGYKAGALGVDTLINAVPEVKKLANVKGEQFSNMASENMTG
DVVLKLSQRVNELLARXDVDGVVITHGTDTVEESAYFLHLTVKSDKPVVFVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 21)
MADKLPNIVILATGGTIAGSAATGTQTTGYKAGALGVDTLINAVPEVKKLANVKGEQFSNMASENMTG
DVVLKLSQRVNELLARDXVDGVVITHGTDTVEESAYFLHLTVKSDKPVVFVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 22)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSXKPVVFVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 23)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDXQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 24)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGXLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 25)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLX
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 26)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTFKANEEGYLGVIIGNRIYYQNRIDKLH
TTXSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 27)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLXDAAIQHGVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 28)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLYDAAIXHGVKGIVYAGMGAGSVSVRGIAGMRKAMEKGV
VVIRSTRTGNGIVPPDEELPGLVSDSLNPAHARILLMLALTRTSDPKVIQEYFHTY;
(SEQ ID NO: 29)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHXVKGIVYAGMGAGSVSVRGIAGMRKAMEKGV
VVIRSTRTGNGIVPPDEELPGLVSDSLNPAHARILLMLALTRISDPKVIQEYFHTY;
(SEQ ID NO: 30)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGXVSVRGIAGMRKAMEKGV
VVIRSTRTGNGIVPPDEELPGLVSDSLNPAHARILLMLALTRISDPKVIQEYFHTY;
(SEQ ID NO: 31)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVXVRGIAGMRKAMEKGV
VVIRSTRIGNGIVPPDEELPGLVSDSLNPAHARILLMLALTRISDPKVIQEYFHTY;
(SEQ ID NO: 32)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVXGIAGMRKAMEKGV
VVIRSTRTGNGIVPPDEELPGLVSDSLNPAHARILLMLALTRISDPKVIQEYFHTY;
(SEQ ID NO: 33)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLYDAAIQHGVKGIVYAGMGAGSVSVRGIAGMRKAMXKGV
VVIRSTRTGNGIVPPDEELPGLVSDSLNPAHARILLMLALTRISDPKVIQEYFHTY;
(SEQ ID NO: 34)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEXGV
VVIRSTRTGNGIVPPDEELPGLVSDSLNPAHARILLMLALTRISDPKVIQEYFHTY;
(SEQ ID NO: 35)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVFVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPXDEELPGLVSDSLNPAHARILLMLALTRTSDPKVIQEYFHTY;
(SEQ ID NO: 36)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPXEELPGLVSDSLNPAHARILLMLALTRISDPKVIQEYFHTY;
(SEQ ID NO: 37)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDXELPGLVSDSLNPAHARILLMLALTRISDPKVIQEYFHTY;
SEQ ID NO: 38)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRIXDPKVIQEYFHTY;
(SEQ ID NO: 39)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRTSDPXVIQEYFHTY;
(SEQ ID NO: 40)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTEKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEELPGLVSDSLNPAHARILLMLALTRISDPKVIQXYFHTY;
and
(SEQ ID NO: 138)
MADKLPNIVILATGGTIAGSAATGTQTTGYKAGALGVDTLINAVPEVKKLANVKGEQFSNMASENMTG
DVVLKLSQRVNELLARDDVDGVVITHGTDTVEESAYFLHLTVKSDKPVVEVAAMRPATAISADGPMNL
LEAVRVAGDKQSRGRGVMVVLNDRIGSARYITKTNASTLDTFKANEEGYLGVIIGNRIYYQNRIDKLH
TTRSVFDVRGLTSLPKVDILYGYQDDPEYLYDAAIQHGVKGIVYAGMGAGSVSVRGIAGMRKAMEKGV
VVIRSTRTGNGIVPPDEXLPGLVSDSLNPAHARILLMLALTRISDPKVIQEYFHTY,

where the X is one of the nonnatural amino acids exemplified in the section titled “Example of nonnatural amino acid 2—by formula”.

### Embodiment 8, Limitation of Tetramer Protein, Methioninase

The multimeric protein-albumin conjugate of Embodiment 5, in which the multimeric protein variant is characterized in that at least one amino acid sequence of wild-type methioninase derived from Pseudomonas putida is substituted with the nonnatural amino acid.

### Embodiment 9, Limitation of Methioninase Sequence

The multimeric protein-albumin conjugate of Embodiment 8, in which a sequence of the variant subunit contained in the multimeric protein variant is selected from among the following:

(SEQ ID NO: 41)
HGSNKLPGFATRAIHHGYDPQDHGGALVPPVYQTATFTFPTVEYGAACFAGEQAGHFYSRISN
PTLNLLEARMASLEGGEAGLALASGMGAITSTLWILLRPGDEVLLGNTLYGCTFAFLHHGIGEFGVKL
RHVDMADLQALEXAMTPATRVIYFESPANPNMHMADIAGVAKIARKHGATVVVDNTYCTPYLQRPLEL
GADLVVHSATKYLSGHGDITAGIVVGSQALVDRIRLQGLKDMTGAVLSPHDAALLMRGIKTLNLRMDR
HCANAQVLAEFLARQPQVELIHYPGLASFPQYTLARQQMSQPGGMIAFELKGGIGAGRRFMNALQLFS
RAVSLGDAESLAQHPASMTHSSYTPEERAHYGISEGLVRLSVGLEDIDDLLADVQQALKASA;
(SEQ ID NO: 42)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTXATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 43)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARXHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 44)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQXQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 45)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYXLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 139)
HGSXKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 140)
HGSNKXPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 141)
HGSNKLPGFATRAIHXGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 142)
HGSNKLPGFATRAIHHGYDPQDHGGALVPPVYQTATFTFPTVXYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 143)
HGSNKLPGFATRAIHHGYDPQDHGGALVPPVYQTATFTFPTVEYGAACFAGEQAGHFYSRISNPTLNL
LEXRMASLEGGEAGLALASGMGAITSTLWILLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 144)
HGSNKLPGFATRAIHHGYDPQDHGGALVPPVYQTATFTFPTVEYGAACFAGEQAGHFYSRISNPTLNL
LEARMAXLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 145)
HGSNKLPGFATRAIHHGYDPQDHGGALVPPVYQTATFTFPTVEYGAACFAGEQAGHFYSRISNPTLNL
LEARMASLEXGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 146)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRXGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRREMNALQLFSRAVSL
GDAESLAQHPASMTHSSYTPEERAHYGISEGLVRLSVGLEDIDDLLADVQQALKASA;
(SEQ ID NO: 147)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFXVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 148)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVXLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 149)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
XDLQALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 150)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLXALEAAMTPATRVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 151)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATXVIYFESPANPNMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRREMNALQLFSRAVSL
GDAESLAQHPASMTHSSYTPEERAHYGISEGLVRLSVGLEDIDDLLADVQQALKASA;
(SEQ ID NO: 152)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPXMHMADIAGVAKIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 153)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAXIARKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 154)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIAXKHGATVVVDNTYCTPYLQRPLELGADLV
VHSATKYLSGHGDITAGIVVGSQALVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 155)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQAXVDRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 156)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVXRIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
(SEQ ID NO: 157)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDXIRLQGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA;
and
(SEQ ID NO: 158)
HGSNKLPGFATRAIHHGYDPQDHGGALVPPVYQTATFTFPTVEYGAACFAGEQAGHFYSRISNPTLNL
LEARMASLEGGEAGLALASGMGAITSTLWTLLRPGDEVLLGNTLYGCTFAFLHHGIGEFGVKLRHVDM
ADLQALEAAMTPATRVIYFESPANPNMHMADIAGVAKIARKHGATVVVDNTYCTPYLQRPLELGADLV
VHSATKYLSGHGDITAGIVVGSQALVDRIRLXGLKDMTGAVLSPHDAALLMRGIKTLNLRMDRHCANA
QVLAEFLARQPQVELIHYPGLASFPQYTLARQQMSQPGGMIAFELKGGIGAGRRFMNALQLFSRAVSL
GDAESLAQHPASMTHSSYTPEERAHYGISEGLVRLSVGLEDIDDLLADVQQALKASA,

where the X is one of the nonnatural amino acids exemplified in the section titled “Example of nonnatural amino acid 2—by formula”.

### Embodiment 10, Limitation of Tetramer Protein, Urate Oxidase

The multimeric protein-albumin conjugate of Embodiment 5, in which the multimeric protein variant is characterized in that at least one amino acid sequence of wild-type urate oxidase derived from Arthrobacter globiformis is substituted with the nonnatural amino acid.

### Embodiment 11, Limitation of Urate Oxidase Sequence

The multimeric protein-albumin conjugate of Embodiment 10, in which a sequence of the variant subunit contained in the multimeric protein variant is selected from among the following:

(SEQ ID NO: 4)
MTATAETSTGTKVVLGQNQYGKAEVRLVKVTRNTARHEIQDLNVTSQLRGDFEAAHTAGDNAH
VVATDTQKNTVYAFARXGFATTEEFLLRLGKHFTEGFDWVTGGRWAAQQFFWDRINDHDHAFSRNKSE
VRTAVLEISGSEQAIVAGIEGLTVLKSTGSEFHGFPRDKYTTLQETTDRILATDVSARWRYNTVEVDF
DAVYASVRGLLLKAFAETHSLALQQTMYEMGRAVIETHPEIDEIKMSLPNKHHFLVDLQPFGQDNPNE
VFYAADRPYGLIEATIQREGSRADHPIWSNIAGFC;
(SEQ ID NO: 5)
MTATAETSTGTKVVLGQNQYGKAEVRLVKVTRNTARHEIQDLNVTSQLRGDFEAAHTAGDNAHVVATD
TQKNTVYAFARDGXATTEEFLLRLGKHFTEGFDWVTGGRWAAQQFFWDRINDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 6)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGXDWVTGGRWAAQQFFWDRINDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 7)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFXWVTGGRWAAQQFEWDRINDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 8)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFXWDRINDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHELVDLQPFGQDNPNEVFYAA
DRPYGLIEATIQREGSRADHPIWSNIAGFC;
(SEQ ID NO: 9)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFFWDRIXDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 10)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFFWDRINXHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 11)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFEWDRINDHDHAFSRNKSEVRTAV
LEISGXEQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 12)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFFWDRINDHDHAFSRNKSEVRTAV
LEISGSXQAIVAGIEGLTVLKSTGSEFHGFPRDKYTTLQETTDRILATDVSARWRYNTVEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 13)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFFWDRINDHDHAFSRNKSEVRTAV
LEISGSEQAIVAGIEGLTVLKSTGSEFHGFPRDKYTTLXETTDRILATDVSARWRYNTVEVDFDAVYA
SVRGLLLKAFAETHSLALQQTMYEMGRAVIETHPEIDEIKMSLPNKHHELVDLQPFGQDNPNEVFYAA
DRPYGLIEATIQREGSRADHPIWSNIAGFC;
(SEQ ID NO: 14)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFFWDRINDHDHAFSRNKSEVRTAV
LEISGSEQAIVAGIEGLTVLKSTGSEFHGFPRDKYTTLQETTDRILATDVSARWRYNTXEVDFDAVYA
SVRGLLLKAFAETHSLALQQTMYEMGRAVIETHPEIDEIKMSLPNKHHFLVDLQPFGQDNPNEVFYAA
DRPYGLIEATIQREGSRADHPIWSNIAGFC;
(SEQ ID NO: 15)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFFWDRINDHDHAFSRNKSEVRTAV
LEISGSEQAIVAGIEGLTVLKSTGSEFHGFPRDKYTTLQETTDRILATDVSARWRYNTVXVDFDAVYA
SVRGLLLKAFAETHSLALQQTMYEMGRAVIETHPEIDEIKMSLPNKHHELVDLQPFGQDNPNEVFYAA
DRPYGLIEATIQREGSRADHPIWSNIAGFC;
(SEQ ID NO: 16)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFEWDRINDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETXSLALQQTMYEMGRAVIETHPEIDEIKMSLPNKHHFLVDLQPFGQDNPNEVFYAA
DRPYGLIEATIQREGSRADHPIWSNIAGFC;
and
(SEQ ID NO: 17)
MTATAETSTGTKVVLGQNQYGKAEVRLVKVTRNTARHEIQDLNVTSQLRGDFEAAHTAGDNAHVVATD
TQKNTVYAFARDGFATTEEFLLRLGKHFTEGFDWVTGGRWAAQQFEWDRINDHDHAFSRNKSEVRTAV
LEISGSEQAIVAGIEGLTVLKSTGSEFHGFPRDKYTTLQETTDRILATDVSARWRYNTVEVDFDAVYA
SVRGLLLKAFAETHSLALQQTMYEMGRAVIETHXEIDEIKMSLPNKHHFLVDLQPFGQDNPNEVFYAA
DRPYGLIEATIQREGSRADHPIWSNIAGFC,

where the X is one of the nonnatural amino acids exemplified in the section titled “Example of nonnatural amino acid 2—by formula”.

### Embodiment 12, Pentamer Protein-Albumin Conjugate

The protein-albumin conjugate of Embodiment 1, in which the m is 5, and the multimeric protein variant contains a subunit in combination selected from among the following:
4 wild-type subunits and 1 variant subunit;
3 wild-type subunits and 2 variant subunits;
2 wild-type subunits and 3 variant subunits;
1 wild-type subunit and 4 variant subunits; and
5 variant subunits;

### Embodiment 13, Hexamer Protein-Albumin Conjugate

The protein-albumin conjugate of Embodiment 1, in which the m is 6, and the multimeric protein variant contains a subunit in combination selected from among the following:

5 wild-type subunits and 1 variant subunit;
4 wild-type subunits and 2 variant subunits;
3 wild-type subunits and 3 variant subunits;
2 wild-type subunits and 4 variant subunits;
1 wild-type subunit and 5 variant subunits; and
6 variant subunits;

### Embodiment 14, Limitation of Diene Functional Group

The multimeric protein-albumin conjugate of any one of Embodiments 1 to 13, in which the diene functional group of each nonnatural amino acid is selected from among a tetrazine functional group or a derivative thereof, and a triazine functional group or a derivative thereof.

### Embodiment 15, Limitation of Nonnatural Amino Acid 1

The multimeric protein-albumin conjugate of Embodiment 14, in which each nonnatural amino acid is selected from the group consisting of 4-(1,2,3,4-tetrazin-3-yl) phenylalanine (frTet), 4-(6-methyl-s-tetrazin-3-yl)phenylalanine (Tet-v2.0), 3-(4-(1,2,4-triazin-6-yl)phenyl)-2-aminopropanoic acid, 2-amino-3-(4-(2-(6-methyl-1,2,4,5-tetrazin-3-yl)ethyl)phenyl)propanoic acid, 2-amino-3-(4-(6-phenyl-1,2,4,5-tetrazin-3-yl)phenyl)propanoic acid, 3-(4-((1,2,4,5-tetrazin-3-yl)amino)phenyl)-2-aminopropanoic acid, 3-(4-(2-(1,2,4,5-tetrazin-3-yl)ethyl)phenyl)-2-aminopropanoic acid, 3-(4-((1,2,4,5-tetrazin-3-yl)thio)phenyl)-2-aminopropanoic acid, 2-amino-3-(4-((6-methyl-1,2,4,5-tetrazin-3-yl)thio)phenyl)propanoic acid, 3-(4-((1,2,4,5-tetrazin-3-yl)oxy)phenyl)-2-aminopropanoic acid, 2-amino-3-(4-((6-methyl-1,2,4,5-tetrazin-3-yl)oxy)phenyl)propanoic acid, 3-(4′-(1,2,4,5-tetrazin-3-yl)-[1,1′-biphenyl]-4-yl)-2-aminopropanoic acid, 2-amino-3-(4′-(6-methyl-1,2,4,5-tetrazin-3-yl)-[1,1′-biphenyl]-4-yl)propanoic acid, 2-amino-3-(6-(6-(pyridin-2-yl)-1,2,4,5-tetrazin-3-yl)pyridin-3-yl)propanoic acid, 3-(4-(1,2,4,5-tetrazin-3-yl)phenyl)-2-aminopropanoic acid, and 2-amino-3-(4-(6-methyl-1,2,4,5-tetrazin-3-yl)phenyl)propanoic acid.

### Embodiment 16, Limitation of Nonnatural Amino Acid 2

The multimeric protein-albumin conjugate of Embodiment 14, in which each nonnatural amino acid is selected from among the following:

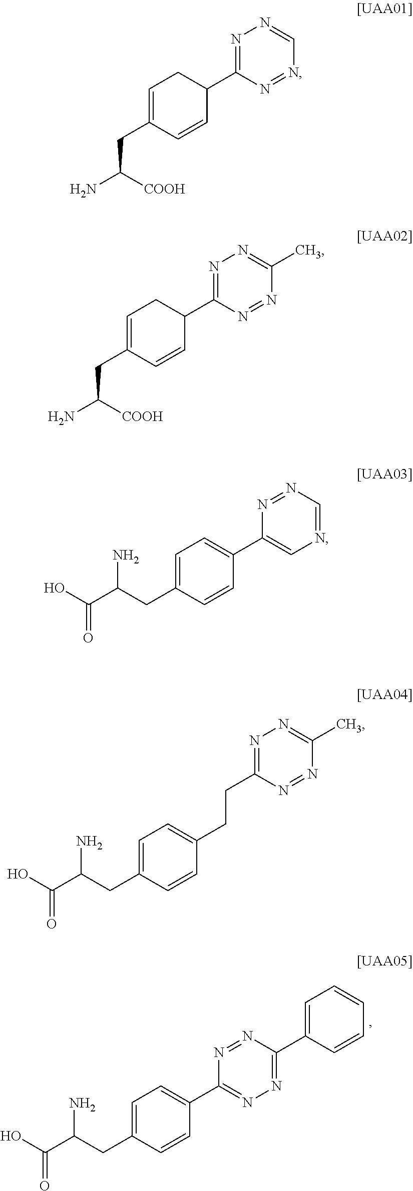 [SMILES: N[C@@H](CC1=CCC(c2nncnn2)C=C1)C(=O)O ; Cc1nnc(C2C=CC(C[C@H](N)C(=O)O)=CC2)nn1 ; NC(Cc1ccc(-c2cncnn2)cc1)C(=O)O ; Cc1nnc(CCc2ccc(CC(N)C(=O)O)cc2)nn1 ; NC(Cc1ccc(-c2nnc(-c3ccccc3)nn2)cc1)C(=O)O]

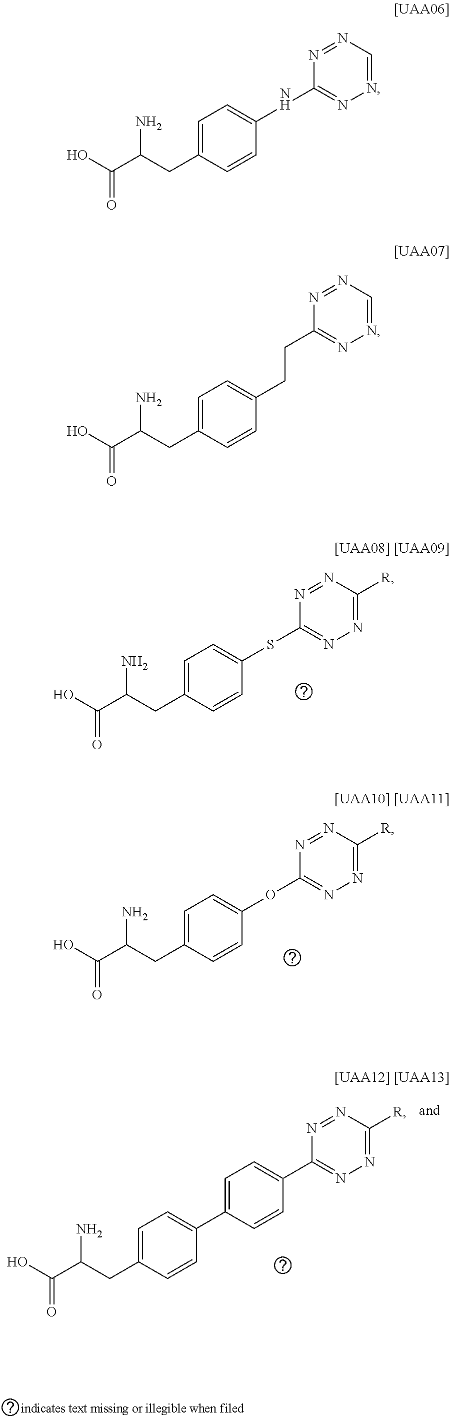

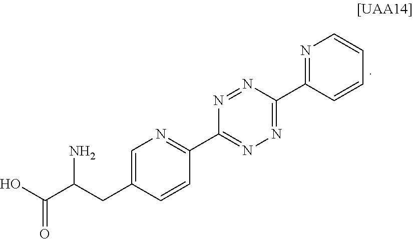 [SMILES: NC(Cc1ccc(-c2nnc(-c3ccccn3)nn2)nc1)C(=O)O]

### Embodiment 17, Limitation of Subunit Nonnatural Amino Acid Number

The multimeric protein-albumin conjugate of any one of Embodiments 1 to 16, in which the variant subunit contained in the multimeric protein contains one nonnatural amino acid.

### Embodiment 18, Identical Subunit Sequence

The multimeric protein-albumin conjugate of any one of Embodiments 1 to 17, in which all the monomer subunits contained in the multimeric protein have the same amino acid sequence.

### Embodiment 19, Maintaining Function of Multimeric Protein-Albumin Conjugate

The multimeric protein-albumin conjugate of any one of Embodiments 1 to 18, in which the multimeric protein-albumin conjugate has the same function as the wild-type multimeric protein corresponding to the multimeric protein variant.

### Embodiment 20, Substitution Site of Nonnatural Amino Acid

The multimeric protein-albumin conjugate of Embodiment 19, in which a site of the nonnatural amino acid contained in the multimeric protein variant of the multimeric protein-albumin conjugate is determined based on a molecular modeling simulation result for the multimeric protein.

### Embodiment 21, Atomic Energy and Solvent Accessibility

The multimeric protein-albumin conjugate of Embodiment 20, in which the site of the nonnatural amino acid is similar in intrinsic atomic energy to the wild-type multimeric protein and are high in solvent accessibility.

### Embodiment 22, Limitation of Rosetta Program

The multimeric protein-albumin conjugate of Embodiment 21, in which the molecular modeling simulation result is a scoring result of Rosetta molecular modeling package.

### Embodiment 23, Limitation of Multimeric Protein-Linker Junction Structure

The multimeric protein-albumin conjugate of any one of Embodiments 1 to 22, in which the multimeric protein-linker junction structure is one of the following:

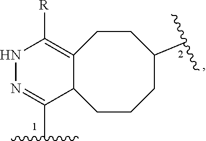

where the R is selected from among H, CH₃,

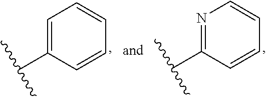 [SMILES: *c1ccccc1 ; *c1ccccn1]

the (1) is linked to the multimeric protein variant, and the (2) is linked to the anchor; and

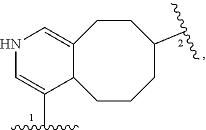 [SMILES: *C1=CNC=C2CCC(*)CCCC12]

where the (1) is linked to the multimeric protein variant, and the (2) is linked to the anchor.

### Embodiment 24, Limitation of Albumin Sequence

The multimeric protein-albumin conjugate of any one of Embodiments 1 to 23, in which the albumin is represented by a sequence selected from among the following:

(SEQ ID NO: 46)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
(SEQ ID NO: 47)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQNLIKQNCELFEQLGEYKF
QNALLVRYTKKVPQMSTPTLVEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
(SEQ ID NO: 48)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQNLIKQNCELFEQLGEYKF
QNALLVRYTKKVPQVSAPTLVEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
(SEQ ID NO: 49)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVELGMELYEYA
RRHPDYSVVLLLRLAKTYETTLEKCCAAADPHECYAKVFDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKTPVSD
RVTKCCTESLVNRRPCFSALEVDETYVPKEENARTFTFHADICTLSEKERQIKKQTALVELVKHKPKA
TKEQLKAVMDDFAAFVEKCCKADDKETCFAEEGKKLVAASQAALGL;
(SEQ ID NO: 50)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYF
RRHPDYSVVLLLRLAKTYETTLEKCCAAADPHECYAKVFDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKTPVSD
RVTKCCTESLVNRRPCFSALEVDETYVPKEFNAGTFTFHADICTLSEKERQIKKQTALVELVKHKPKA
TKEQLKAVMDDFAAFVEKCCKADDKETCFAEEGKKLVAASQAALGL;
(SEQ ID NO: 51)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVEDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAAMDDFAAFVEKCCKADDKETCFAEEGKKLVAASQAALGL;
(SEQ ID NO: 52)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMELYEYA
RRHPDYSVVLLLRLAKTYETTLEKCCAAADPHECYAKVEDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGYKLVAASQAALGL;
(SEQ ID NO: 53)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADEVESKDVCKNYAEAKDVFLGMFLYEYA
RRHPDYSVVLLLRLAKTYETTLEKCCAAADPHECYAKVFDEFKPLVEEPQNLIKQNCELFEQLGEYKF
QNALLVRYTKKVPQVSTPTLIEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
(SEQ ID NO: 54)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVEDEFKPLVEEPQNLIKQNCELFEQLGEYKF
QNALLVRYTKKVPQVSTPTLVEVSRDLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
(SEQ ID NO: 55)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVFDEFKPLVEEPQNLIKQNCELFEQLGEYKF
QNALLVRYTKKVPQVSTPTLVEVSRNLGKVGSKCCKHPEAKRMPCVEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
(SEQ ID NO: 56)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVELGMFLYEYA
RRHPDYSVVLLLRLAKTYETTLEKCCAAADPHECYAKVEDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKMPVSD
RVTKCCTESLVNRRPCFSALEVDETYVPKEFNAETFTFHADICTLSEKERQIKKQTALVELVKHKPKA
TKEQLKAVMDDFAAFVEKCCKADDKETCFAEEGKKLVAASQAALGL;
and
(SEQ ID NO: 57)
DAHKSEVAHRFKDLGEENFKALVLIAFAQYLQQCPFEDHVKLVNEVTEFAKTCVADESAENCD
KSLHTLFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVFLGMFLYEYA
RRHPDYSVVLLLRLAKTYETTLEKCCAAADPHECYAKVEDEFKPLVEEPQNLIKQNCELFEQLGEYKF
QNALLVRYTKKVPQVSTPTLVEVSRNLGKVGSKCCKHPEAKRMPCAEDYLSVVLNQLCVLHEKTPVSD
RVTKCCTESLVNRRPCFSALEVDETYVPKEFNAETFTFHADICTLSEKERQIKKQTALVELVKHKPKA
TKEQLKAVMDDFTAFVEKCCKADDKETCFAEEGKKLVAASQAALGL.

### Embodiment 25, Albumin-Linker Junction, Limitation of Reactive Group

The multimeric protein-albumin conjugate of any one of Embodiments 1 to 24, in which the albumin-linker junction has a junction structure formed through a reaction between a thiol moiety of the albumin and a thiol reactive moiety linked to the anchor.

### Embodiment 26, Albumin-Linker Junction, Limitation of Sequence and Junction Site

The multimeric protein-albumin conjugate of Embodiment 25, in which a sequence of the albumin is selected from among SEQ ID Nos: 46 to 57, and the albumin-linker junction has a junction structure formed through a reaction between the thiol moiety of the 34th cysteine residues in the albumin sequence and the thiol reactive moiety linked to the anchor.

### Embodiment 27, Limitation of Albumin-Linker Junction Structure

The multimeric protein-albumin conjugate of any one of Embodiments 1 to 26, in which the albumin-linker junction is any one selected from among the following:

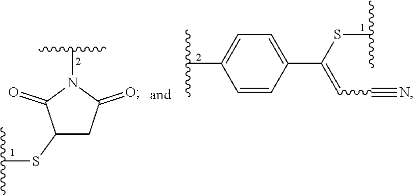 [SMILES: *SC1CC(=O)N(*)C1=O ; *SC(=CC#N)c1ccc(*)cc1]

where the (1) is linked to albumin, and the (2) is linked to the anchor of the linker.

### Embodiment 28, Limitation of Anchor

The multimeric protein-albumin conjugate of any one of Embodiments 1 to 27, in which the anchor is any one selected from among the following:

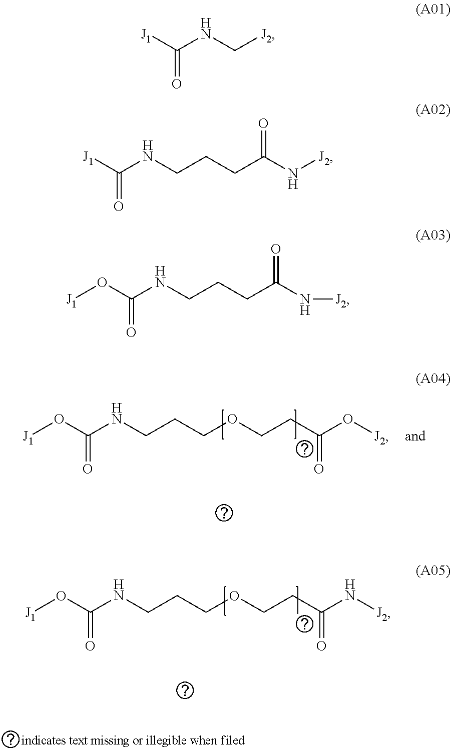

where the J₁ the multimeric protein-linker junction, and the J₂ is the albumin-linker junction.

### Multimeric Protein-Albumin Conjugate 2

### Embodiment 29, Multimeric Protein-Albumin Conjugate in View of Subunit

A multimeric protein-albumin conjugate containing the following:
n albumin-subunit conjugates and (m-n) variant subunits,
where the m is an integer of 2 or more and 6 or less, and the n is an integer of 1 or more and m or less,
in which the albumin-subunit conjugate is represented by the following Structural Formula 2:

p′-J₁-A-J₂-HAS, [Structural Formula 2]
where the p′ is a variant subunit, the J₁ is a multimeric protein-linker junction, the A is an anchor, the J₂ is an albumin-linker junction, and the HAS is human serum albumin,
in which the n albumin-subunit conjugates and the (m-n) variant subunits are oligomerized to form a multimeric protein-albumin conjugate.

### Embodiment 30, Limitation of Multimeric Protein-Linker Junction

The multimeric protein-albumin conjugate of Embodiment 29, in which the multimeric protein-linker junctions are each independently selected from among the following:

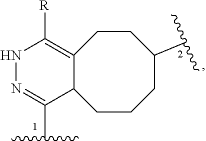

where the R is selected from among H, CH₃,

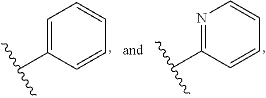 [SMILES: *c1ccccc1 ; *c1ccccn1]

the (1) is linked to a multimeric protein variant, and the (2) is linked to the anchor of a linker; and

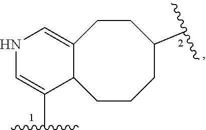 [SMILES: *C1=CNC=C2CCC(*)CCCC12]

where the (a) is linked to the multimeric protein variant, and the (2) is linked to the anchor of the linker.

### Embodiment 31, Limitation of A

The multimeric protein-albumin conjugate of any one of Embodiments 29 to 31, in which the anchors are each independently selected from among the following:

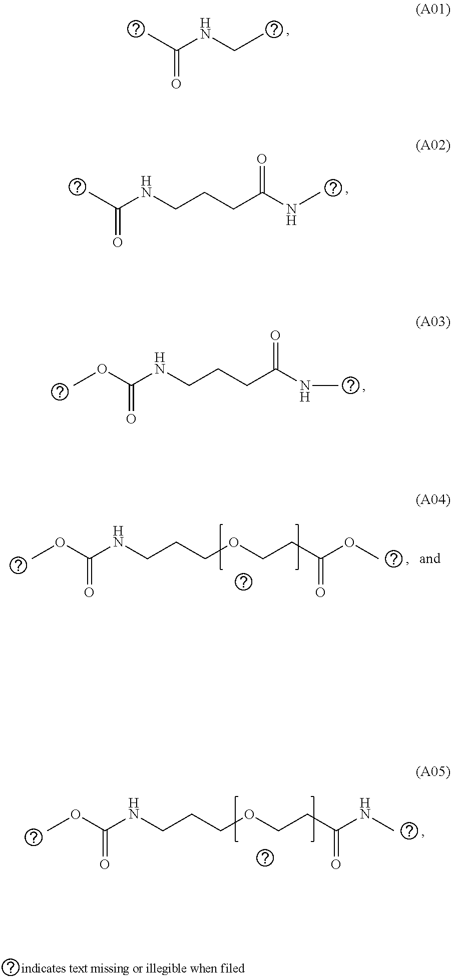

where the J₁ is the multimeric protein-linker junction, and the J₂ is the albumin-linker junction.

### Embodiment 32, Limitation of Albumin-Linker Junction

The multimeric protein-albumin conjugate of any one of Embodiments 29 to 31, in which the albumin-linker junctions are each independently selected from among the following:

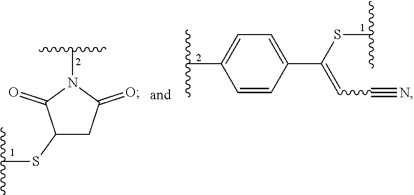 [SMILES: *SC1CC(=O)N(*)C1=O ; *SC(=CC#N)c1ccc(*)cc1]

where the (1) is linked to albumin, and the (2) is linked to the anchor of the linker.

### Embodiment 33, Limitation of Albumin

The multimeric protein-albumin conjugate of any one of Embodiments 29 to 32, in which the albumin is represented by a sequence selected from among SEQ ID Nos: 46 to 57.

### Embodiment 34, Limitation of Urate Oxidase

The multimeric protein-albumin conjugate of any one of Embodiments 29 to 33, in which each of the variant subunits contained in the n albumin-subunit conjugates and the (m-n) variant subunits is a urate oxidase variant subunit independently selected from among SEQ ID NOs: 4 to 17.

### Embodiment 35, Limitation of Asparaginase

The multimeric protein-albumin conjugate of any one of Embodiments 29 to 33, in which each of the variant subunits contained in the n albumin-subunit conjugates and the (m-n) variant subunits is an asparaginase variant subunit independently selected from among SEQ ID NOs: 18 to 40 and 138.

### Embodiment 36, Limitation of Methioninase

The multimeric protein-albumin conjugate of any one of Embodiments 29 to 33, in which each of the variant subunits contained in the n albumin-subunit conjugates and the (m-n) variant subunits is a methioninase variant subunit independently selected from among SEQ ID NOs: 41 to 45 and 139 to 158.

### Multimeric Protein Variant

### Embodiment 37, Multimeric Protein Variant

A multimeric protein variant containing at least one nonnatural amino acid,
in which each nonnatural amino acid has a tetrazine functional group or a triazine functional group.

### Embodiment 38, Containing Subunit

The multimeric protein variant of Embodiment 37,
in which the multimeric protein variant is a multimer formed by oligomerization of n variant subunits and (m-n) wild-type subunits,
where the n is an integer of 1 or more and m or less, and the m is an integer of 2 or more and 6 or less.

### Embodiment 39, Limitation of Multimeric Protein, Urate Oxidase

The multimeric protein variant of Embodiment 37 or 38, in which the m is 4, the multimeric protein is urate oxidase derived from Arthrobacter globiformis, the wild-type subunit is represented by an amino acid sequence of SEQ ID No: 1, and each variant subunit is characterized in that at least one amino acid sequence of the wild-type subunit is substituted with the nonnatural amino acid.

### Embodiment 40, Limitation of Multimeric Protein, Asparaginase

The multimeric protein variant of Embodiment 37 or 38, in which the m is 4, the multimeric protein is asparaginase derived from Erwinia chrysanthemi, the wild-type subunit is represented by an amino acid sequence of SEQ ID No: 2, and each variant subunit is characterized in that at least one amino acid sequence of the wild-type subunit is substituted with the nonnatural amino acid.

### Embodiment 41, Limitation of Multimeric Protein, Methioninase

The multimeric protein variant of Embodiment 37 or 38, in which the m is 4, the multimeric protein is methioninase derived from Pseudomonas putida, the wild-type subunit is represented by an amino acid sequence of SEQ ID No: 3, and each variant subunit is characterized in that at least one amino acid sequence of the wild-type subunit is substituted with the nonnatural amino acid.

### Embodiment 42, Limitation of Substitution Site

The multimeric protein variant of any one of Embodiments 37 to 41, in which each variant subunit has a form in which an amino acid positioned at one or more substitution-suitable sites in the wild-type subunit is substituted with the nonnatural amino acid, and
the substitution-suitable sites hardly affect the function and structure of the urate oxidase subunit and are high in solvent accessibility.

### Embodiment 43, Limitation of Substitution Site Determination Method

The multimeric protein variant of Embodiment 42, the substitution-suitable site is determined with reference to a molecular modeling simulation result, preferably, a scoring result of Rosetta molecular modeling package.

### Embodiment 44, Urate Oxidase, Sequence Selection

The multimeric protein variant of Embodiment 39, in which each variant subunit has a sequence independently selected from among SEQ ID Nos: 4 to 17.

### Embodiment 45, Urate Oxidase, Identical Sequence

The multimeric protein variant of Embodiment 39, in which the multimeric protein variant contains four variant subunits, and the variant subunits have the same sequence selected from among SEQ ID Nos: 4 to 17.

### Embodiment 46, Asparaginase, Sequence Selection

The multimeric protein variant of Embodiment 40, in which each variant subunit has a sequence independently selected from among SEQ ID Nos: 18 to 40 and 138.

### Embodiment 47, Asparaginase, Identical Sequence

The multimeric protein variant of Embodiment 40, in which the multimeric protein variant contains four variant subunits, and the variant subunits have the same sequence selected from among SEQ ID Nos: 18 to 40 and 138.

### Embodiment 48, Methioninase, Sequence Selection

The multimeric protein variant of Embodiment 41, in which each variant subunit has a sequence independently selected from among SEQ ID Nos: 41 to 45 and 139 to 158.

### Embodiment 49, Methioninase, Identical Sequence

The multimeric protein variant of Embodiment 41, in which the multimeric protein variant contains four variant subunits, and the variant subunits have the same sequence selected from among SEQ ID Nos: 41 to 45 and 139 to 158.

Multimeric Protein Variant-Expressing Vector

### Embodiment 50, Multimeric Protein Variant Expression Vector

A vector capable of expressing the multimeric protein variant of any one of Embodiments 37 to 49, in which a portion corresponding to the nonnatural amino acid contained in the urate oxidase variant is encoded with one selected from among an amber codon (5′-UAG-3′), an ocher codon (5′-UAA-3′), and an opal codon (5′-UGA-3′).

### Embodiment 51, Limitation of Multimeric Protein Variant Expression Vector Sequence, Urate Oxidase Variant

The vector of Embodiment 50, in which the vector has a nucleic acid sequence selected from among SEQ ID Nos: 58 to 71.

### Embodiment 52, Limitation of Multimeric Protein Variant-Expressing Vector Sequence, Asparaginase Variant

The vector of Embodiment 50, in which the vector has a nucleic acid sequence selected from among SEQ ID Nos: 100 to 104.

### Embodiment 53, Limitation of Multimeric Protein Variant Expression Vector Sequence, Methioninase Variant

The vector of Embodiment 50, in which the vector has a nucleic acid sequence selected from among SEQ ID Nos: 117 to 104.

Multimeric Protein Variant Preparation Method

### Embodiment 54, Preparation Method of Multimeric Protein Variant

A preparation method of a multimeric protein variant, the method including the following:
constructing a vector capable of expressing an orthogonal tRNA/synthetase pair and a cell line containing a multimeric protein variant expression vector,
in which the vector capable of expressing the orthogonal tRNA/synthetase pair is capable of expressing exogenous suppressor tRNA and exogenous tRNA synthetase,
the exogenous suppressor tRNA is capable of recognizing a specific stop codon,
the exogenous tRNA synthetase is capable of recognizing a nonnatural amino acid having a tetrazine functional group and/or triazine functional group so as to be linked to the exogenous suppressor tRNA, and
the multimeric protein variant expression vector is capable of expressing the multimeric protein variant of any one of Embodiments 37 to 49, where the site corresponding to the nonnatural amino acid contained in the multimeric protein variant is encoded with the specific stop codon; and
culturing the cell line in a medium containing at least one type of nonnatural amino acid having a tetrazine functional group and/or triazine functional group.

### Embodiment 55, Limitation of Stop Codon

The method of Embodiment 54, in which the specific stop codon is selected from among an amber codon (5′-UAG-3′), an ocher codon (5′-UAA-3′), and an opal codon (5′-UGA-3′).

### Embodiment 56, Cell Line Variation

The method of Embodiment 55, in which the cell line is a cell line where a release factor responsible for recognizing the specific stop codon is inactivated.

### Embodiment 57, Limitation of Cell Line

The method of Embodiment 56, in which the cell line is E. coli C321.ΔA.exp (Addgene, ID:49018).

### Embodiment 58, Limitation of Orthogonal tRNA/Synthetase Pair

The method of Embodiment 54, in which the orthogonal tRNA/synthetase pair is suppressor tRNA (MjtRNATyrCUA) derived from Methanococcus jannaschii, and tyrosyl-tRNA synthetase (MjTyrRS) derived from Methanococcus jannaschii.

### Embodiment 59, Limitation of Vector Expressing Orthogonal tRNA/Synthetase Pair

The method of Embodiment 58, in which the vector capable of expressing the orthogonal tRNA/synthetase pair is pDule_C11 disclosed in Yang et.al (Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464).

### Multimeric Protein-Albumin Conjugate Preparation Method 1

### Embodiment 60, Albumin-Linker Conjugation First

A preparation (manufacturing) method of a multimeric protein-albumin conjugate, the method including the following:

reacting albumin and a linker,
in which the linker contains a dienophile functional group, an anchor, and a thiol reactive moiety, and
the thiol reactive moiety of the linker is bound to a thiol moiety of the albumin through a reaction to form an albumin-linker conjugate; and
reacting the albumin-linker conjugate and a multimeric protein variant,
in which the multimeric protein variant has a form in which n or more amino acids in a sequence of a wild-type multimeric protein composed of m subunits are substituted with a nonnatural amino acid having a diene functional group,
where the m is an integer of 2 or more and 6 or less, and the n is an integer of 1 or more,
the diene functional group of the multimeric protein variant is bound to the dienophile functional group in the linker of the albumin-linker conjugate through an inverse electron-demand Diels-Alder (IEDDA) reaction to form a multimeric protein-albumin conjugate, and
the multimeric protein-albumin conjugate has a form in which at least one albumin is conjugated to the multimeric protein variant through the linker.

### Embodiment 61, Multimeric Protein-Albumin Conjugation First

A preparation method of a multimeric protein-albumin conjugate, the method including the following:
reacting a multimeric protein variant and a linker,
in which the multimeric protein variant has a form in which n or more amino acids in a sequence of a wild-type multimeric protein composed of m subunits are substituted with a nonnatural amino acid having a diene functional group,
where the m is an integer of 2 or more and 6 or less, and the n is an integer of 1 or more
the linker contains a dienophile functional group, an anchor, a thiol reactive moiety,
the diene functional group of the multimeric protein variant is bound to the dienophile functional group of the linker through an inverse electron-demand Diels-Alder (IEDDA) reaction to form a multimeric protein-linker conjugate, and
the multimeric protein-linker conjugate has a form in which at least one linker is conjugated to the multimeric protein variant; and
reacting the multimeric protein-linker conjugate and albumin,
in which the thiol reactive moiety contained in the linker of the multimeric protein-linker conjugate is bound to a thiol moiety of the albumin through a reaction to form a multimeric protein-albumin conjugate, and
the multimeric protein-albumin conjugate has a form in which at least one albumin is conjugated to the multimeric protein-linker conjugate.

### Embodiment 62, Multimeric Protein, Linker, and Albumin Simultaneously

A preparation method of a multimeric protein-albumin conjugate, the method including the following:
reacting a multimeric protein variant, a linker, and albumin,
in which the multimeric protein variant is has a form in which n or more amino acids in a sequence of a wild-type multimeric protein composed of m subunits are substituted with a nonnatural amino acid containing a diene functional group,
where the m is an integer of 2 or more and 6 or less, and the n is an integer of 1 or more,
the linker contains a dienophile functional group, an anchor, a thiol reactive moiety,
a thiol moiety of the albumin and the thiol reactive moiety of the linker react for conjugation,
where a diene functional group in the nonnatural amino acid of the multimeric protein variant and an IEDDA reactive group of the linker react for conjugation, and
the multimeric protein-albumin conjugate is formed as a result of the reaction.

### Embodiment 63, Limitation of Multimeric Protein Variant

The method of any one of Embodiments 60 to 62, in which the multimeric protein variant is a tetramer that four multimeric protein variant subunits are oligomerized, and compared to a wild-type multimeric protein subunit, the multimeric protein variant subunit has a form in which at least one amino acid is substituted with the nonnatural amino acid having a diene functional group.

### Embodiment 64, Limitation of Diene Functional Group of Nonnatural Amino Acid

The method of any one of Embodiments 60 to 62, in which the diene functional group of the nonnatural amino acid is a tetrazine functional group or a triazine functional group.

### Embodiment 65, Limitation of Dienophile Functional Group

The method of any one of Embodiments 60 to 62, in which the dienophile functional group of the linker is selected from among trans-cyclooctene or a derivative thereof.

### Embodiment 66, Limitation of Thiol Reactive Moiety

The method of any one of Embodiments 60 to 62, in which the thiol reactive moiety is selected from among maleimide or a derivative thereof, and 3-arylpropiolonitriles or a derivative thereof.

### Embodiment 67, Reactant Markush

The method of any one of Embodiments 60 to 62,
in which the multimeric protein variant is a multimeric protein formed by oligomerization of m sequences each independently selected from among SEQ ID Nos: 4 to 63, and X of the selected sequence is each independently selected from among the nonnatural amino acids exemplified in the section titled “Example of nonnatural amino acid 2—by formula”,
the albumin is represented by an amino acid sequence selected from among SEQ ID Nos: 46 to 57,
the thiol moiety of the albumin is a thiol moiety of the 34th cysteine in an amino acid sequence selected from among SEQ ID Nos: 46 to 57, and
the linker is one linker exemplified in the section titled “Examples of linker”.

### Multimeric Protein-Albumin Conjugate Preparation Method 2

### Embodiment 68, Novel Process

A preparation method of multimeric protein-albumin conjugate, the method including the following:
disrupting a cell,
in which the cell contains a multimeric protein variant containing a nonnatural amino acid having at least one diene functional group;
adding an albumin-linker conjugate to the cell disruption product,
in which the albumin-linker conjugate contains a dienophile functional group, and
the diene functional group of the multimeric protein variant is bound to the dienophile functional group in the linker of the albumin-linker conjugate through the inverse electron-demand Diels-Alder (EDDA) reaction to form a multimeric protein-albumin conjugate; and
obtaining the multimeric protein-albumin conjugate.

### Embodiment 69, Additional Pretreatment Process after Cell Disruption

The method of Embodiment 68, further including a pretreatment process after disrupting the cell and before adding the albumin-linker conjugate, in which a cell disruption product is generated as a result of the pretreatment process.

### Embodiment 70, Limitation of Pretreatment Process

The method of Embodiment 69, in which the pretreatment process is selected from among centrifugation, supernatant collection, filtering, chromatographic purification, and/or combinations thereof.

### Embodiment 71, Limitation of Additional Cell Component

The method of any one of Embodiments 68 to 70, in which the cell further contains a component selected from among the following:
exogenous suppressor tRNA; exogenous tRNA synthetase; and a vector encoding the multimeric protein variant.

### Embodiment 72, Limitation of Cell Type

The method of any one of Embodiments 68 to 70, in which the cell is selected from among the following:

Escherichia genus; Erwinia genus; Serratia genus; Providencia genus; Corynebacterium genus; Pseudomonas genus; Leptospira genus; Salmonellar genus; Brevibacterium genus; Hyphomonas genus; Chromobacterium genus; Nocardia genus; fungi; and yeasts.

### Embodiment 73, Limitation of Cell Function

The method of any one of Embodiments 68 to 70, in which the cell has a form in which a release factor responsible for recognizing a stop codon selected from among an amber codon (5′-UAG-3′), an ocher codon (5′-UAA-3′), and an opal codon (5′-UGA-3′) and stopping translation is inactivated.

### Embodiment 74, Limitation of Nonnatural Amino Acid Diene Functional Group

The method of any one of Embodiments 68 to 73, in which the diene functional group of the nonnatural amino acid is a tetrazine functional group or triazine functional group.

### Embodiment 75, Limitation of Dienophile Functional Group

The method of any one of Embodiments 68 to 74, in which the dienophile functional group of the albumin-linker conjugate is selected from among trans-cyclooctene and a derivative thereof.

### Embodiment 76, Limitation of Albumin-Linker

The method of any one of Embodiments 68 to 75, in which the albumin-linker conjugate is represented by Formula 3:

I-A-J-HAS, [Formula 3]
where I is an IEDDA reactive group, one of those described in the section titled “Linker structure 1—IEDDA reactive group”,
A is an anchor, one of those described in the section titled “Anchor”,
J is an albumin-linker junction, one of those described in the section titled “Albumin-linker junction”, and
HAS is albumin, one of those described in the section titled “Albumin”.

### Embodiment 77, Limitation of Urate Oxidase

The method of any one of Embodiments 68 to 76, in which the cell contains a urate oxidase subunit selected from among SEQ ID Nos: 4 to 17.

### Embodiment 78, Limitation of Asparaginase

The method of any one of Embodiments 68 to 76, in which the cell contains an asparaginase subunit selected from among SEQ ID Nos: 18 to 40 and 138.

### Embodiment 79, Limitation of Methioninase

The method of any one of Embodiments 68 to 76, in which the cell contains a methioninase subunit selected from among SEQ ID Nos: 41 to 45 and 139 to 158.

### Embodiment 80, Limitation of End Product

The method of any one of Embodiments 68 to 79, in which in the obtaining process of the multimeric protein-albumin conjugate, the multimeric protein-albumin conjugate of any one of Embodiments 1 to 36 is obtained.

### Pharmaceutical Composition Containing Multimeric Protein-Albumin Conjugate

### Embodiment 81, Pharmaceutical Composition Containing Urate Oxidase-Albumin Conjugate

A pharmaceutical composition for preventing or treating uric acid-related diseases, the composition containing the following:
the multimeric protein-albumin conjugate of any one of Embodiments 14 to 28 and 34,
in which the multimeric protein variant is characterized in that at least one amino acid sequence of wild-type urate oxidase derived from Arthrobacter globiformis is substituted with the nonnatural amino acid, and a sequence of a variant subunit contained in the multimeric protein variant is characterized by being selected from among SEQ ID Nos: 4 to 17; and
a pharmaceutically carrier.

### Embodiment 82, Limitation of Indication

The composition of Embodiment 81, in which the uric acid-related disease is any one of hyperuricemia, acute gouty arthritis, intermittent gout, chronic nodular gout, chronic kidney disease, and/or tumor lysis syndrome (TLS).

### Embodiment 83, Pharmaceutical Composition Containing Asparaginase-Albumin Conjugate

A pharmaceutical composition for preventing or treating acute lymphoblastic leukemia, the composition containing the following:
the multimeric protein-albumin conjugate of any one of Embodiments 14 to 28 and 35,
in which the multimeric protein variant is characterized in that at least one amino acid sequence of wild-type asparaginase derived from Erwinia chrysanthemi is substituted with the nonnatural amino acid, and a sequence of a variant subunit contained in the multimeric protein variant is characterized by being selected from among SEQ ID Nos: 18 to 40 and 138; and
a pharmaceutically acceptable carrier.

### Embodiment 84, Pharmaceutical Composition Containing Methioninase-Albumin Conjugate

A pharmaceutical composition for preventing or treating cancer, the composition containing the following:
the multimeric protein-albumin conjugate of any one of Embodiments 14 to 28 and 36,
in which the multimeric protein variant is characterized in that at least one amino acid sequence of wild-type methioninase derived from Pseudomonas putida is substituted with the nonnatural amino acid, and a sequence of a variant subunit contained in the multimeric protein variant is characterized by being selected from among SEQ ID Nos: 41 to 45 and 139 to 158; and
a pharmaceutically acceptable carrier.

### Embodiment 85, Limitation of Pharmaceutically Acceptable Carrier

The composition of any one of Embodiments 9 to 84, in which the pharmaceutically acceptable carrier contains at least one of the following:
binders such as lactose, saccharose, sorbitol, mannitol, starch, amylopectin, cellulose, or gelatin; excipients such as dicalcium phosphate and the like; disintegrants such as corn starch or sweet potato starch; lubricants such as magnesium stearate, calcium stearate, sodium stearyl fumarate, or polyethylene glycol wax; sweetener; air freshener; syrup; liquid carriers such as fatty oils; sterile aqueous solution; propylene glycol; polyethylene glycol; injectable esters such as ethyl oleate; suspending agent; emulsion; freeze-dried preparations; external preparations; stabilizer; buffer; animal oil; vegetable oil; wax; paraffin; starch; tragacanth; cellulose derivatives; polyethylene glycol; silicon; bentonite; silica; talc; and zinc oxide.

### Treatment Method Using Multimeric Protein-Albumin Conjugate

### Embodiment 86, Typical Treatment Method

A preventive or therapeutic method for disease, the method including the following:
administering the pharmaceutical composition of any one of Embodiments 81 to 85 to a patient's body.

### Embodiment 87, Use of Urate Oxidase-Albumin Conjugate

The method of Embodiment 86, in which the disease is a uric acid-related disease, the multimeric protein variant contained in the pharmaceutical composition is characterized in that at least one amino acid sequence of wild-type urate oxidase derived from Arthrobacter globiformis is substituted with the nonnatural amino acid, and a sequence of a variant subunit contained in the multimeric protein variant is selected from among SEQ ID Nos: 4 to 17.

### Embodiment 88, Detailed Limitation of Indication

The method of Embodiment 87, in which the uric acid-related disease is any one of hyperuricemia, acute gouty arthritis, intermittent gout, chronic nodular gout, chronic kidney disease, and/or tumor lysis syndrome (TLS).

### Embodiment 89, Use of Asparaginase-Albumin Conjugate

The method of Embodiment 86, in which the disease is acute lymphoblastic leukemia, the multimeric protein variant contained in the pharmaceutical composition is characterized in that at least one amino acid sequence of wild-type asparaginase derived from Erwinia chrysanthemi is substituted with the nonnatural amino acid, and a sequence of a variant subunit contained in the multimeric protein variant is selected from among SEQ ID Nos: 18 to 40 and 138.

### Embodiment 90, Use of Methioninase-Albumin Conjugate

The method of Embodiment 86, in which the disease is cancer, the multimeric protein variant contained in the pharmaceutical composition is characterized in that at least one amino acid sequence of wild-type methioninase derived from Pseudomonas putida is substituted with the nonnatural amino acid, and a sequence of a variant subunit contained in the multimeric protein variant is selected from among SEQ ID Nos: 41 to 45 and 139 to 158.

### Embodiment 91, Limitation of Administration Method

The method of any one of Embodiments 86 to 90, in which the administration method is selected from among oral administration, parenteral administration, intravenous administration, intravenous infusion, intraperitoneal administration, intramuscular administration, transdermal administration, and subcutaneous administration.

### Embodiment 92, Limitation of Dosage

The method of any one of Embodiments 86 to 91, in which the pharmaceutical composition is administered to the patient's body at a dosage of 0.01 mg/kg to 1000 mg/kg.

### Embodiment 93, Limitation of Administration Cycle 1

The method of any one of Embodiments 86 to 92, in which the pharmaceutical composition is administered once a day.

### Embodiment 94, Limitation of Administration Cycle 2

The method of any one of Embodiments 86 to 92, in which the pharmaceutical composition is administered two or more times a day.

### Use of Multimeric Protein-Albumin Conjugate

### Embodiment 95, Use for Manufacturing Therapeutic Agent for Uric Acid-Related Diseases

A use of the multimeric protein-albumin conjugate of any one of Embodiments 14 to 28 and 34 for manufacturing a therapeutic agent for uric acid-related diseases,
in which the multimeric protein variant is characterized in that at least one amino acid sequence of wild-type urate oxidase derived from Arthrobacter globiformis is substituted with the nonnatural amino acid, and a sequence of a variant subunit contained in the multimeric protein variant is selected from among SEQ ID Nos: 4 to 17.

### Embodiment 96, Use for Manufacturing Therapeutic Agents for Acute Lymphoblastic Leukemia

A use of the multimeric protein-albumin conjugate of any one of Embodiments 14 to 28 and 35 for manufacturing a therapeutic agent for acute lymphoblastic leukemia,
in which the multimeric protein variant is characterized in that at least one amino acid sequence of wild-type asparaginase derived from Erwinia chrysanthemi is substituted with the nonnatural amino acid, and a sequence of a variant subunit contained in the multimeric protein variant is selected from among SEQ ID Nos: 18 to 40 and 138.

### Embodiment 97, Use for Manufacturing Therapeutic Agents for Cancer

A use of the multimeric protein-albumin conjugate of any one of Embodiments 14 to 28 and 36 for manufacturing a therapeutic agent for cancer,
in which the multimeric protein variant contained in the pharmaceutical composition is characterized in that at least one amino acid sequence of wild-type methioninase derived from Pseudomonas putida is substituted with the nonnatural amino acid, and a sequence of a variant subunit contained in the multimeric protein variant is selected from among SEQ ID Nos: 41 to 45 and 139 to 158.
   Embodiments Related to Arthrobacter globiformis-Derived Urate Oxidase-Albumin Conjugate

### Embodiment 98, Urate Oxidase-Albumin Conjugate

A urate oxidase-albumin conjugated represented by Formula 1:

Uox-[J₁-A-J₂-HSA]ₙ, [Formula 1]
where the Uox means a urate oxidase variant, the J₁ means a urate oxidase-linker junction, the A means an anchor, the J₂ means an albumin-linker junction, and HAS means human serum albumin,
in which the n is 3 or 4,
the urate oxidase variant is a tetramer that four urate oxidase variant subunits are oligomerized,
each urate oxidase variant subunit has a peptide sequence each independently selected from among SEQ ID Nos: 4 to 17 or a sequence 90% or more identical to the selected peptide sequence,
where the sequence of SEQ ID Nos: 4 to 17 contains a nonnatural amino acid X while the nonnatural amino acid is 4-(1,2,3,4-tetrazine-3-yl) phenylalanines (frTet),
the urate oxidase-linker junction has a junction structure formed through the inverse electron-demand Diels-Alder (IEDDA) reaction between a tetrazine moiety of the nonnatural amino acid contained in the urate oxidase variant and a trans-cyclooctene moiety linked to the anchor,
the urate oxidase-linker junction is represented by the following formula,

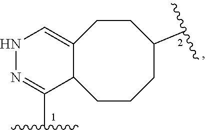 [SMILES: *C1=NNC=C2CCC(*)CCCC21]

where the (1) is liked to residues of the nonnatural amino acid, and the (2) is linked to the anchor,
in which the albumin-linker junction has a junction structure formed through a reaction between a thiol moiety of the albumin and a thiol reactive moiety of the anchor

### Embodiment 99, Urate Oxidase-Albumin Conjugate, Limitation of Urate Oxidase Sequence

The conjugate of Embodiment 98, in which the urate oxidase variant contains four urate oxidase variant subunits of SEQ ID No: 15, and the nonnatural amino acid X of the SEQ ID No: 15 is frTet.

### Embodiment 100, Urate Oxidase-Albumin Conjugate, Limitation of Albumin-Linker Junction

The conjugate of Embodiment 98 or 99, in which the albumin-linker junction is selected from among the following:

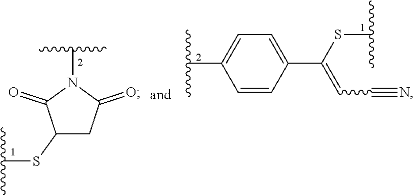 [SMILES: *SC1CC(=O)N(*)C1=O ; *SC(=CC#N)c1ccc(*)cc1]

where (1) is linked to the albumin, and (2) is linked to the anchor.

### Embodiment 101, Urate Oxidase-Albumin Conjugate, Limitation of Anchor

The conjugate of any one of Embodiments 98 to 100, in which the anchor is selected from among the following:

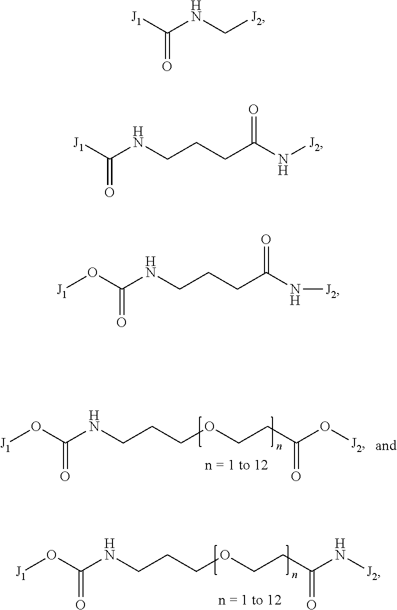

where the J₁ is the urate oxidase-linker junction, and the J₂ is the albumin-linker junction.

### Embodiment 102, Urate Oxidase-Albumin Conjugate, Limitation of Albumin

The conjugate of Embodiment 1, in which the albumin has a sequence selected from among SEQ ID Nos: 46 to 57 or a sequence 90% or more identical to the selected sequence.

### Embodiment 103, Preparation Method of Urate Oxidase-Albumin Conjugate

A preparation method of a urate oxidase-albumin conjugate, the method including the following:
reacting albumin and a linker,
in which the linker contains a dienophile functional group, an anchor, and a thiol reactive moiety,
the dienophile functional group is trans-cyclooctene or a derivative thereof,
the thiol reactive moiety is selected from among maleimide or a derivative thereof and 3-arylpropiolonitriles or a derivative thereof, and
the thiol reactive moiety of the linker is bound to a thiol moiety of the albumin through a reaction to form an albumin-linker conjugate; and
reacting the albumin-linker conjugate and a urate oxidase variant,
in which the urate oxidase variant is a tetramer that four urate variant subunits are oligomerized,
the urate oxidase variant subunits each independently represented by a sequence selected from among SEQ ID Nos: 4 to 17 or a sequence 90% or more identical to the selected sequence,
X of the SEQ ID Nos: 4 to 17 is 4-(1,2,3,4-tetrazine-3-yl) phenylalanines (frTet), which is a nonnatural amino acid,
the urate oxidase variant contains four frTet,
a tetrazine functional group in residues of the frTet is bound to the dienophile functional group of the linker through an inverse electron-demand Diels-Alder (IEDDA) reaction to form a urate oxidase-albumin conjugate, and
the urate oxidase-albumin conjugate has a form in which three or more albumins are conjugated to the urate oxidase variant through the linker.

### Embodiment 104, Preparation Method, Limitation of Linker

The method of Embodiment 103, in which the linker is selected from among the following:

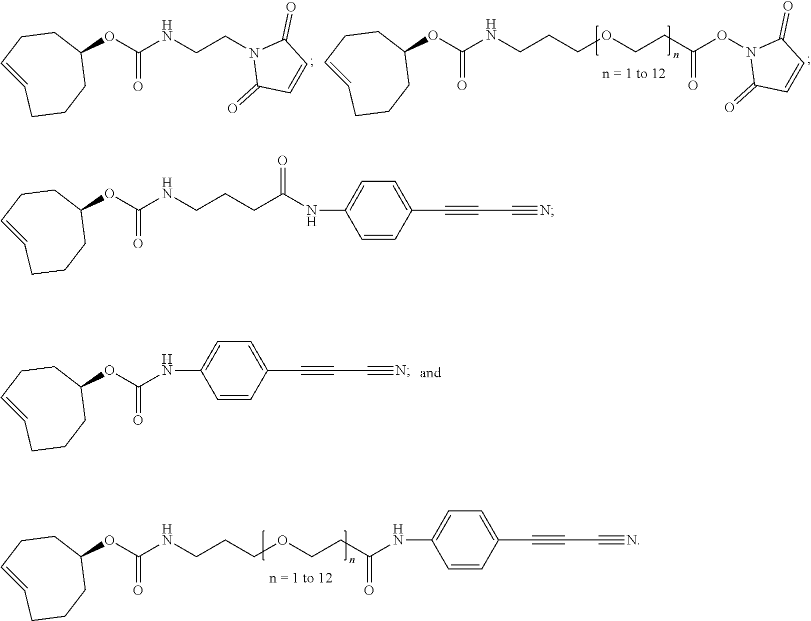

### Embodiment 105, Preparation Method, Limitation of Urate Oxidase Variant Sequence

The method of Embodiment 103 or 104, in which the urate oxidase variant is a tetramer that four urate oxidase variant subunits represented by SEQ ID No: 15 are oligomerized, and X of the SEQ ID No: 15 is frTet.

### Embodiment 106, Preparation Method, Limitation of Albumin

The method of Embodiment 103 or 104, in which the albumin is represented by a sequence selected from among SEQ ID Nos: 46 to 57 or a sequence 90% or more identical to the selected sequence, and the thiol reactive moiety of the linker is bound to the thiol moiety of the 34th cysteine in the albumin sequence through the reaction.

### Embodiment 107, Preparation Method, Limitation of Reaction Environment

The method of any one of Embodiments 103 to 106, in which the reaction between the urate oxidase variant and the linker is performed at a pH of 6 to 8.

### Embodiment 108, Urate Oxidase Variant

A urate oxidase variant containing the following:
a first urate oxidase variant subunit;
a second urate oxidase variant subunit;
a third urate oxidase variant subunit; and
a fourth urate oxidase variant subunit,
in which the urate oxidase variant is a tetramer formed by oligomerization of the first, second, third, and fourth urate oxidase variant subunits,
the first urate oxidase variant subunit is represented by a sequence selected from among SEQ ID Nos: 4 to 17 or a sequence 90% or more identical to the selected sequence,
the second urate oxidase variant subunit is represented by a sequence selected from among SEQ ID Nos: 4 to 17 or a sequence 90% or more identical to the selected sequence,
the third urate oxidase variant subunit is represented by a sequence selected from among SEQ ID Nos: 4 to 17 or a sequence 90% or more identical to the selected sequence, and
the fourth urate oxidase variant subunit is represented by a sequence selected from among SEQ ID Nos: 4 to 17 or a sequence 90% or more identical to the selected sequence,
in which X of the SEQ ID Nos: 4 to 17 is a nonnatural amino acid having a tetrazine functional group or a triazine functional group.

### Embodiment 109, Variant, Limitation of Nonnatural Amino Acid

The variant of Embodiment 108, in which the nonnatural amino acid is selected from among the following:

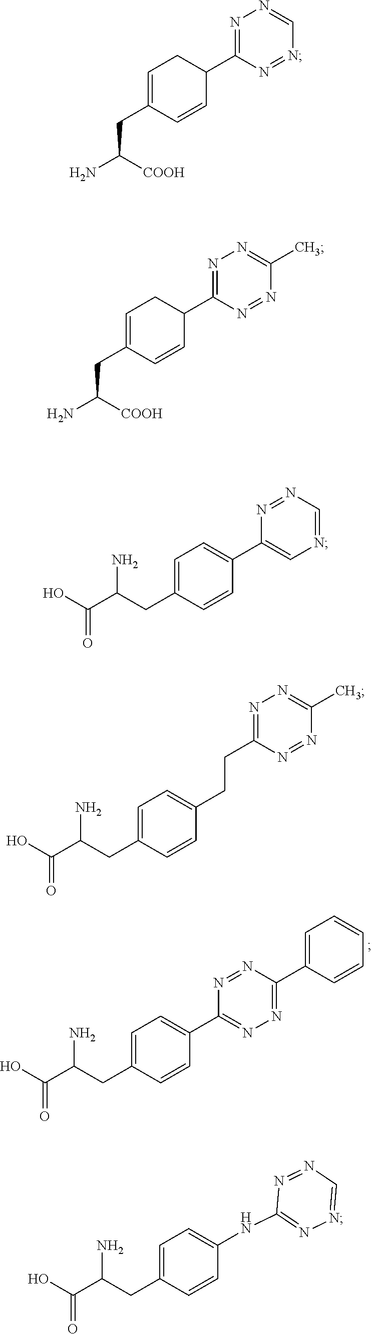 [SMILES: N[C@@H](CC1=CCC(c2nncnn2)C=C1)C(=O)O ; Cc1nnc(C2C=CC(C[C@H](N)C(=O)O)=CC2)nn1 ; NC(Cc1ccc(-c2cncnn2)cc1)C(=O)O ; Cc1nnc(CCc2ccc(CC(N)C(=O)O)cc2)nn1 ; NC(Cc1ccc(-c2nnc(-c3ccccc3)nn2)cc1)C(=O)O ; NC(Cc1ccc(Nc2nncnn2)cc1)C(=O)O]

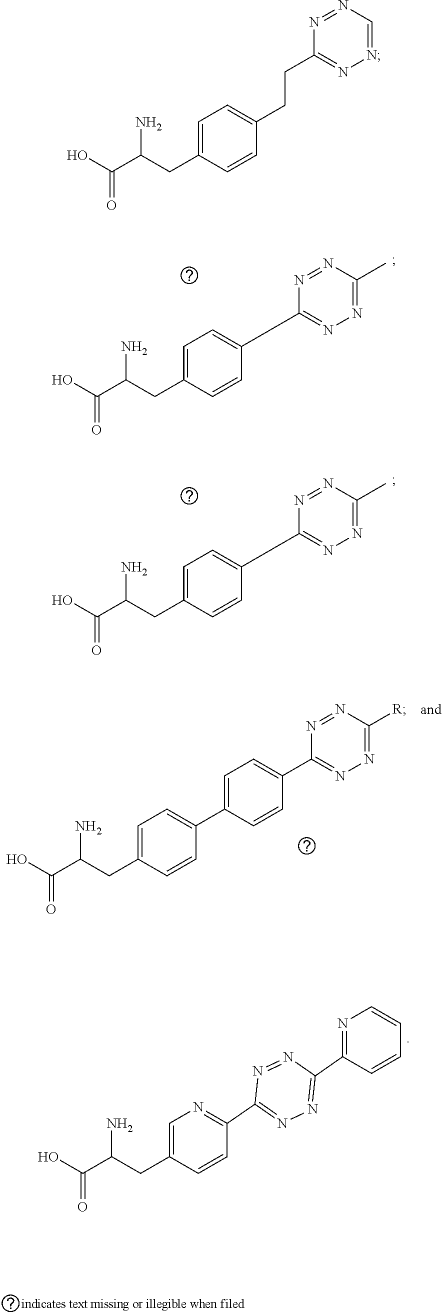

### Embodiment 110, Variant Expression Vector

A urate oxidase variant expression vector encoding a peptide of SEQ ID Nos: 4 to 17,
in which X of the SEQ ID No: 4 to 17 is a nonnatural amino acid, where a vector sequence corresponding to the X is encoded with a stop codon.

### Embodiment 111, Limitation of Stop Codon

The vector of Embodiment 110, in which the stop codon is 5′-UAG-3′.

### Embodiment 112, Variant Preparation Method

A preparation method of a urate oxidase variant, the method including the following:
preparing a vector capable of expressing an orthogonal tRNA/synthetase pair and a cell line containing a urate oxidase variant expression vector,
in which the vector capable of expressing the orthogonal tRNA/synthetase pair is capable of expressing exogenous suppressor tRNA and exogenous tRNA synthetase,
the exogenous suppressor tRNA is capable of recognizing a specific stop codon,
the exogenous tRNA synthetase is capable of recognizing a nonnatural amino acid having a tetrazine functional group and/or triazine functional group so as to be linked to the exogenous suppressor tRNA, and
the urate oxidase variant expression vector is capable of expressing a urate oxidase variant subunit represented by a sequence selected from among SEQ ID Nos: 4 to 17 or a sequence 90% or more identical to the selected sequence, where a site corresponding to the nonnatural amino acid contained in the urate oxidase variant is encoded with the specific stop codon; and
culturing the cell line in a medium containing at least one type of nonnatural amino acid having a tetrazine functional group and/or triazine functional group.

### Embodiment 113, Variant Preparation Method, Limitation of Nonnatural Amino Acid

The method of Embodiment 112, in which the cell line is cultured in the medium containing a nonnatural amino acid selected from among the following:

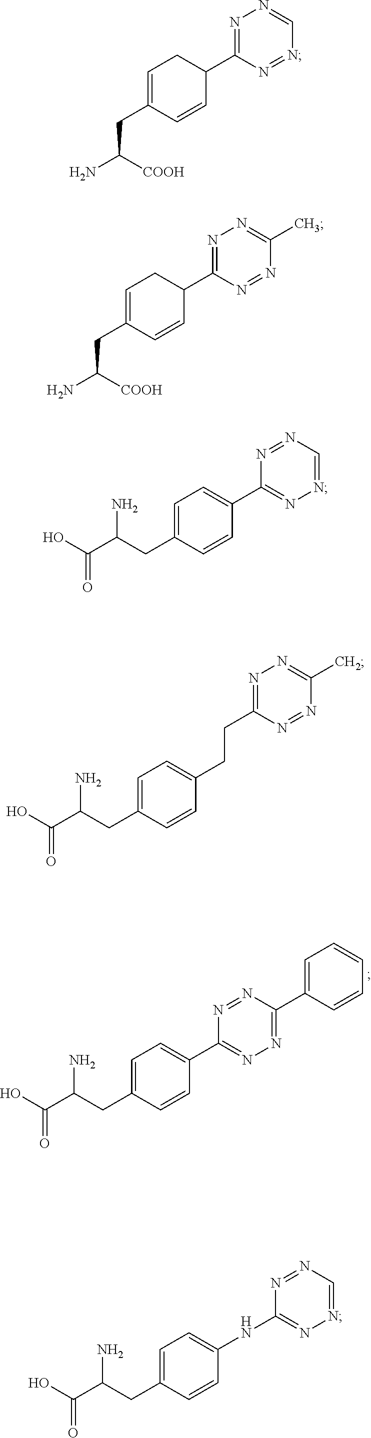

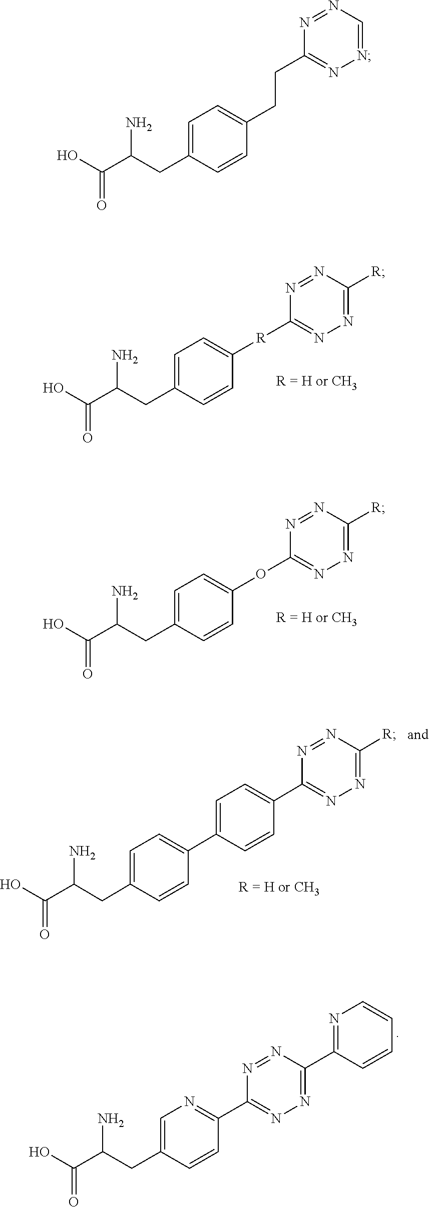

### Embodiment 114, Variant Preparation Method, Limitation of Stop Codon

The method of Embodiment 112 or 113, in which the specific stop codon is 5′-UAG-3′.

### Embodiment 115, Pharmaceutical Composition

A pharmaceutical composition for preventing or treating uric acid-related diseases, the composition containing the following:
the urate oxidase-albumin conjugate of claim 1 in a therapeutically effective dosage; and
a pharmaceutically acceptable carrier.

### Embodiment 116, Pharmaceutical Composition, Limitation of Indication

The composition of Embodiment 115, in which the uric acid-related disease is any one of hyperuricemia, acute gouty arthritis, intermittent gout, chronic nodular gout, chronic kidney disease, and tumor lysis syndrome (TLS).

### Embodiment 117, Pharmaceutical Composition, Limitation of Carrier

The composition of Embodiment 115 or 116, in which the pharmaceutically acceptable carrier contains at least one of the following: binders such as lactose, saccharose, sorbitol, mannitol, starch, amylopectin, cellulose, or gelatin; excipients such as dicalcium phosphate and the like; disintegrants such as corn starch or sweet potato starch; lubricants such as magnesium stearate, calcium stearate, sodium stearyl fumarate, or polyethylene glycol wax; sweetener; air freshener; syrup; liquid carriers such as fatty oils; sterile aqueous solution; propylene glycol; polyethylene glycol; injectable esters such as ethyl oleate; suspending agent; emulsion; freeze-dried preparations; external preparations; stabilizer; buffer; animal oil; vegetable oil; wax; paraffin; starch; tragacanth; cellulose derivatives; polyethylene glycol; silicon; bentonite; silica; talc; and zinc oxide.

### Embodiment 118, Treatment Method

A method of preventing or treating uric acid-related diseases, the method including:
administering the pharmaceutical composition of Embodiment 115 to a subject's body.

### Embodiment 119, Treatment Method, Limitation of Indication

The method of Embodiment 118, in which the uric acid-related disease is any one of hyperuricemia, acute gouty arthritis, intermittent gout, chronic nodular gout, chronic kidney disease, and tumor lysis syndrome (TLS).

### Embodiment 120, Treatment Method, Limitation of Administration Method

The method of Embodiment 118 or 119, in which the administering of the pharmaceutical composition to the subject's body is selected from among oral administration, parenteral administration, intravenous administration, intravenous infusion, intraperitoneal administration, intramuscular administration, transdermal administration, and subcutaneous administration.

### Embodiment 121, Treatment Method, Limitation of Dosage

The method of any one of Embodiments 118 to 120, in which the pharmaceutical composition is administered to the subject's body at a dosage of 0.01 mg/kg to 1000 mg/kg, based on the weight of the administered urate oxidase-albumin conjugate with respect to the weight of the subject.

### MODE FOR INVENTION

### Experimental Example 1 Experiment Materials and Methods

### Experimental Example 1.1 Experiment Materials

4-(1,2,4,5-tetrazin-3-yl) phenylalanine (L-form) used in the disclosed experimental examples was synthesized from WuXi AppTec (China) for use. In addition, recombinant human albumin was purchased from Sartorius Corporation (Bohemia, NY), a Viva spin centrifuge concentrator was purchased from Albumedix (Nottingham, UK), TCO-maleimide (A) was purchased from FutureChem (Seoul, Korea), and Hiprep 26/10 desalting, Superdex 200 increase 10/300 GL and Hitrap IMAC-FF Columns were purchased from Cytiva (Herzogenairach, Germany). TSKgel G3000SWXL HPLC Column was purchased from Tosoh (Tokyo, Japan), and Ni-NTA resin was purchased from Thermo scientific fisher (Waltham, USA). All other chemical reagents were purchased from Sigma-Aldrich Corporation (St. Louis, MO, USA).

### Experimental Example 1.2 Construction of pTAC Plasmid for Protein Expression

To construct a pTAC plasmid, a target protein was codon-optimized. Then, a request was made to Thermo Fisher Scientific for synthesis. The synthesized DNA was amplified by PCR for the target protein with infusion cloning primers. A pTAC-empty vector used as a template plasmid was reacted and linearized under the specified conditions using a KpnI restriction enzyme for preparation. The linearized pTAC-linearized vector and DNA of the target protein amplified by PCR were used according to the Infusion cloning protocol of Takara (Kusatsu, Japan) with the specified primer set under conditions to construct a pTAC-target protein-6H plasmid.

In all PCR processes, PCR products were purified using a Gel/PCR Purification Kit (FAGCK 001, FAVORGEN). The constructed plasmid was transformed into the E. coli DH5alpha strain by a heat shock method. Then, the plasmid was purified by plasmid miniprep and used after sequencing.

### Experimental Example 1.3 Construction of pTAC Plasmid for Protein Variant Expression

Based on the pTAC plasmid constructed in Experimental Example 1.2, a pTAC plasmid for expressing a variant of the target protein was constructed. Specifically, the pTAC-target protein-6H plasmid was infusion-cloned to construct a pTAC plasmid for protein variant expression.

A request was made to Macrogen for preparing a pair of primers for changing all three base sequences responsible for designating the amino acid at the substitution site of the protein variant to be prepared to an amber codon (UAG). Using the pTAC plasmid constructed in Experimental Example 1.2 as a template, codon at each substitution site was substituted with the amber codon by a PCR mixture preparation method using the prepared primer pair.

Then, 1.2 ul of DpnI enzyme was added to a PCR-amplified reaction tube, a reaction was performed at 37° C. for 1 hour to remove the template DNA, and the resulting product was purified using a Gel/PCR Purification Kit (FAGCK 001, FAVORGEN). Next, the specified reaction mixture was prepared to shape the linearized protein variant pTAC plasmid in a circular form.

### Experimental Example 1.4 Preparation and Culture of Protein Variant-Expressing Strain

E. coli C321.Δ.exp (addgene, #49018) strain was inoculated in an LB medium, cultured at 37° C. until OD (600 nm): 0.5˜0.6, spun down, treated with 0.1 M CaCl₂), and then rapidly cooled so as to be prepared into a water-soluble cell form. Then, a pDule-C11RS plasmid was added to E. coli C321.B soluble cells for transformation. E. coli C321.ΔpDule-C11RS] strain was prepared into a water-soluble cell form in the same manner, and the protein variant plasmid constructed in Experimental Example 1.3 was transformed. All transformations were performed by heat shock methods after mixing the water-soluble cells with the plasmid.

The strain for producing the protein variant was inoculated with a colony in a 14-mL culture tube using 3-mL 2×YT (16 g/L Tryptone, 10 g/L Yeast Extract, and 5 g/L NaCl) medium, and cultured under conditions: a temperature of 37° C., a speed of 200 rpm, and a duration of 16 hours, followed by performing the main culture at 1% (v/v) in a 500-mL flask containing a 100-mL 2×YT medium. Antibiotics, kanamycin (35 ug/mL) and tetracycline (10 ug/mL), were added to all 2×YT media used for protein expression, and culture was performed under conditions: a temperature of 37° C. and a speed of 200 rpm until cell growth OD (600 nm): 0.5. After each independently adding 4-(1,2,4,5-tetrazin-3-yl) phenylalanine (L-form) and IPTG ((-D-1-thioglalactopyranoside) at final concentrations of 1 mM, protein overexpression was performed at 25° C. for 16 hours. After the protein overexpression, the cells were collected by centrifugation (13,000×g, 10 min, and 4° C.) and then stored in an ultra-low temperature freezer.

### Experimental Example 1.5 Protein Expression Analysis

The protein variant-expressing cell pellets prepared and cultured according to Experimental Example 1.4 were washed three times using distilled water ten times the volume of the cells, and cell disruption (amp: 30%, 3 sec on, 3 sec off, and 6 min) was performed in a lysis buffer (50 mM sodium phosphate and 300 mM NaCl at pH 8.0) five times the amount of the cells. Next, centrifugation (13,000×g, 30 min, and 4° C.) was performed, and the respective cell disruption supernatants were collected. In the case of the cell disruption pellets, 8 M urea 1 time the amount of the cells was added to release pellet proteins, and protein quantification was performed by the Bradford method, followed by analyzing the same amount of protein by SDS-PAGE. To confirm whether the protein was overexpressed, 1 mM TCO-Cy3 fluorescent linker stock capable of showing fluorescence by incorporating into nonnatural amino acids was used and treated at a final concentration of 20 uM.

### Experimental Example 1.6 Mass Culture of Protein Variants

Among the strains prepared in Experimental Example 1.4, a strain selected for mass production was mass-cultured. Specifically, pre-culture was performed in a 500-mL flask under conditions: a temperature of 37° C., a speed of 220 rpm, and a duration of 16 hours, using 100 mL of 2×YT medium containing kanamycin (35 ug/mL) and tetracycline (10 ug/mL). The main culture was performed in a 1-L flask under conditions: a temperature of 37° C. and a speed of 220 rpm, using 200 mL of the same 2×YT medium. For protein overexpression, when reaching the cell growth OD (600 nm): 0.5, IPTG and nonnatural amino acid 4-(1,2,4,5-tetrazin-3-yl) phenylalanine were added at final concentrations of 1 mM to induce protein overexpression under conditions: a temperature of 25° C., a speed of 220 rpm, and a duration of 16 hours. After completion of the culture, the strain was collected by centrifugation (13,000×g, 10 min, and 4° C.).

### Experimental Example 1.7 Ni-NTA Purification and Analysis

Ni-NTA purification was performed using a wash buffer (50 mM sodium phosphate, 300 mM NaCl, and 20 mM imidazole pH 7.5) and an elution buffer (50 mM sodium phosphate, 300 mM NaCl, and 250 mM imidazole pH 7.5). Each fraction was analyzed by SDS-PAGE using a trans-cyclooctane-Cy3 fluorescent linker.

### Experimental Example 1.8 IMAC-FF Purification

In addition, IMAC-FF FPLC was performed for mass purification. The cell disruption supernatant was filtered using a 0.2-um filter. Using Hitrap IMAC-FF, protein variant purification was performed with an equilibration buffer (50 mM Sodium Phosphate, 300 mM NaCl, 20 mM Imidazole pH 7.5 Buffer) and an elution buffer (50 mM Sodium Phosphate, 300 mM NaCl, 500 mM Imidazole pH 7.5) under the following conditions (equilibration (5 CV equilibration buffer), washing (5 CV, equilibration buffer), 10 CV gradient (0% to 100%), Step 5 CV (100%)).

SDS-PAGE analysis of elution fractions was performed using the trans-cyclooctane-Cy3 fluorescent linker. After the analysis, the protein variant fractions were selected and used as a raw material in the following conjugation step.

### Experimental Example 1.9 SEC-FPLC Purification

The elution fractions were concentrated using Vivaspin (100,000 MWCO) and then purified by SEC-FPLC. SEC-FPLC was performed using a PBS pH 7.4 buffer for equilibration, and through 1.1 CV elution at a flow rate of 0.5 mL/min, each fraction was analyzed by SDS-PAGE.

### Experimental Example 1.10 Preparation of Albumin-Linker Conjugate

To perform conjugation to asparaginase variants using recombinant human albumin (hereinafter referred to as rHA) as a persistent protein, conjugation of TCO-maleimide (A) to rHA was first performed. The TCO-maleimide linker was dissolved with DMSO (dimethyl sulfoxide, sigma) at a concentration of 100 mM for preparation, and the rHA was diluted in a PBS pH 7.4 buffer at a concentration of 150 uM for preparation. Then, a conjugation mixture was prepared at final concentrations of rHA: TCO-maleimide=50 uM: 150 uM. A PBS (pH 7.4) buffer was used as a conjugation buffer, and the conjugation was performed under conditions: a temperature of 23° C., a speed of 130 rpm, and a duration of 3 hours, to prepare an rHA-TCO conjugate.

### Experimental Example 2 Preparation of Asparaginase-Albumin Conjugate

### Experimental Example 2.1 Preparation and Confirmation of Asparaginase and Asparaginase Variant Expression Vector

Asparaginase and asparaginase variant expression vectors were prepared according to Experimental Examples 1.2 to 1.3. In this case, the codon-optimized asparaginase-encoding DNA sequence, asparaginase variant sequence, and primer sequence used are summarized in Tables 1 to 3 below.

TABLE 1

| | | SEQ |
|---|---|---|
| Label | Sequence | ID NO |
| Asparag | ATGGCAGATAAACTGCCGAACATTGTTATTCTGGCAACCGGTGGCACCATTGCAGGTAG | 136 |
| inase WT | CGCAGCAACCGGTACACAGACCACCGGTTATAAAGCCGGTGCACTGGGTGTTGATACCC | |
| | TGATTAATGCAGTTCCGGAAGTTAAAAAGCTGGCCAATGTTAAAGGTGAACAGTTTAGC | |
| | AATATGGCCAGCGAAAATATGACCGGTGATGTTGTTCTGAAACTGAGCCAGCGTGTTAA | |
| | TGAACTGCTGGCACGTGATGATGTTGATGGTGTTGTTATTACCCATGGCACCGATACCG | |
| | TTGAAGAAAGCGCATATTTTCTGCATCTGACCGTGAAAAGCGATAAACCGGTTGTTTTT | |
| | GTTGCAGCAATGCGTCCGGCAACCGCAATTAGCGCAGATGGTCCGATGAATCTGCTGGA | |
| | AGCAGTTCGTGTTGCCGGTGATAAACAGAGCCGTGGTCGTGGTGTTATGGTTGTGCTGA | |
| | ATGATCGTATTGGTAGCGCACGTTATATTACCAAAACCAATGCAAGCACCCTGGATACC | |
| | TTTAAAGCAAATGAAGAAGGTTATCTGGGCGTCATTATTGGCAATCGTATCTATTATCA | |
| | GAACCGCATCGACAAACTGCATACCACACGTAGCGTTTTTGATGTTCGTGGTCTGACCA | |
| | GCCTGCCGAAAGTGGATATTCTGTATGGTTATCAGGATGATCCGGAATATCTGTATGAT | |
| | GCAGCAATTCAGCATGGTGTGAAAGGTATTGTTTATGCAGGTATGGGTGCCGGTAGCGT | |
| | TAGCGTTCGTGGTATTGCCGGTATGCGTAAAGCAATGGAAAAAGGTGTTGTTGTGATTC | |
| | GTAGCACCCGTACCGGTAATGGTATTGTTCCGCCTGATGAAGAACTGCCTGGTCTGGTT | |
| | AGCGATAGCCTGAATCCGGCACATGCACGTATTCTGCTGATGCTGGCACTGACCCGTAC | |
| | CAGCGATCCGAAAGTTATTCAAGAATATTTCCATACCTATTAA | |
| D85UAG | ATGGCAGATAAACTGCCGAACATTGTTATTCTGGCAACCGGTGGCACCATTGCAGGTAG | 100 |
| | CGCAGCAACCGGTACACAGACCACCGGTTATAAAGCCGGTGCACTGGGTGTTGATACCC | |
| | TGATTAATGCAGTTCCGGAAGTTAAAAAGCTGGCCAATGTTAAAGGTGAACAGTTTAGC | |
| | AATATGGCCAGCGAAAATATGACCGGTGATGTTGTTCTGAAACTGAGCCAGCGTGTTAA | |
| | TGAACTGCTGGCACGTTAGGATGTTGATGGTGTTGTTATTACCCATGGCACCGATACCG | |
| | TTGAAGAAAGCGCATATTTTCTGCATCTGACCGTGAAAAGCGATAAACCGGTTGTTTTT | |
| | GTTGCAGCAATGCGTCCGGCAACCGCAATTAGCGCAGATGGTCCGATGAATCTGCTGGA | |
| | AGCAGTTCGTGTTGCCGGTGATAAACAGAGCCGTGGTCGTGGTGTTATGGTTGTGCTGA | |
| | ATGATCGTATTGGTAGCGCACGTTATATTACCAAAACCAATGCAAGCACCCTGGATACC | |
| | TTTAAAGCAAATGAAGAAGGTTATCTGGGCGTCATTATTGGCAATCGTATCTATTATCA | |
| | GAACCGCATCGACAAACTGCATACCACACGTAGCGTTTTTGATGTTCGTGGTCTGACCA | |
| | GCCTGCCGAAAGTGGATATTCTGTATGGTTATCAGGATGATCCGGAATATCTGTATGAT | |
| | GCAGCAATTCAGCATGGTGTGAAAGGTATTGTTTATGCAGGTATGGGTGCCGGTAGCGT | |
| | TAGCGTTCGTGGTATTGCCGGTATGCGTAAAGCAATGGAAAAAGGTGTTGTTGTGATTC | |
| | GTAGCACCCGTACCGGTAATGGTATTGTTCCGCCTGATGAAGAACTGCCTGGTCTGGTT | |
| | AGCGATAGCCTGAATCCGGCACATGCACGTATTCTGCTGATGCTGGCACTGACCCGTAC | |
| | CAGCGATCCGAAAGTTATTCAAGAATATTTCCATACCTATTAA | |
| Q240UAG | ATGGCAGATAAACTGCCGAACATTGTTATTCTGGCAACCGGTGGCACCATTGCAGGTAG | 101 |
| | CGCAGCAACCGGTACACAGACCACCGGTTATAAAGCCGGTGCACTGGGTGTTGATACCC | |
| | TGATTAATGCAGTTCCGGAAGTTAAAAAGCTGGCCAATGTTAAAGGTGAACAGTTTAGC | |
| | AATATGGCCAGCGAAAATATGACCGGTGATGTTGTTCTGAAACTGAGCCAGCGTGTTAA | |
| | TGAACTGCTGGCACGTGATGATGTTGATGGTGTTGTTATTACCCATGGCACCGATACCG | |
| | TTGAAGAAAGCGCATATTTTCTGCATCTGACCGTGAAAAGCGATAAACCGGTTGTTTTT | |
| | GTTGCAGCAATGCGTCCGGCAACCGCAATTAGCGCAGATGGTCCGATGAATCTGCTGGA | |
| | AGCAGTTCGTGTTGCCGGTGATAAACAGAGCCGTGGTCGTGGTGTTATGGTTGTGCTGA | |
| | ATGATCGTATTGGTAGCGCACGTTATATTACCAAAACCAATGCAAGCACCCTGGATACC | |
| | TTTAAAGCAAATGAAGAAGGTTATCTGGGCGTCATTATTGGCAATCGTATCTATTATCA | |
| | GAACCGCATCGACAAACTGCATACCACACGTAGCGTTTTTGATGTTCGTGGTCTGACCA | |
| | GCCTGCCGAAAGTGGATATTCTGTATGGTTATCAGGATGATCCGGAATATCTGTATGAT | |
| | GCAGCAATTTAGCATGGTGTGAAAGGTATTGTTTATGCAGGTATGGGTGCCGGTAGCGT | |
| | TAGCGTTCGTGGTATTGCCGGTATGCGTAAAGCAATGGAAAAAGGTGTTGTTGTGATTC | |
| | GTAGCACCCGTACCGGTAATGGTATTGTTCCGCCTGATGAAGAACTGCCTGGTCTGGTT | |
| | AGCGATAGCCTGAATCCGGCACATGCACGTATTCTGCTGATGCTGGCACTGACCCGTAC | |
| | CAGCGATCCGAAAGTTATTCAAGAATATTTCCATACCTATTAA | |

TABLE 2

| | | SEQ |
|---|---|---|
| Label | Sequence | ID NO |
| E269UAG | ATGGCAGATAAACTGCCGAACATTGTTATTCTGGCAACCGGTGGCACCATTGCAGG | 102 |
| | TAGCGCAGCAACCGGTACACAGACCACCGGTTATAAAGCCGGTGCACTGGGTGTTG | |
| | ATACCCTGATTAATGCAGTTCCGGAAGTTAAAAAGCTGGCCAATGTTAAAGGTGAA | |
| | CAGTTTAGCAATATGGCCAGCGAAAATATGACCGGTGATGTTGTTCTGAAACTGAG | |
| | CCAGCGTGTTAATGAACTGCTGGCACGTGATGATGTTGATGGTGTTGTTATTACCC | |
| | ATGGCACCGATACCGTTGAAGAAAGCGCATATTTTCTGCATCTGACCGTGAAAAGC | |
| | GATAAACCGGTTGTTTTTGTTGCAGCAATGCGTCCGGCAACCGCAATTAGCGCAGA | |
| | TGGTCCGATGAATCTGCTGGAAGCAGTTCGTGTTGCCGGTGATAAACAGAGCCGTG | |
| | GTCGTGGTGTTATGGTTGTGCTGAATGATCGTATTGGTAGCGCACGTTATATTACC | |
| | AAAACCAATGCAAGCACCCTGGATACCTTTAAAGCAAATGAAGAAGGTTATCTGGG | |
| | CGTCATTATTGGCAATCGTATCTATTATCAGAACCGCATCGACAAACTGCATACCA | |
| | CACGTAGCGTTTTTGATGTTCGTGGTCTGACCAGCCTGCCGAAAGTGGATATTCTG | |
| | TATGGTTATCAGGATGATCCGGAATATCTGTATGATGCAGCAATTCAGCATGGTGT | |
| | GAAAGGTATTGTTTATGCAGGTATGGGTGCCGGTAGCGTTAGCGTTCGTGGTATTG | |
| | CCGGTATGCGTAAAGCAATGTAGAAAGGTGTTGTTGTGATTCGTAGCACCCGTACC | |
| | GGTAATGGTATTGTTCCGCCTGATGAAGAACTGCCTGGTCTGGTTAGCGATAGCCT | |
| | GAATCCGGCACATGCACGTATTCTGCTGATGCTGGCACTGACCCGTACCAGCGATC | |
| | CGAAAGTTATTCAAGAATATTTCCATACCTATTAA | |
| E289UAG | ATGGCAGATAAACTGCCGAACATTGTTATTCTGGCAACCGGTGGCACCATTGCAGG | 103 |
| | TAGCGCAGCAACCGGTACACAGACCACCGGTTATAAAGCCGGTGCACTGGGTGTTG | |
| | ATACCCTGATTAATGCAGTTCCGGAAGTTAAAAAGCTGGCCAATGTTAAAGGTGAA | |
| | CAGTTTAGCAATATGGCCAGCGAAAATATGACCGGTGATGTTGTTCTGAAACTGAG | |
| | CCAGCGTGTTAATGAACTGCTGGCACGTGATGATGTTGATGGTGTTGTTATTACCC | |
| | ATGGCACCGATACCGTTGAAGAAAGCGCATATTTTCTGCATCTGACCGTGAAAAGC | |
| | GATAAACCGGTTGTTTTTGTTGCAGCAATGCGTCCGGCAACCGCAATTAGCGCAGA | |
| | TGGTCCGATGAATCTGCTGGAAGCAGTTCGTGTTGCCGGTGATAAACAGAGCCGTG | |
| | GTCGTGGTGTTATGGTTGTGCTGAATGATCGTATTGGTAGCGCACGTTATATTACC | |
| | AAAACCAATGCAAGCACCCTGGATACCTTTAAAGCAAATGAAGAAGGTTATCTGGG | |
| | CGTCATTATTGGCAATCGTATCTATTATCAGAACCGCATCGACAAACTGCATACCA | |
| | CACGTAGCGTTTTTGATGTTCGTGGTCTGACCAGCCTGCCGAAAGTGGATATTCTG | |
| | TATGGTTATCAGGATGATCCGGAATATCTGTATGATGCAGCAATTCAGCATGGTGT | |
| | GAAAGGTATTGTTTATGCAGGTATGGGTGCCGGTAGCGTTAGCGTTCGTGGTATTG | |
| | CCGGTATGCGTAAAGCAATGGAAAAAGGTGTTGTTGTGATTCGTAGCACCCGTACC | |
| | GGTAATGGTATTGTTCCGCCTTAGGAAGAACTGCCTGGTCTGGTTAGCGATAGCCT | |
| | GAATCCGGCACATGCACGTATTCTGCTGATGCTGGCACTGACCCGTACCAGCGATC | |
| | CGAAAGTTATTCAAGAATATTTCCATACCTATTAA | |
| K319UAG | ATGGCAGATAAACTGCCGAACATTGTTATTCTGGCAACCGGTGGCACCATTGCAGG | 104 |
| | TAGCGCAGCAACCGGTACACAGACCACCGGTTATAAAGCCGGTGCACTGGGTGTTG | |
| | ATACCCTGATTAATGCAGTTCCGGAAGTTAAAAAGCTGGCCAATGTTAAAGGTGAA | |
| | CAGTTTAGCAATATGGCCAGCGAAAATATGACCGGTGATGTTGTTCTGAAACTGAG | |
| | CCAGCGTGTTAATGAACTGCTGGCACGTGATGATGTTGATGGTGTTGTTATTACCC | |
| | ATGGCACCGATACCGTTGAAGAAAGCGCATATTTTCTGCATCTGACCGTGAAAAGC | |
| | GATAAACCGGTTGTTTTTGTTGCAGCAATGCGTCCGGCAACCGCAATTAGCGCAGA | |
| | TGGTCCGATGAATCTGCTGGAAGCAGTTCGTGTTGCCGGTGATAAACAGAGCCGTG | |
| | GTCGTGGTGTTATGGTTGTGCTGAATGATCGTATTGGTAGCGCACGTTATATTACC | |
| | AAAACCAATGCAAGCACCCTGGATACCTTTAAAGCAAATGAAGAAGGTTATCTGGG | |
| | CGTCATTATTGGCAATCGTATCTATTATCAGAACCGCATCGACAAACTGCATACCA | |
| | CACGTAGCGTTTTTGATGTTCGTGGTCTGACCAGCCTGCCGAAAGTGGATATTCTG | |
| | TATGGTTATCAGGATGATCCGGAATATCTGTATGATGCAGCAATTCAGCATGGTGT | |
| | GAAAGGTATTGTTTATGCAGGTATGGGTGCCGGTAGCGTTAGCGTTCGTGGTATTG | |
| | CCGGTATGCGTAAAGCAATGGAAAAAGGTGTTGTTGTGATTCGTAGCACCCGTACC | |
| | GGTAATGGTATTGTTCCGCCTGATGAAGAACTGCCTGGTCTGGTTAGCGATAGCCT | |
| | GAATCCGGCACATGCACGTATTCTGCTGATGCTGGCACTGACCCGTACCAGCGATC | |
| | CGTAGGTTATTCAAGAATATTTCCATACCTATTAA | |

TABLE 3

| | | SEQ ID |
|---|---|---|
| Label | Sequence | NO |
| pTAC- | AGATATAGGGATCCGGTACCATGGCAGATAAACTGCCGAACA | 105 |
| Asparaginase- | | |
| Infusion-F | | |
| pTAC- | CTATCATCACCATCACCATCACTAAGGTACCAAGCTTGGCTGTTT | 106 |
| Asparaginase- | | |
| Infusion-R | | |
| Asparaginase- | GGCACGTTAGGATGTTGATGGTGTTGTTATTACCC | 107 |
| D85UAG-F | | |
| Asparaginase- | ACATCCTAACGTGCCAGCAGTTCATTAACAC | 108 |
| D85UAG-R | | |
| Asparaginase- | AGCAATGTAGAAAGGTGTTGTTGTGATTCGTAGCA | 109 |
| E269UAG-F | | |
| Asparaginase- | CCTTTCTACATTGCTTTACGCATACCGGCA | 110 |
| E269UAG-R | | |
| Asparaginase- | CGATCCGTAGGTTATTCAAGAATATTTCCATACCT | 111 |
| K319UAG-F | | |
| Asparaginase- | ATAACCTACGGATCGCTGGTACGGGT | 112 |
| K319UAG-R | | |
| Asparaginase- | AGCAATTTAGCATGGTGTGAAAGGTATTGTTTATG | 113 |
| Q240UAG-F | | |
| Asparaginase- | CCATGCTAAATTGCTGCATCATACAGATATTCC | 114 |
| Q240UAG-R | | |
| Asparaginase- | GCCTGATTAGGAACTGCCTGGTCTGGTTAGC | 115 |
| E289UAG-F | | |
| Asparaginase- | AGTTCCTAATCAGGCGGAACAATACCATTACC | 116 |
| E289UAG-R | | |

The five types of constructed pTAC-Asparaginase-6H variants plasmid were each independently transformed into E. coli DH5alpha strain and purified by plasmid miniprep. Then, the plasmids were digested with a KpnI restriction enzyme, and a 1011-bp insert DNA band was confirmed through electrophoresis in a 1% agarose medium.

### Experimental Example 2.2 Selection of Asparaginase Variant

Using the asparaginase and the variant expression vector thereof constructed according to Experimental Example 2.1, the asparaginase and the variants thereof were expressed according to

### Experimental Examples 1.4 to 1.5 and Analyzed

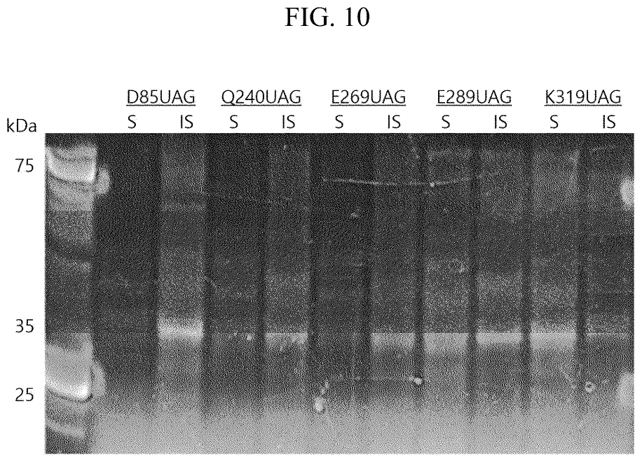

As a result of SDS-PAGE, it was confirmed that the asparaginase variant in which a nonnatural amino acid was introduced into the amino acid lysine at position 319 exhibited the highest soluble protein expression amount and was thus selected as a target for mass production (FIG. 10).

### Experimental Example 2.3 Obtainment of Asparaginase Variant

To obtain the asparaginase variant selected in Experimental Example 2.2, the corresponding strain was mass-cultured according to Experimental Example 1.6, and the cells were disrupted through the following process.

The wet cells of the mass-cultured strain in Experimental Example 1.6 were disrupted using an ultrasonicator. The strain was washed three times with distilled water ten times the volume with respect to the weight of the cells, and as a lysis buffer, 50 mM sodium phosphate, 300 mM NaCl, and 20 mM imidazole pH 7.5 buffer were used. Ultrasonic disruption was performed for 6 minutes under the conditions of amplification factor: 30%, 3 sec on, 3 sec off. After the disruption, the supernatant was collected by centrifugation (13,000×g, 30 min, and 4° C.). Affinity chromatography was performed on a portion of the cell disruption supernatant using Ni-NTA resin to confirm the binding affinity of a 6× histidine tag.

Then, purification and analysis were performed according to Experimental Examples 1.7 to 1.9.

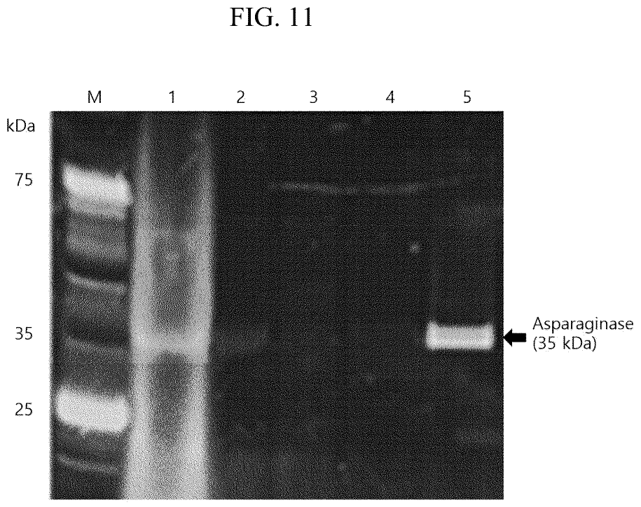

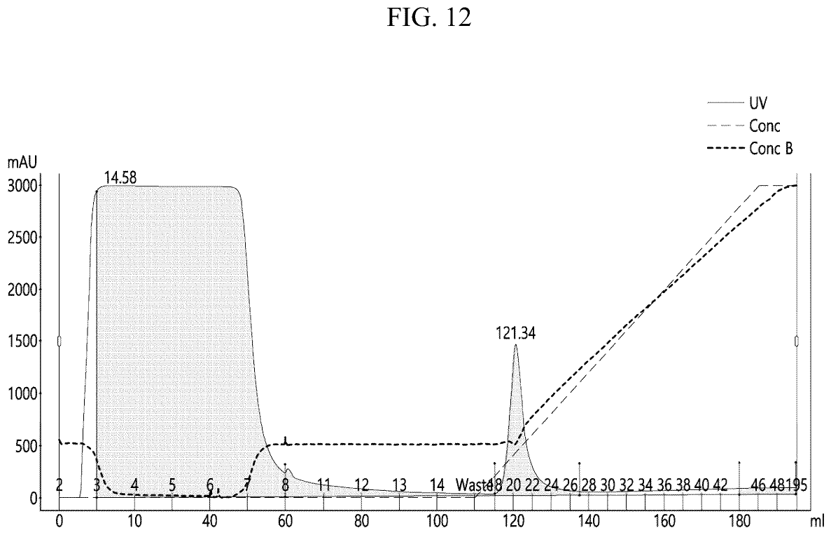

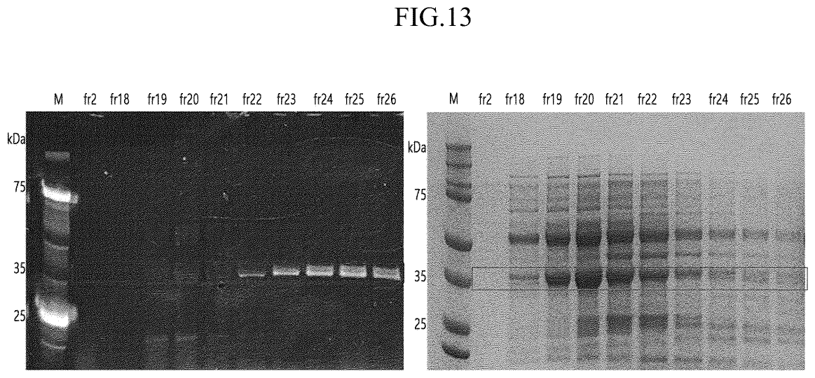

As a result of the analysis, it was confirmed that the asparaginase (K319UAG)-6× histidine-tagged protein was normally eluted in the elution fractions (FIG. 11). In addition, results of performing IMAC-FF FPLC for mass purification are shown in FIGS. 12 to 13.

### Experimental Example 2.4 Preparation of Asparaginase-Albumin Conjugate

An asparaginase-albumin conjugate was prepared by using the albumin-linker conjugate prepared according to Experimental Example 1.10 with the asparaginase variant obtained in Experimental Example 2.3. Specifically, during the IMAC purification process, not only the protein variants but also other proteins were eluted, so the yield per 1 g wet cell was set to 0.625 mg/g wet cell. In addition, using 1.25 mg rHA-TCO per 1 g wet cell, protein variant-rHA conjugation was performed based on rHA at a concentration of 50 uM. The conjugation was performed under conditions: a temperature of 23° C., a speed of 130 rpm, and a duration of 16 hours.

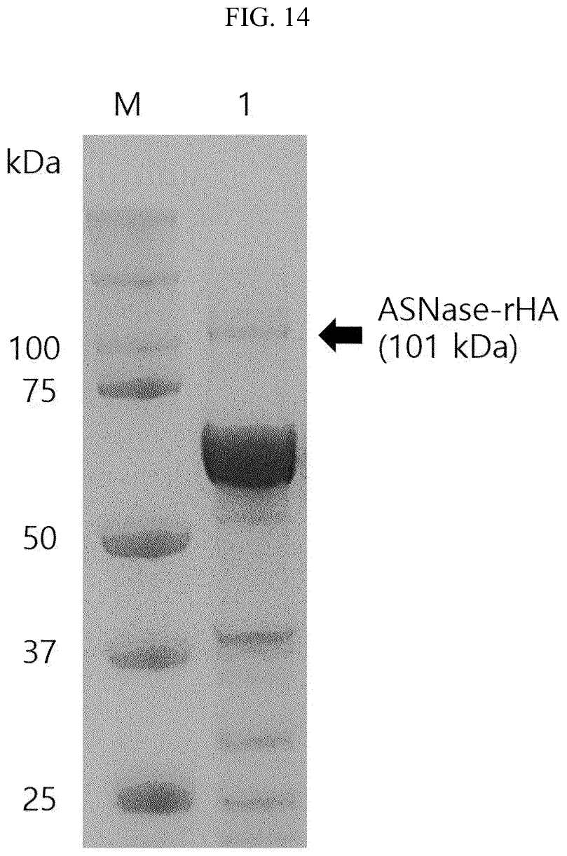

As a result of SDS-PAGE analysis, a 101 kDa protein band of the asparaginase-albumin conjugate was confirmed (FIG. 14).

### Experimental Example 2.5 Evaluation of Asparaginase-Albumin Conjugate Activity

Evaluation of asparaginase activity was performed using an asparaginase assay kit purchased from Sigma while using E. coli-derived asparaginase as a control, and the concentration of ammonia was quantified with Nessler's reagent to determine unit activity.

### Experimental Example 3 Preparation and Evaluation of Methioninase-Albumin Conjugate

### Experimental Example 3.1 Preparation of Methioninase and Methioninase Variant Expression Vectors

Methioninase and methioninase variant expression vectors were prepared according to Experimental Examples 1.2 to 1.3. In this case, the codon-optimized methioninase-encoding DNA sequence, methioninase variant sequence, and primer sequence used are summarized in Tables 4 to 6 below.

TABLE 4

| | | SEQ ID |
|---|---|---|
| Label | Sequence | NO |
| Methioninase | ATGCATGGCAGCAATAAACTGCCTGGTTTTGCAACCCGTGCAATTCATCATGGTTA | 137 |
| WT | TGATCCACAGGATCATGGTGGTGCACTGGTTCCGCCTGTTTATCAGACCGCAACCT | |
| | TTACCTTTCCGACCGTTGAATATGGTGCAGCATGTTTTGCCGGTGAACAGGCAGGT | |
| | CACTTTTATAGCCGTATTAGCAATCCGACACTGAATCTGCTGGAAGCACGTATGGC | |
| | AAGCCTGGAAGGTGGTGAAGCAGGTCTGGCACTGGCAAGCGGTATGGGTGCAATTA | |
| | CCAGCACACTGTGGACCCTGCTGCGTCCGGGTGATGAAGTTCTGCTGGGTAATACC | |
| | CTGTATGGTTGTACCTTTGCATTTCTGCATCATGGCATTGGTGAATTTGGTGTTAA | |
| | ACTGCGTCATGTTGATATGGCCGATCTGCAGGCACTGGAAGCAGCAATGACACCGG | |
| | CAACACGTGTGATTTATTTCGAAAGTCCGGCAAATCCGAACATGCATATGGCAGAT | |
| | ATTGCGGGTGTTGCAAAAATTGCACGTAAACATGGTGCAACCGTTGTTGTGGATAA | |
| | TACCTATTGTACCCCGTATCTGCAGCGTCCGCTGGAACTGGGTGCAGATCTGGTTG | |
| | TTCATAGCGCAACCAAATATCTGAGCGGTCATGGTGATATTACCGCAGGTATTGTT | |
| | GTTGGTAGCCAGGCGCTGGTTGATCGTATTCGTCTGCAGGGTCTGAAAGATATGAC | |
| | CGGTGCAGTTCTGAGTCCGCATGATGCCGCACTGCTGATGCGTGGTATTAAAACCC | |
| | TGAACCTGCGTATGGATCGTCATTGTGCAAATGCACAGGTTCTGGCAGAATTTCTG | |
| | GCACGTCAGCCGCAGGTTGAACTGATTCATTATCCTGGTCTGGCAAGCTTTCCGCA | |
| | GTATACCCTGGCACGCCAGCAGATGAGCCAGCCTGGTGGTATGATTGCATTTGAAC | |
| | TGAAAGGTGGTATTGGTGCAGGTCGTCGTTTTATGAATGCACTGCAGCTGTTTAGC | |
| | CGTGCAGTTAGCCTGGGTGATGCCGAAAGCCTGGCACAGCATCCGGCAAGCATGAC | |
| | CCATAGCAGCTATACACCGGAAGAACGTGCACATTATGGTATTAGCGAAGGTCTGG | |
| | TTCGTCTGAGCGTTGGCCTGGAAGATATTGATGACCTGCTGGCAGATGTTCAGCAG | |
| | GCCCTGAAAGCAAGCGCATAA | |
| A144UAG | ATGCATGGCAGCAATAAACTGCCTGGTTTTGCAACCCGTGCAATTCATCATGGTTA | 117 |
| | TGATCCACAGGATCATGGTGGTGCACTGGTTCCGCCTGTTTATCAGACCGCAACCT | |
| | TTACCTTTCCGACCGTTGAATATGGTGCAGCATGTTTTGCCGGTGAACAGGCAGGT | |
| | CACTTTTATAGCCGTATTAGCAATCCGACACTGAATCTGCTGGAAGCACGTATGGC | |
| | AAGCCTGGAAGGTGGTGAAGCAGGTCTGGCACTGGCAAGCGGTATGGGTGCAATTA | |
| | CCAGCACACTGTGGACCCTGCTGCGTCCGGGTGATGAAGTTCTGCTGGGTAATACC | |
| | CTGTATGGTTGTACCTTTGCATTTCTGCATCATGGCATTGGTGAATTTGGTGTTAA | |
| | ACTGCGTCATGTTGATATGGCCGATCTGCAGGCACTGGAATAGGCAATGACACCGG | |
| | CAACACGTGTGATTTATTTCGAAAGTCCGGCAAATCCGAACATGCATATGGCAGAT | |
| | ATTGCGGGTGTTGCAAAAATTGCACGTAAACATGGTGCAACCGTTGTTGTGGATAA | |
| | TACCTATTGTACCCCGTATCTGCAGCGTCCGCTGGAACTGGGTGCAGATCTGGTTG | |
| | TTCATAGCGCAACCAAATATCTGAGCGGTCATGGTGATATTACCGCAGGTATTGTT | |
| | GTTGGTAGCCAGGCGCTGGTTGATCGTATTCGTCTGCAGGGTCTGAAAGATATGAC | |
| | CGGTGCAGTTCTGAGTCCGCATGATGCCGCACTGCTGATGCGTGGTATTAAAACCC | |
| | TGAACCTGCGTATGGATCGTCATTGTGCAAATGCACAGGTTCTGGCAGAATTTCTG | |
| | GCACGTCAGCCGCAGGTTGAACTGATTCATTATCCTGGTCTGGCAAGCTTTCCGCA | |
| | GTATACCCTGGCACGCCAGCAGATGAGCCAGCCTGGTGGTATGATTGCATTTGAAC | |
| | TGAAAGGTGGTATTGGTGCAGGTCGTCGTTTTATGAATGCACTGCAGCTGTTTAGC | |
| | CGTGCAGTTAGCCTGGGTGATGCCGAAAGCCTGGCACAGCATCCGGCAAGCATGAC | |
| | CCATAGCAGCTATACACCGGAAGAACGTGCACATTATGGTATTAGCGAAGGTCTGG | |
| | TTCGTCTGAGCGTTGGCCTGGAAGATATTGATGACCTGCTGGCAGATGTTCAGCAG | |
| | GCCCTGAAAGCAAGCGCATAA | |
| P148UAG | ATGCATGGCAGCAATAAACTGCCTGGTTTTGCAACCCGTGCAATTCATCATGGTTA | 118 |
| | TGATCCACAGGATCATGGTGGTGCACTGGTTCCGCCTGTTTATCAGACCGCAACCT | |
| | TTACCTTTCCGACCGTTGAATATGGTGCAGCATGTTTTGCCGGTGAACAGGCAGGT | |
| | CACTTTTATAGCCGTATTAGCAATCCGACACTGAATCTGCTGGAAGCACGTATGGC | |
| | AAGCCTGGAAGGTGGTGAAGCAGGTCTGGCACTGGCAAGCGGTATGGGTGCAATTA | |
| | CCAGCACACTGTGGACCCTGCTGCGTCCGGGTGATGAAGTTCTGCTGGGTAATACC | |
| | CTGTATGGTTGTACCTTTGCATTTCTGCATCATGGCATTGGTGAATTTGGTGTTAA | |
| | ACTGCGTCATGTTGATATGGCCGATCTGCAGGCACTGGAAGCAGCAATGACATAGG | |
| | CAACACGTGTGATTTATTTCGAAAGTCCGGCAAATCCGAACATGCATATGGCAGAT | |
| | ATTGCGGGTGTTGCAAAAATTGCACGTAAACATGGTGCAACCGTTGTTGTGGATAA | |
| | TACCTATTGTACCCCGTATCTGCAGCGTCCGCTGGAACTGGGTGCAGATCTGGTTG | |
| | TTCATAGCGCAACCAAATATCTGAGCGGTCATGGTGATATTACCGCAGGTATTGTT | |
| | GTTGGTAGCCAGGCGCTGGTTGATCGTATTCGTCTGCAGGGTCTGAAAGATATGAC | |
| | CGGTGCAGTTCTGAGTCCGCATGATGCCGCACTGCTGATGCGTGGTATTAAAACCC | |
| | TGAACCTGCGTATGGATCGTCATTGTGCAAATGCACAGGTTCTGGCAGAATTTCTG | |
| | GCACGTCAGCCGCAGGTTGAACTGATTCATTATCCTGGTCTGGCAAGCTTTCCGCA | |
| | GTATACCCTGGCACGCCAGCAGATGAGCCAGCCTGGTGGTATGATTGCATTTGAAC | |
| | TGAAAGGTGGTATTGGTGCAGGTCGTCGTTTTATGAATGCACTGCAGCTGTTTAGC | |
| | CGTGCAGTTAGCCTGGGTGATGCCGAAAGCCTGGCACAGCATCCGGCAAGCATGAC | |
| | CCATAGCAGCTATACACCGGAAGAACGTGCACATTATGGTATTAGCGAAGGTCTGG | |
| | TTCGTCTGAGCGTTGGCCTGGAAGATATTGATGACCTGCTGGCAGATGTTCAGCAG | |
| | GCCCTGAAAGCAAGCGCATAA | |

TABLE 5

| | | SEQ |
|---|---|---|
| Label | Sequence | ID NO |
| K177UAG | ATGCATGGCAGCAATAAACTGCCTGGTTTTGCAACCCGTGCAATTCATCATGGTTA | 119 |
| | TGATCCACAGGATCATGGTGGTGCACTGGTTCCGCCTGTTTATCAGACCGCAACCT | |
| | TTACCTTTCCGACCGTTGAATATGGTGCAGCATGTTTTGCCGGTGAACAGGCAGGT | |
| | CACTTTTATAGCCGTATTAGCAATCCGACACTGAATCTGCTGGAAGCACGTATGGC | |
| | AAGCCTGGAAGGTGGTGAAGCAGGTCTGGCACTGGCAAGCGGTATGGGTGCAATTA | |
| | CCAGCACACTGTGGACCCTGCTGCGTCCGGGTGATGAAGTTCTGCTGGGTAATACC | |
| | CTGTATGGTTGTACCTTTGCATTTCTGCATCATGGCATTGGTGAATTTGGTGTTAA | |
| | ACTGCGTCATGTTGATATGGCCGATCTGCAGGCACTGGAAGCAGCAATGACACCGG | |
| | CAACACGTGTGATTTATTTCGAAAGTCCGGCAAATCCGAACATGCATATGGCAGAT | |
| | ATTGCGGGTGTTGCAAAAATTGCACGTTAGCATGGTGCAACCGTTGTTGTGGATAA | |
| | TACCTATTGTACCCCGTATCTGCAGCGTCCGCTGGAACTGGGTGCAGATCTGGTTG | |
| | TTCATAGCGCAACCAAATATCTGAGCGGTCATGGTGATATTACCGCAGGTATTGTT | |
| | GTTGGTAGCCAGGCGCTGGTTGATCGTATTCGTCTGCAGGGTCTGAAAGATATGAC | |
| | CGGTGCAGTTCTGAGTCCGCATGATGCCGCACTGCTGATGCGTGGTATTAAAACCC | |
| | TGAACCTGCGTATGGATCGTCATTGTGCAAATGCACAGGTTCTGGCAGAATTTCTG | |
| | GCACGTCAGCCGCAGGTTGAACTGATTCATTATCCTGGTCTGGCAAGCTTTCCGCA | |
| | GTATACCCTGGCACGCCAGCAGATGAGCCAGCCTGGTGGTATGATTGCATTTGAAC | |
| | TGAAAGGTGGTATTGGTGCAGGTCGTCGTTTTATGAATGCACTGCAGCTGTTTAGC | |
| | CGTGCAGTTAGCCTGGGTGATGCCGAAAGCCTGGCACAGCATCCGGCAAGCATGAC | |
| | CCATAGCAGCTATACACCGGAAGAACGTGCACATTATGGTATTAGCGAAGGTCTGG | |
| | TTCGTCTGAGCGTTGGCCTGGAAGATATTGATGACCTGCTGGCAGATGTTCAGCAG | |
| | GCCCTGAAAGCAAGCGCATAA | |
| P283UAG | ATGCATGGCAGCAATAAACTGCCTGGTTTTGCAACCCGTGCAATTCATCATGGTTA | 120 |
| | TGATCCACAGGATCATGGTGGTGCACTGGTTCCGCCTGTTTATCAGACCGCAACCT | |
| | TTACCTTTCCGACCGTTGAATATGGTGCAGCATGTTTTGCCGGTGAACAGGCAGGT | |
| | CACTTTTATAGCCGTATTAGCAATCCGACACTGAATCTGCTGGAAGCACGTATGGC | |
| | AAGCCTGGAAGGTGGTGAAGCAGGTCTGGCACTGGCAAGCGGTATGGGTGCAATTA | |
| | CCAGCACACTGTGGACCCTGCTGCGTCCGGGTGATGAAGTTCTGCTGGGTAATACC | |
| | CTGTATGGTTGTACCTTTGCATTTCTGCATCATGGCATTGGTGAATTTGGTGTTAA | |
| | ACTGCGTCATGTTGATATGGCCGATCTGCAGGCACTGGAAGCAGCAATGACACCGG | |
| | CAACACGTGTGATTTATTTCGAAAGTCCGGCAAATCCGAACATGCATATGGCAGAT | |
| | ATTGCGGGTGTTGCAAAAATTGCACGTAAACATGGTGCAACCGTTGTTGTGGATAA | |
| | TACCTATTGTACCCCGTATCTGCAGCGTCCGCTGGAACTGGGTGCAGATCTGGTTG | |
| | TTCATAGCGCAACCAAATATCTGAGCGGTCATGGTGATATTACCGCAGGTATTGTT | |
| | GTTGGTAGCCAGGCGCTGGTTGATCGTATTCGTCTGCAGGGTCTGAAAGATATGAC | |
| | CGGTGCAGTTCTGAGTCCGCATGATGCCGCACTGCTGATGCGTGGTATTAAAACCC | |
| | TGAACCTGCGTATGGATCGTCATTGTGCAAATGCACAGGTTCTGGCAGAATTTCTG | |
| | GCACGTCAGTAGCAGGTTGAACTGATTCATTATCCTGGTCTGGCAAGCTTTCCGCA | |
| | GTATACCCTGGCACGCCAGCAGATGAGCCAGCCTGGTGGTATGATTGCATTTGAAC | |
| | TGAAAGGTGGTATTGGTGCAGGTCGTCGTTTTATGAATGCACTGCAGCTGTTTAGC | |
| | CGTGCAGTTAGCCTGGGTGATGCCGAAAGCCTGGCACAGCATCCGGCAAGCATGAC | |
| | CCATAGCAGCTATACACCGGAAGAACGTGCACATTATGGTATTAGCGAAGGTCTGG | |
| | TTCGTCTGAGCGTTGGCCTGGAAGATATTGATGACCTGCTGGCAGATGTTCAGCAG | |
| | GCCCTGAAAGCAAGCGCATAA | |
| T300UAG | ATGCATGGCAGCAATAAACTGCCTGGTTTTGCAACCCGTGCAATTCATCATGGTTA | 121 |
| | TGATCCACAGGATCATGGTGGTGCACTGGTTCCGCCTGTTTATCAGACCGCAACCT | |
| | TTACCTTTCCGACCGTTGAATATGGTGCAGCATGTTTTGCCGGTGAACAGGCAGGT | |
| | CACTTTTATAGCCGTATTAGCAATCCGACACTGAATCTGCTGGAAGCACGTATGGC | |
| | AAGCCTGGAAGGTGGTGAAGCAGGTCTGGCACTGGCAAGCGGTATGGGTGCAATTA | |
| | CCAGCACACTGTGGACCCTGCTGCGTCCGGGTGATGAAGTTCTGCTGGGTAATACC | |
| | CTGTATGGTTGTACCTTTGCATTTCTGCATCATGGCATTGGTGAATTTGGTGTTAA | |
| | ACTGCGTCATGTTGATATGGCCGATCTGCAGGCACTGGAAGCAGCAATGACACCGG | |
| | CAACACGTGTGATTTATTTCGAAAGTCCGGCAAATCCGAACATGCATATGGCAGAT | |
| | ATTGCGGGTGTTGCAAAAATTGCACGTAAACATGGTGCAACCGTTGTTGTGGATAA | |
| | TACCTATTGTACCCCGTATCTGCAGCGTCCGCTGGAACTGGGTGCAGATCTGGTTG | |
| | TTCATAGCGCAACCAAATATCTGAGCGGTCATGGTGATATTACCGCAGGTATTGTT | |
| | GTTGGTAGCCAGGCGCTGGTTGATCGTATTCGTCTGCAGGGTCTGAAAGATATGAC | |
| | CGGTGCAGTTCTGAGTCCGCATGATGCCGCACTGCTGATGCGTGGTATTAAAACCC | |
| | TGAACCTGCGTATGGATCGTCATTGTGCAAATGCACAGGTTCTGGCAGAATTTCTG | |
| | GCACGTCAGCCGCAGGTTGAACTGATTCATTATCCTGGTCTGGCAAGCTTTCCGCA | |
| | GTATTAGCTGGCACGCCAGCAGATGAGCCAGCCTGGTGGTATGATTGCATTTGAAC | |
| | TGAAAGGTGGTATTGGTGCAGGTCGTCGTTTTATGAATGCACTGCAGCTGTTTAGC | |
| | CGTGCAGTTAGCCTGGGTGATGCCGAAAGCCTGGCACAGCATCCGGCAAGCATGAC | |
| | CCATAGCAGCTATACACCGGAAGAACGTGCACATTATGGTATTAGCGAAGGTCTGG | |
| | TTCGTCTGAGCGTTGGCCTGGAAGATATTGATGACCTGCTGGCAGATGTTCAGCAG | |
| | GCCCTGAAAGCAAGCGCATAA | |

TABLE 6

| | | SEQ ID |
|---|---|---|
| Label | Sequence | NO |
| pTAC-6h-Methioninase- | AGATATAGGGATCCGGTACCATGGCAGATAAACTGCCGAACA | 122 |
| Infusion-F | | |
| pTAC-6h-Methioninase- | CTATCATCACCATCACCATCACTAAGGTACCAAGCTTGGCTG | 123 |
| Infusion-R | TTT | |
| 6h-Methioninase- | GGCACGTTAGGATGTTGATGGTGTTGTTATTACCC | 124 |
| D85UAG-F | | |
| 6h-Methioninase- | ACATCCTAACGTGCCAGCAGTTCATTAACAC | 125 |
| D85UAG-R | | |
| 6h-Methioninase-6H- | AGCAATGTAGAAAGGTGTTGTTGTGATTCGTAGCA | 126 |
| E269UAG-F | | |
| 6h-Methioninase-6H- | CCTTTCTACATTGCTTTACGCATACCGGCA | 127 |
| E269UAG-R | | |
| 6h-Methioninase-6H- | CGATCCGTAGGTTATTCAAGAATATTTCCATACCT | 128 |
| K319UAG-F | | |
| 6h-Methioninase-6H- | ATAACCTACGGATCGCTGGTACGGGT | 129 |
| K319UAG-R | | |
| 6h-Methioninase- | AGCAATTTAGCATGGTGTGAAAGGTATTGTTTATG | 130 |
| Q240UAG-F | | |
| 6h-Methioninase- | CCATGCTAAATTGCTGCATCATACAGATATTCC | 131 |
| Q240UAG-R | | |
| 6h-Methioninase- | GCCTGATTAGGAACTGCCTGGTCTGGTTAGC | 132 |
| E289UAG-F | | |
| 6h-Methioninase- | AGTTCCTAATCAGGCGGAACAATACCATTACC | 133 |
| E289UAG-R | | |

The five types of constructed pTAC-6h-Methioninase variants plasmid were each independently transformed into E. coli DH5alpha strain and purified by plasmid miniprep. Then, the plasmids were digested with a KpnI restriction enzyme, and a 1221-bp insert DNA band was confirmed through electrophoresis in a 1% agarose medium.

### Experimental Example 3.2 Selection of Methioninase Variant

Using the methioninase and the variant expression vector thereof constructed according to Experimental Example 3.1, the methioninase and the variants thereof were expressed according to Experimental Examples 1.4 to 1.5 and analyzed.

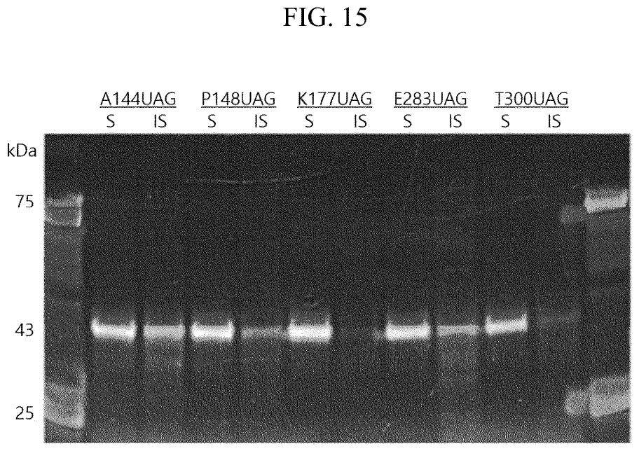

As a result of SDS-PAGE, it was confirmed that 6h-methioninase in which a nonnatural amino acid was introduced into the amino acid, proline, at position 148 or 283 exhibited the highest soluble protein expression amount and was thus selected as a target for mass production (FIG. 15).

### Experimental Example 3.3 Obtainment of Methioninase Variant

To obtain the methioninase variant selected in Experimental Example 3.2, the corresponding strain was mass-cultured according to Experimental Example 1.6, and the cells were disrupted under the following conditions.

The strain was washed three times with distilled water three times the volume with respect to the weight of the cells, and as a lysis buffer, a PBS pH 7.0 buffer was used. Ultrasonic disruption was performed two times for 20 minutes under the conditions of amplification factor: 30%, 5 sec on, and 5 sec off. After the disruption, the supernatant was collected by centrifugation (13,000×g, 30 min, and 4° C.). The collected supernatant was filtered using a 0.2-um filter.

Purification and analysis were performed on the collected disruption supernatant according to Experimental Examples 1.7.

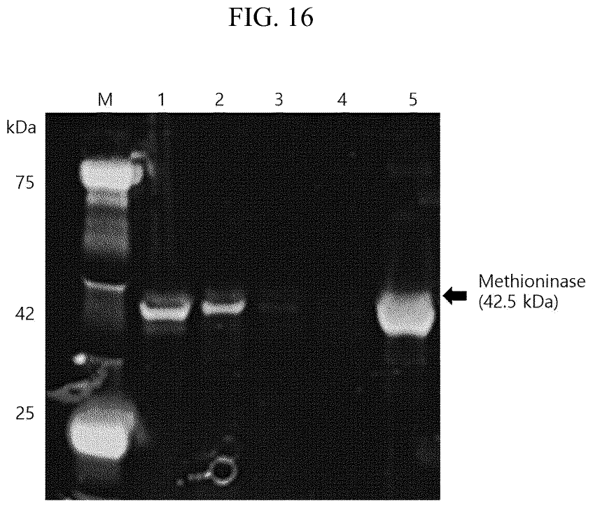

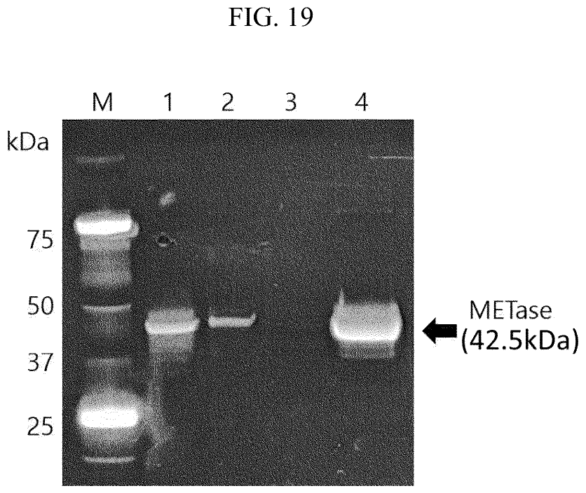

As a result of the analysis, it was confirmed that the 6H-methioninase (P148UAG and P283UAG) protein was normally eluted in the elution fractions (FIGS. 16 and 19).

### Experimental Example 3.4 Preparation of Methioninase-Albumin Conjugate

A methioninase-albumin conjugate was prepared by using the cell disruption product of Experimental Example 3.3 and the albumin-linker conjugate of Experimental Example 1.10.

Specifically, the 6H-methioninase variant was conjugated to rHA-TCO in the cell disruption supernatant itself, so not only the 6H-methioninase variant but also other unnecessary proteins were present. Thus, the protein yield of the 6H-methioninase variant in the disruption supernatant per 1 g wet cell was set to 1 mg/g wet cell. In addition, using 2 mg rHA-TCO per 1-g wet cell, 6h-methioninase-rHA conjugation was performed based on rHA at a concentration of 50 uM. The conjugation was performed under conditions: a temperature of 23° C., a speed of 130 rpm, and a duration of 16 hours.

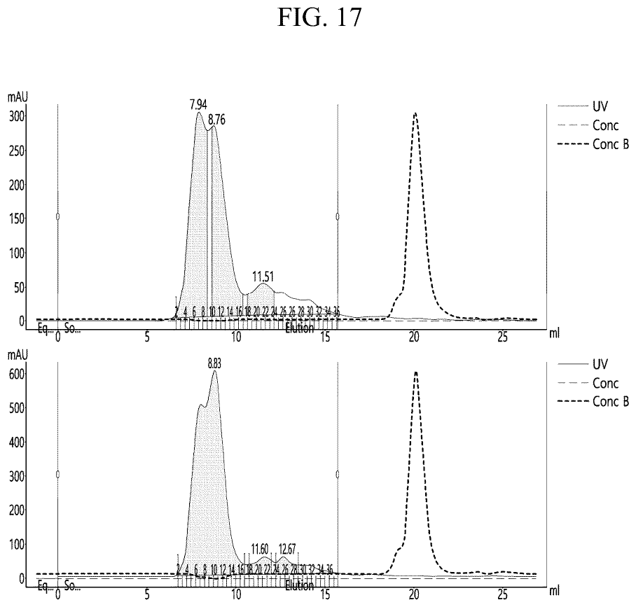

As a result of SDS-PAGE analysis, a 109 kDa protein band of the 6h-methioninase variant-rHA conjugate was confirmed (FIG. 17).

### Experimental Example 3.5 Purification of Methioninase-Albumin Conjugate

After the completion of the reaction, 6H-methioninase-rHA conjugate was filtered using a 0.2-um filter, and Ni-NTA purification was then performed according to Experimental Example 1.7. The 6H-methioninase-rHA conjugate mixture was later bound to 20-mL Ni-NTA resin at 4° C. for 1 hour, followed by performing 5 CV washing with 50 mM sodium phosphate, 300 mM NaCl, and 20 mM imidazole pH 8.0.

Elution of the target protein was performed using 50 mM sodium phosphate, 300 mM NaCl, and 300 mM imidazole pH 8.6 buffer 2 CV.

Each Ni-NTA fraction was conjugated at a final concentration of 20 uM using 10 mM methyltetrazin-Cy3 fluorescent linker stock and analyzed by SDS-PAGE. A 6H-methioninase-rHA tetramer was confirmed by SEC-HPLC analysis.

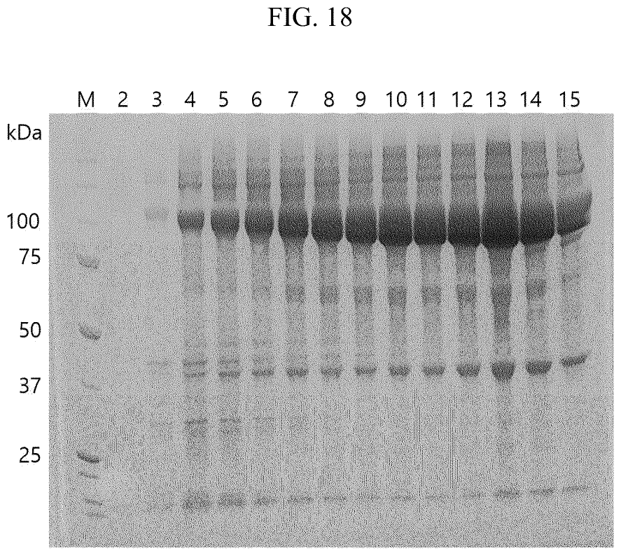

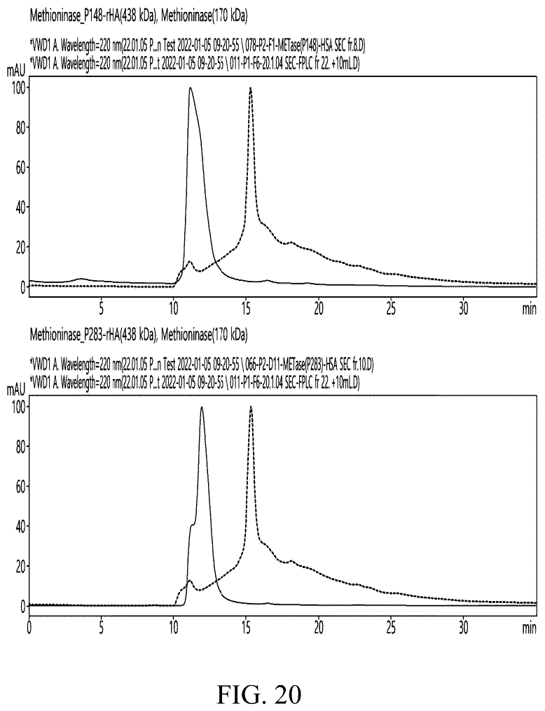

Next, SEC-FPLC purification was performed on the elution fractions according to Experimental Example 1.9. Each fraction was analyzed by SDS-PAGE. The 6H-methioninase-rHA tetramer was analyzed using SEC-HPLC (FIGS. 18 and 20).

### Experimental Example 3.6 Purification and Analysis of Wild-Type Methioninase

6h-methioninase-WT was subjected to primary purification by Ni-NTA and then conjugated to TCO-Cy3 fluorescent linker. Next, analysis was performed by SDS-PAGE (FIG. 16). Thereafter, the elution fractions were concentrated using Vivaspin (10,000 MWCO) and purified by SEC-FPLC. SEC-FPLC was performed using a PBS pH 7.4 buffer for equilibration, and through 1.1 CV elution at flow rate of 0.5 mL/min, each fraction was analyzed by SDS-PAGE and SEC-HPLC.

### Experimental Example 3.7 Evaluation of Methioninase-Albumin Conjugate Activity

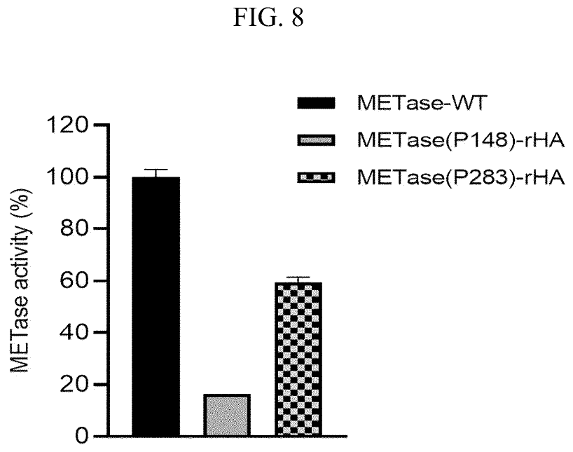

The activity of the purified methioninase-rHA tetramer was calculated by measuring the amount of 2-ketobutyrate produced by reacting L-methionine with methioninase, and the unit activity was defined as the amount of 1.0 mmole of 2-ketobutyrate produced per minute under conditions: a pH of 8.0, a temperature of 37° C., and a duration of 1 minute. In the measurement method, the purified methioninase-rHA tetramer or a blood sample was added to a 100 mM potassium phosphate buffer (pH 8.0) containing 25 mM methionine (Sigma) and 0.01 mM pyridoxal-5-phosphate (Sigma) and reacted at 37° C. for 20 minutes. Then, a 50% trichloroacetic acid solution was added to terminate methioninase activity. The supernatant (pH 5.0) separated from the reaction solution by centrifugation was mixed with a 1 M sodium acetate buffer and 0.1% 3-methyl-2-benzothiazolinone hydrazone (Alfa Aesar), reacted at 50° C. for 30 minutes, and then reacted at 25° C. for 30 minutes. The activity was calculated by measuring the absorbance of the reaction solution at 320 nm (FIG. 8).

### Experimental Example 3.8 PK Analysis of Methioninase-Albumin Conjugate

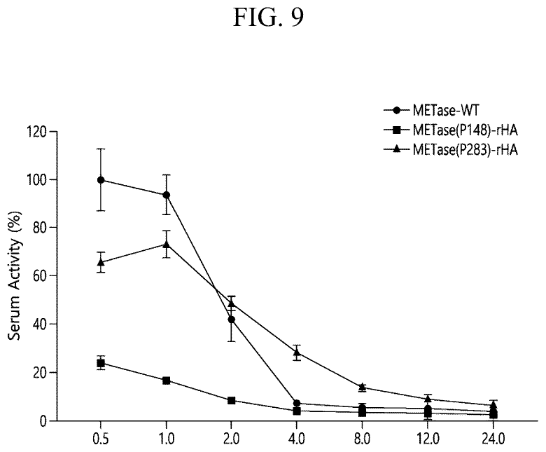

For half-life analysis of methioninase-WT and methioninase variant-rHA in vivo, 250 μL of each protein at a concentration of 3 mg/ml in PBS was injected into the tail vein of 6-week-old ICR male rats (n=6). After the injection, blood samples were collected and analyzed at 0.5, 1, 2, 4, 8, 12, and 24 hours. As a result of the analysis, it was confirmed that the half-life of the methioninase-albumin conjugate was increased by six or more times than that of wild-type methioninase (FIG. 9).

### Experimental Example 4 Comparison of Multimeric Protein-Albumin Conjugate Preparation Process

### Experimental Example 4.1 Construction of Urate Oxidase Variant Expression Vector

Using the gene encoding Uox derived from Aspergillus flavus as a template, a pTAC_Uox plasmid was constructed. To apply the TAC promoter, the sequence information of the pTAC-MAT-TAG-1 expression vector (Sigma, E5530) was referred to, and 5′-TTTGTTTAACTTTAAGAAGGAGA-3′ (SEQ ID NO: 151), a further extended ribosome binding site (RBS) sequence compared to that of the pQE80 vector, was applied. For recombinant protein expression of the pTAC vector, transcription control as well as rrnBt1 and rrnBt2 terminator sequence were applied.

A request was made to Macrogen for DNA synthesis of the rrnbT1-rrnbT2 terminator sequence from the TAC promoter, and cloning was performed on the pQE80L vector to construct a pTAC-empty vector. Cloning of the pTAC-empty vector was performed by infusion cloning, which was completed using an Infusion® HD cloning kit (Takara Korea Biomedical). A request was made for sequencing analysis of the constructed pTAC-empty vector to complete sequence verification.

Each sequence used to construct the vector is shown in Table 7 below.

TABLE 7

| | | SEQ | | GC | TM |
|---|---|---|---|---|---|
| Label | Sequence (5′ to 3′) | ID NO | bp | (%) | (° C.) |
| pTAC | CAAGCTTGGCTGTTTTGGCG | 159 | 20 | 55 | 64 |
| linearize-F | | | | | |
| pTAC | CTATATCTCCTTCTTAAAGTTAAAC | 160 | 25 | 28 | 53 |
| linearize-R | | | | | |
| tacP-RBS-MCS- | AAGAAGGAGATATAGATGTCTGCTGTGAAGGCCG | 161 | 34 | 47 | 62 |
| rrnBt1t2-F | | | | | |
| tacP-RBS-MCS- | AAACAGCCAAGCTTGTTACAGCTTGCTCTTCAGAGA | 162 | 36 | 44 | 59 |
| rrnBt1t2-R | | | | | |
| pTAC- | GCCTAGAGCAAGACGTTTCC | 163 | 20 | 55 | 57 |
| sequencing-F | | | | | |
| pTAC- | TTAATGCAGCTGGCACGAC | 164 | 19 | 53 | 58 |
| sequencing-R | | | | | |

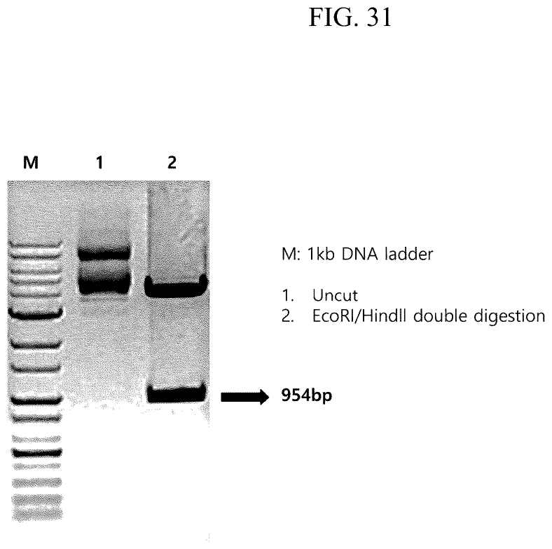

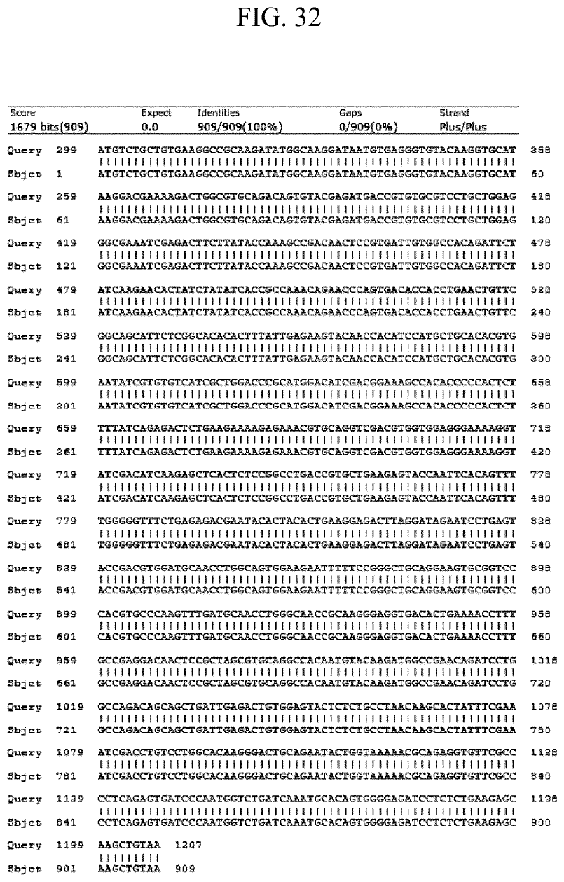

To amplify the gene encoding Uox derived from Aspergillus flavus as a template, infusion cloning was performed on the pTAC-empty vector constructed by performing PCR-amplification on the previously cloned pQE80-Uox-W174mb vector. In this case, the constructed pTAC vector is capable of expressing the urate oxidase variant of SEQ ID NO: 166, and X of the sequence is 4-(1,2,3,4-tetrazin-3-yl) phenylalanine. 50 ng of the vector and 22 ng of the insert (Uox_W174mb) were reacted during the infusion reaction, which was performed at 50° C. for 15 minutes, and then transformed into E. coli DH5a. Next, miniprep was performed by picking up and inoculating a single colony in a 4 mL LB broth medium. Then, EcoRI/HindIII restriction enzyme digestion was performed to confirm the band (953 bp) of Uox-W174mb, the cloning insert gene sequence (FIG. 31). A request was made for sequencing of the cloned pTAC-Uox-W174mb, and the cloning result was confirmed using NCBI's BLAST program (FIG. 32).

For Uox-frTet expression, C321delAexp Escherichia coli host cells were co-transformed using the pDule_C11 and pTAC_Uox-174Amb plasmids constructed in Experimental Example 4.1 (C321delA.exp [pDule_C11][pTAC_Uox-174Amb]), and the resulting product was then cultured in a 2× YT medium. Uox-frTet expression was performed using a protocol to which 1-to-3 mM frTet, tetracycline (10 μg/mL), and kanamycin (35 μg/mL) were added.

### Experimental Example 4.2 Process A of Obtaining Urate Oxidase-Albumin Conjugate

For separation and purification of Uox-frTet from the urate oxidase variant expression strain according to Experimental Example 4.1, the obtained cells were mixed with a buffer (20 mM Tris-HCl pH 9.0) at a ratio of 1:5 (w/w %), and cell disruption was performed using an ultrasonicator. After the cell disruption, centrifugation was performed at 9,500 rpm for 40 minutes to remove cell debris, and the supernatant was then collected.

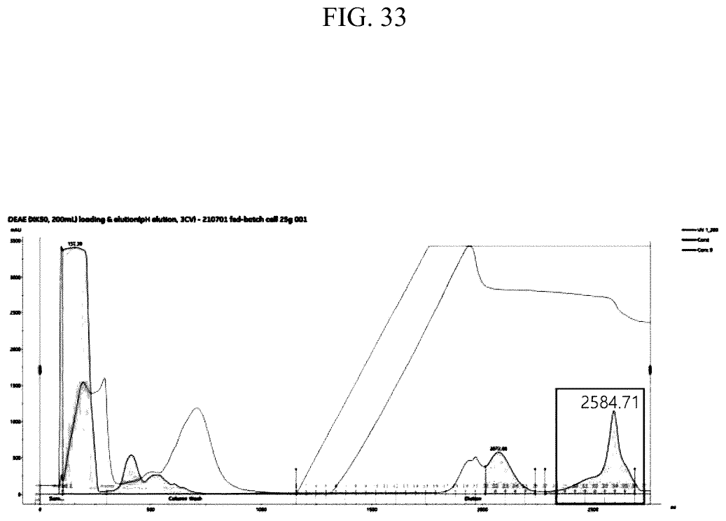

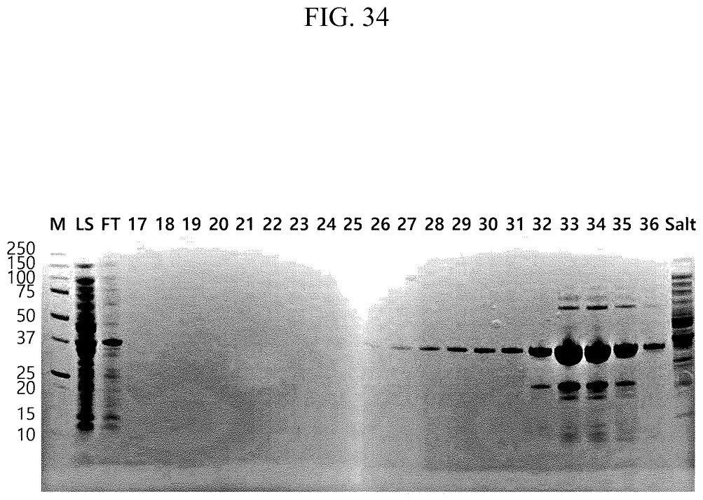

The collected supernatant was filtered using a 0.45-um filter, and primary separation and purification were then performed through a DEAE Sepharose Fast Flow (Cytiva, MA, USA) column using an equilibration buffer, 20 mM Tris-HCl pH 9.0, and an elution buffer, 20 mM sodium phosphate pH 6.0 (FIGS. 33 and 34).

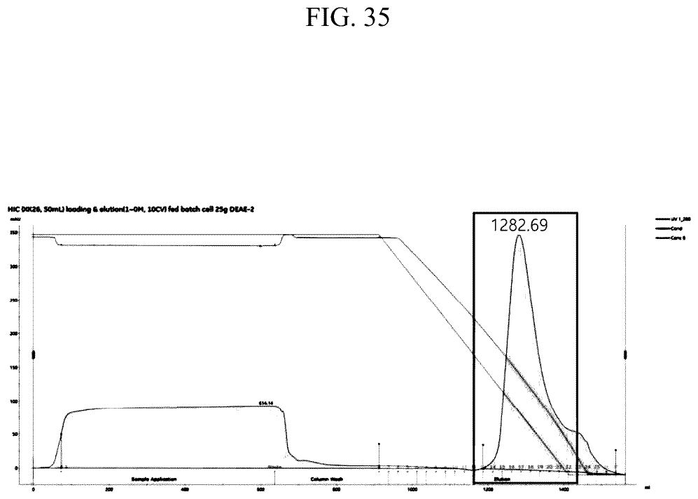

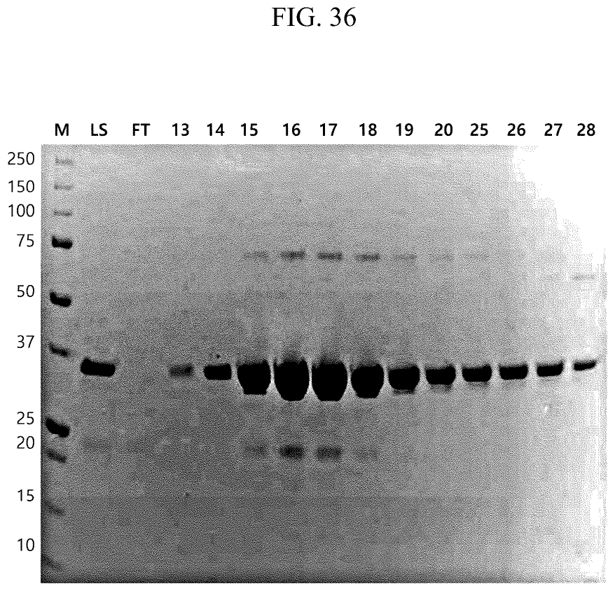

The fractions obtained by the primary purification were integrated, and then subjected to secondary separation and purification through a phenyl Fast Flow (Cytiva, MA, USA) column using an equilibration buffer, 18 mM Tris-HCl pH 9.0+1 M ammonium sulfate, and an elution buffer, 18 mM Tris-HCl pH 9.0 (FIGS. 35 and 36).

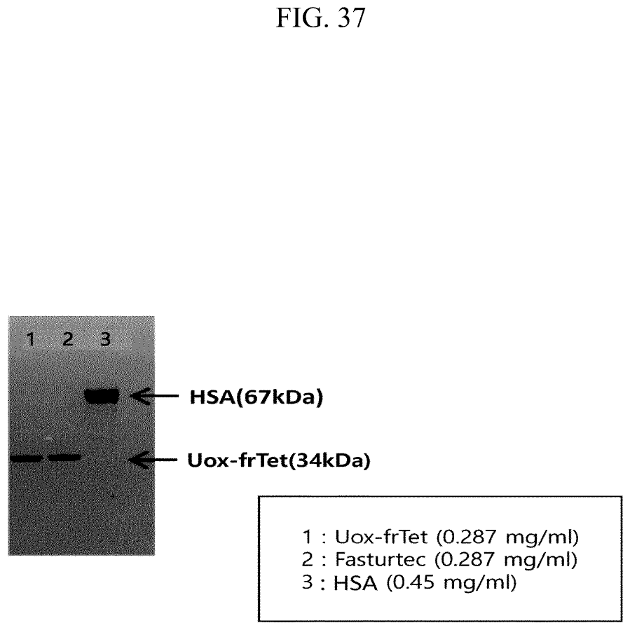

As a result of integrating and analyzing the fractions obtained by the secondary purification, highly pure Uox-frTet was obtained. In addition, after performing purification on Coomassie blue-stained protein gel, a single band with a molecular weight of about 34 kDa was present in the elution lane. Furthermore, the SDS-PAGE analysis result confirmed that there was a molecular weight band matching with FASTURTEC (Rasburicase, Sanofi-Aventis), urate oxidase currently available in the market (FIG. 37).

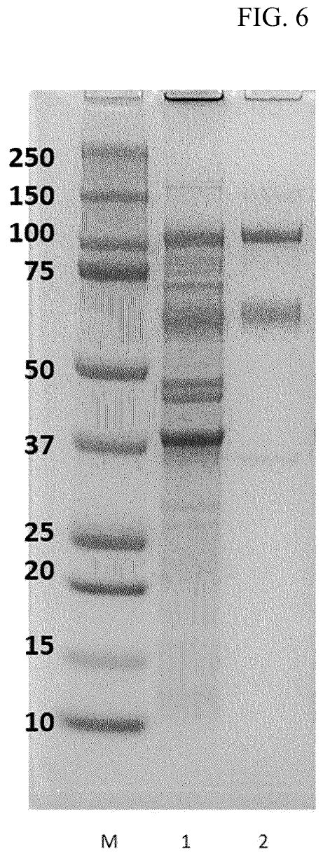

For Uox-HAS preparation, HSA and a TCO-maleimide linker were conjugated in a molar ratio of 1:4 for 3 hours at room temperature. To remove the unreacted TCO-MAL linker, the reaction mixture was removed and desalted with a PBS buffer (pH 7.0) using a HiPrep 26/10 Desalting (Cytiva, MA, USA) column. Then, HSA-TCO and Uox-frTet were reacted in a molar ratio of 4:1 for 15 hours at room temperature. The sample having undergone the reaction was filtered using a 0.45-μm filter and then analyzed on a Coomassie blue-stained gel. As a result of the analysis, a main band with a molecular weight of about 101 kDa was found to exist, which was consistent with the expected molecular weight of the monomer Uox-HSA (101 kDa) (FIG. 6). Thereafter, the sample was buffer-exchanged with 20 mM sodium phosphate pH6.0+30 mM NaCl and then injected into an SP Sepharose column (Cytiva, MA. USA) to perform primary separation and purification using an elution buffer, 18 mM sodium phosphate pH 6.0+1 M NaCl.

Thereafter, the buffer was exchanged to perform separation using a Q-HP column and then perform separation and purification using an SEC column. Lastly, 0.4 mg of Uox-HSA conjugate was obtained using 1 g of wet cells.

### Experimental Example 4.3 Process B of Obtaining Urate Oxidase-Albumin Conjugate

For separation and purification of Uox-frTet from the urate oxidase variant expression strain according to Experimental Example 4.1, the obtained cells were mixed with a buffer (20 mM Tris-HCl pH 9.0) at a ratio of 1:5 (w/w %), and cell disruption was performed using an ultrasonicator. After the cell disruption, centrifugation was performed at 9,500 rpm for 40 minutes to remove cell debris, and the supernatant was then collected.

For Uox-HSA preparation using the cell disruption supernatant, HSA and a TCO-maleimide linker were conjugated in a molar ratio of 1:4 for 3 hours at room temperature. To remove the unreacted TCO-MAL linker, the reaction mixture was removed and desalted with a PBS buffer (pH 7.0) using a HiPrep 26/10 Desalting (Cytiva, MA, USA) column. Then, HSA-TCO and Uox-frTet were reacted in a molar ratio of 4:1 for 15 hours at room temperature. The sample having undergone the reaction was filtered using a 0.45-μm filter and then analyzed on a Coomassie blue-stained gel. As a result of the analysis, a main band with a molecular weight of about 101 kDa was found to exist, which was consistent with the expected molecular weight of the monomer Uox-HSA (101 kDa) (FIG. 6). Thereafter, the sample was buffer-exchanged with 20 mM sodium phosphate pH 6.0+30 mM NaCl and then injected into an SP Sepharose column (Cytiva, MA. USA) to perform primary separation and purification using an elution buffer, 20 mM sodium phosphate pH 6.0+1 M NaCl.

The fractions obtained by the primary purification were integrated to be buffer-exchanged with a PBS buffer (pH 7.4), and secondary separation and purification were performed through a Superdex 200 96 Increase 10/300 GL (Cytiva, MA, USA) column. Lastly, 0.7 mg of Uox-HSA conjugate was obtained using 1 g of wet cells.

### Experimental Example 4.4 Process C of Obtaining Urate Oxidase-Albumin Conjugate

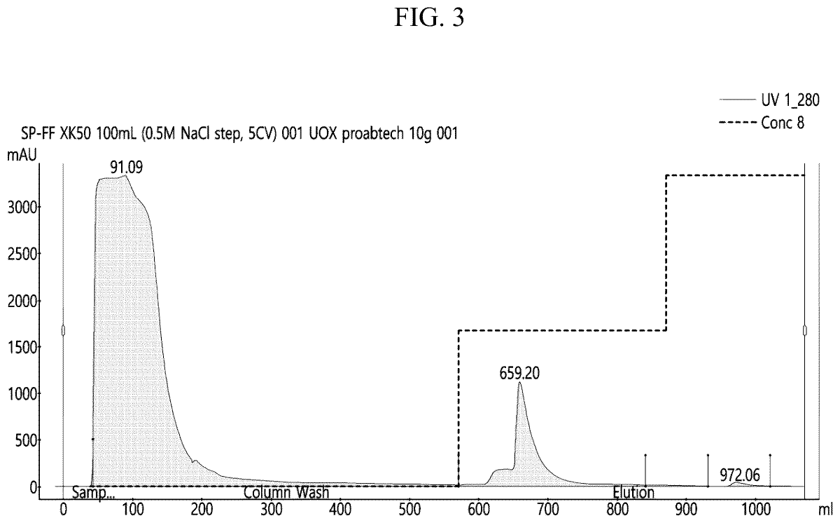

For separation and purification of Uox-frTet from the urate oxidase variant expression strain according to Experimental Example 4.1, the obtained cells were mixed with a buffer (20 mM sodium phosphate buffer pH 7.0) at a ratio of 1:5 (w/w %), and cell disruption was performed using an ultrasonicator. After the cell disruption, centrifugation was performed at 9,500 rpm for 40 minutes to remove cell debris, and the supernatant was then collected. The collected supernatant was filtered using a 0.45-um filter, and purification was then performed through a Sepharose Fast Flow (Cytiva, MA, USA) column using an equilibration buffer, 20 mM sodium phosphate buffer pH 7.0 (FIG. 3).

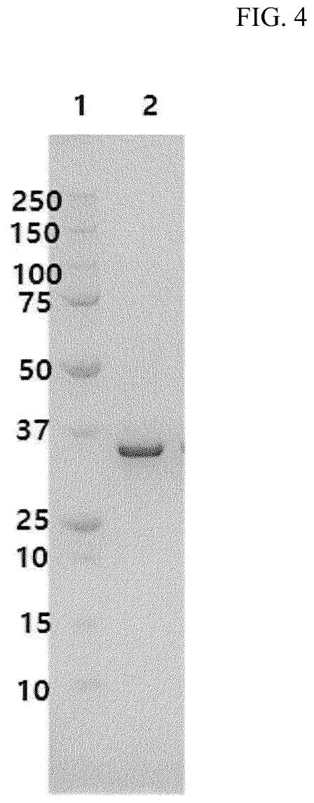

As a result of integrating and analyzing the purified fractions, highly pure Uox-frTet was obtained. In addition, after performing purification on Coomassie blue-stained protein gel, a single band with a molecular weight of about 34 kDa was present in the elution lane (FIG. 4). Furthermore, analysis was performed using high-performance liquid chromatography (HPLC) for further confirmation of purity. TSKgel 90SWXL (TOSOH) was used as the analysis column, and analysis was performed at 200-nm UV at a speed of 0.5 mL/min using a mobile phase PBS buffer (pH 7.4).

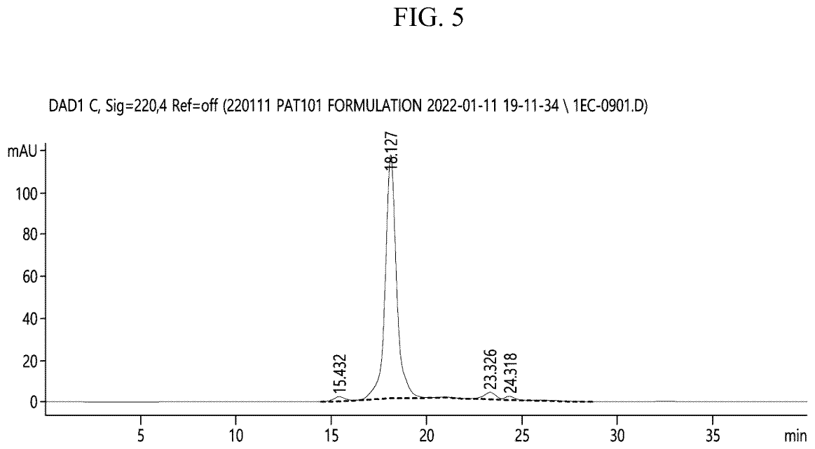

As a result, SP-purified Uox-frTet was detected as a main peak at 18.12 minutes, and the purity was observed to be 94% or higher. It was confirmed that Uox-frTet was highly purified (FIG. 5).

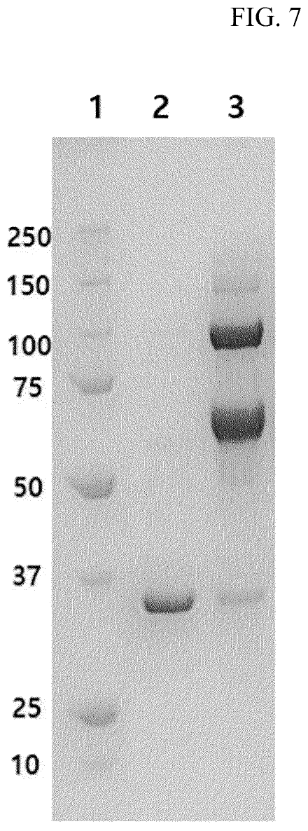

For Uox-HAS preparation, HSA and TCO-maleimide linker were conjugated in a molar ratio of 1:3 for 2 hours at room temperature. To remove the unreacted TCO-MAL linker, the reaction mixture was removed and desalted with a 20 mM sodium phosphate buffer (pH 7.0) using a HiPrep 26/10 Desalting (Cytiva, MA, USA) column. Then, HSA-TCO and Uox-frTet were reacted in a molar ratio of 5:1 for 1.5 hours at room temperature. The sample having undergone the reaction was filtered using a 0.45-μm filter and then analyzed on a Coomassie blue-stained gel. As a result of the analysis, a main band with a molecular weight of about 101 kDa was found to exist, which was consistent with the expected molecular weight of the monomer Uox-HSA (101 kDa) (FIG. 7). Thereafter, the sample was buffer-exchanged with 20 mM sodium phosphate pH 6.0+30 mM NaCl and then injected into an SP Sepharose column (Cytiva, MA. USA) to perform primary separation and purification using an elution buffer, 20 mM sodium phosphate pH 6.0+1 M NaCl.

The fractions obtained by the primary purification were integrated to be buffer-exchanged with a PBS buffer (pH 7.4), and secondary separation and purification were performed through a Superdex 200 Increase 10/300 GL (Cytiva, MA, USA) column. Lastly, 1.4 mg of Uox-HSA conjugate was obtained using 1 g of wet cells.

### Experimental Example 4.5 Comparison of Urate Oxidase-Albumin Conjugate Obtaining Processes

As a result of comparison, Process B (0.7 mg/1 g wet cell) and Process C (1.4 mg/1 g wet cell) exhibited yields that are 2 to 3.5 times higher than that of Process A (0.4 mg/1 g wet cell), an existing process.

### Experimental Example 5 Preparation and Evaluation 1 of Arthrobacter globiformis-Derived Urate Oxidase-Albumin Conjugate

### Experimental Example 5.1 Experimental Materials

4-(1,2,3,4-tetrazin-3-yl) phenylalanine (frTet) was purchased from Aldlab media (Woburn, MA, USA). Trans-cyclooctene (TCO)-Cy3 was purchased from AAT Bioquest (Sunnyvale, CA, USA). TCO-PEG4-maleimide (TCO-PEG4-MAL) and amine-axially substituted TCO (TCO-amine) were purchased from FutureChem (Seoul, Korea). Pentafluorophenyl ester (PFP)-PEG4-APN was purchased from CONJU-PROBE (San Diego, CA, USA). Disposable PD-10 desalting columns and Superdex 200 10/300 GL Increase columns were purchased from Cytiva (Uppsala, Sweden). Vivaspin 6 centrifugal concentrators with molecular weight cut-offs (MWCOs) of 10 and 100 kDa were purchased from Sartorius (Gottingen, Germany). Human serum albumin (HSA) and all other chemical reagents were purchased from Sigma-Aldrich unless otherwise stated.

### Experimental Example 5.2 Vector Construction for Obtaining Arthrobacter globiformis-Derived Urate Oxidase Variant (AgUox-frTet) Derived from

A request was made to Macrogen (Seoul, South Korea) for synthesizing gene encoding Arthrobacter globiformis-derived urate oxidase (AgUox) and variants thereof. To express wild-type AgUox (AgUox-WT) or an AgUox variant (AgUox-frTet) in which frTet, a nonnatural amino acid, was introduced into the sequence, the synthesized gene was used as a template and amplified by polymerase chain reaction (PCR) using primers, pBAD-AgUox_F (5′-GCCGCCATGGTGTCTGCTGTGAAGG-3′, SEQ ID No: 17) and pBAD-AgUox_R (5′-GCCGAGATCTTTAATGGTGATGGTG-3′, SEQ ID No: 162). The amplified gene was digested with two restriction enzymes (NcoI and BglIII) and then introduced into the NcoI and BgIII sites of the pBAD vector to synthesize pBAD_AgUox. To replace the glutamic acid codon at position 196 in the AgUox-WT sequence with the amber codon (UAG), the pBAD-AgUox was used as a template, and primers, AgUox-196Amb_F (5′-GTCGAAGTCCACCTATACGGTGTTGTAACGCCAACGG-3′, SEQ ID No: 163) and AgUox-196Amb_R (5′-CCGTTGGCGTTACAACACCGTATAGGTGGACTTCGAC-3′, SEQ ID No: 164), were used to construct pBAD-AgUox 196amb.

### Experimental Example 5.3 Obtainment of AgUox-frTet

For AgUox-frTet expression, with reference to the method disclosed in Yang et.al, Temporal Control of Efficient In Vivo Bioconjugation Using a Genetically Encoded Tetrazine-Mediated Inverse-Electron-Demand Diels-Alder Reaction, Bioconjugate Chemistry, 2020, 2456-2464, C321ΔA.exp, pDule_C11, and pBAD_AgUox-196amb were used to express AgUox-frTet in the following manner. E. coli cells containing frTet-optimized MjtRNA^{TYr}/MjTyrRS were prepared. E. coli cells cultured in a Luria Broth (LB) medium containing ampicillin (100 pg/mL) and tetracycline (10 μg/mL) were shaken overnight at 37° C. while being inoculated in a 2× YT medium under the same conditions. After 2.5 hours of shaking culture, when the medium containing the cells reached an optical density of 0.5 at 600 nm, frTet and L-(+)-arabinose were added to the medium at final concentrations of 1 mM and 0.4% (w/w), respectively. After culturing for 5 hours, the cells were centrifuged under conditions: a speed of 5000 rpm, a temperature of 4° C., and a duration of 10 minutes. Then, AgUox-frTet was obtained. The AgUox-frTet was purified by immobilized metal affinity chromatography at 4° C. according to the manufacturer's protocol (Qiagen). The purified AgUox-frTet was desalted with PBS (pH 7.4) using a PD-10 column. AgUox-WT expression and purification processes were performed in a similar manner to the expression and purification of AgUox-frTet without adding tetracycline and frTet to the culture medium during the expression step.

### Experimental Example 5.4 Analysis and Confirmation of Prepared AgUox-frTet

To confirm the prepared AgUox-frTet and AgUox-WT, they were digested with trypsin according to the manufacturer's protocol. A total of 0.4 mg/mL of the Uox variants (AgUox-WT and AgUox-frTet) were digested at 37° C. overnight and then desalted using a ZipTip C18. The mixture digested with trypsin was mixed with a 2,5-dihydroxybenzoic acid (DHB) matrix solution (30:70 (v/v) acetonitrile: 20 mg/mL of DHB in 0.1% trifluoroacetic acid) and then analyzed using Microflex MALDI-TOF/MS instrument (Bruker Corporation, Billerica, MA, USA).

### Experimental Example 5.5 Fluorescent Dye Labeling for Each Site of AgUox-WT and AgUox-frTet

Purified AgUox-WT and AgUox-frTet were reacted with TCO-Cy3 fluorescent dye in a molar ratio of 1:2 in PBS (pH 7.4) at room temperature. After 2 hours, the reaction mixture was subjected to sodium dodecyl sulfate-polyacrylamide gel electrophoresis (SDS-PAGE). Fluorescence images of protein gel were obtained using the ChemiDoc XRS+ system (illumination at 302 nm, 510-70 nm filter, Bio-Rad Laboratories, Hercules, CA, USA). After fluorescence analysis, the protein gel was stained with Coomassie Brilliant Blue R-250 dye. Using the ChemiDoc XRS+ system with white light illumination, protein gel images were obtained.

### Experimental Example 5.6 Preparation of AgUox-HSA Conjugates (AgUox-MAL-HSA and AgUox-APN-HSA)

To perform site-specific albumination of AgUox, HSA was purified by anion-exchange chromatography through a HiTrap Q HP anion-exchange column. The purified HSA was desalted with PBS (pH 7.0) and then reacted with TCO-MAL at a molar ratio of 1:4 in PBS (pH 7.0) at room temperature. After 2 hours, the reaction mixture was desalted with PBS (pH 7.4) through a PD-10 column, and the unreacted TCO-PEG4-MAL linker was removed to obtain a MAL-HSA conjugate. The purified Uox-frTet was reacted with MAL-HSA in a molar ratio of 1:4 in PBS (pH 7.4) at room temperature for 5 hours. After conjugation, the reaction mixture was applied to size-exclusion chromatography (SEC) using the NGC Quest 10 Plus chromatography system (Bio-Rad Laboratories Inc., Berkeley, CA, USA). The molecular weight and purity of the elution fractions were analyzed by SDS-PAGE, and the fractions corresponding to AgUox-frTet conjugated to four MAL-HSA molecules (AgUox-MAL-HSA) were selected and concentrated for further analysis. To prepare an AgUox-HSA conjugate using a hetero-bifunctional cross-linker containing TCO and APN, TCO-amine was reacted with PFP-PEG4-APN in a molar ratio of 1:1 in DMSO for 30 minutes at room temperature. The purified HSA was buffer-exchanged with a 50 mM sodium borate buffer (pH 9.0). In the 50 mM sodium borate buffer (pH 9.0), the purified HSA was reacted with TCO-PEG4-APN in a molar ratio of 1:4 at room temperature for 2 hours. To remove the unreacted TCO-APN linker, the reaction mixture was desalted with PBS (pH 7.4) using a PD-10 column. Conjugation (AgUox-APN-HSA) and purification of AgUox-frTet conjugated to four HSA molecules through an APN-containing linker were performed in a similar manner to conjugation and purification of AgUox-MAL-HSA.

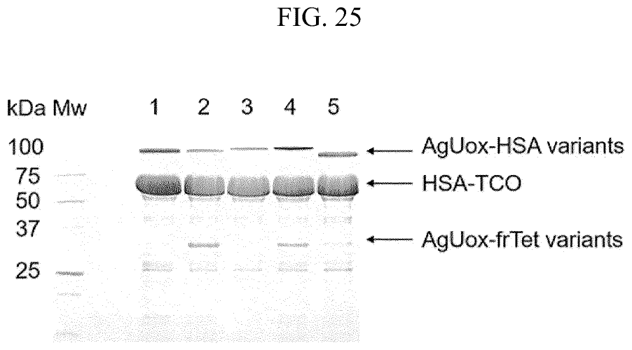

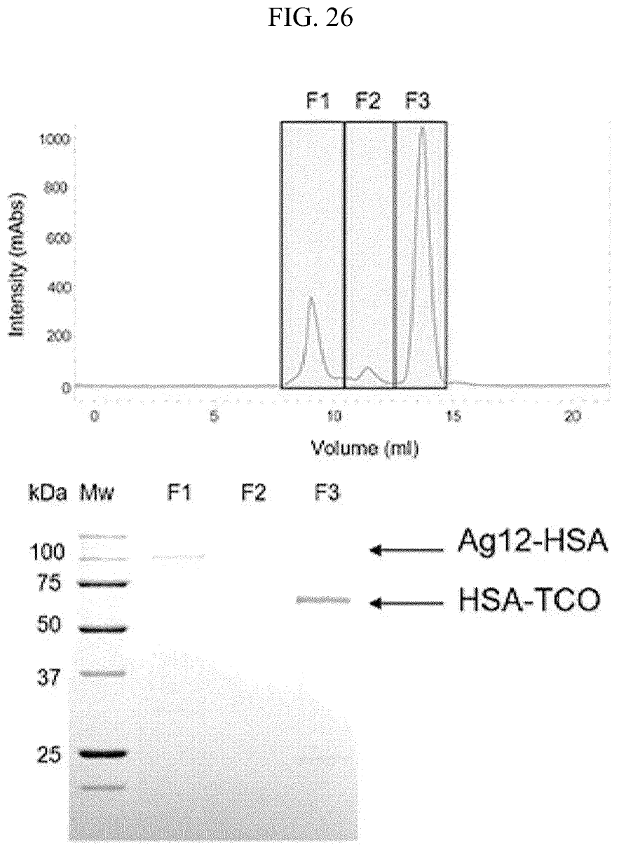

The results of preparing the AgUox-HSA conjugate according to Experimental Example 5.5 are shown in FIGS. 25 to 26.

### Experimental Example 5.7 Confirmation of In Vivo Half-Life Of AgUox-HSA Conjugate (PK Profile)

Stability analysis of the AgUox-HSA conjugate in mice was performed according to the guidelines of the Animal Care and Use Committee of the Gwangju Institute of Science and Technology (GIST-2020-037). Based on AgUox in 200 μL PBS, 5.0 nmol of AgUox-WT, AgUox-MAL-HSA, or AgUox-APN-HSA, was each independently injected into the tail vein of young female BALB/c mice (n=4) at pH 7.4. Blood samples were taken through retro-orbital bleeding after 15 minutes, 3 hours, 6 hours, and 12 hours in the case of AgUox-WT, and after 15 minutes, 12 hours, 24 hours, 48 hours, 72 hours, 84 hours, 96 hours, 108 hours, and 120 hours in the same manner in the case of the AgUox-HSA conjugate. After isolating serum from the collected blood, serum activities of AgUox-WT, AgUox-MAL-HSA, and AgUox-APN-HSA were measured by the change in absorbance at 293 nm when adding 100 μL of an enzyme activity assay buffer containing 100 μM uric acid to 100 μL of an enzyme activity buffer containing 5 μL of the serum.

The results of confirming the in vivo half-life are also shown in FIG. FFF. As a result of the experiment, it was confirmed that AgUox-APN-HSA and AgUox-MAL-HSA exhibited significant increases in half-life compared to AgUox-WT not involving albumin conjugation.

### Experimental Example 6 Preparation and Evaluation 2 of Urate Oxidase-Albumin Conjugate Derived from Arthrobacter globiformis

### Experimental Example 6.1 Experimental Materials

Bacto tryptone and yeast extract were purchased from BD Biosciences (San Jose, CA, USA) for use. Ni-nitrilotriacetic acid (NTA) agarose was purchased from Qiagen (Hilden, Germany) for use, and frTet [4-(1,2,3,4-tetrazin-3-yl)phenylalanine]was purchased from Aldlab media (Woburn, MA, USA). TCO-Cy3 was purchased from AAT Bioquest (Sunnyvale, CA, USA) for use. Axially substituted trans-cyclooctene-maleimide (TCO-maleimide, A) was purchased from FutureChem (Seoul, Korea). Disposable PD-10 desalting columns, HiTrap Q HP anion-exchange columns, and Superdex 200 10/300 GL size-exclusion columns were purchased from Cytiva (Uppsala, Sweden). Reagents other than the reagents described above were purchased from Sigma-Aldrich (St. Louis, Missouri, USA) for use.

### Experimental Example 6.2 Analysis of frTet Incorporation Site in AgUox

The frTet incorporation site screening was performed using the molecular modularization software PyRosetta (Python-based Rosetta molecular modeling package), which performed point mutation and energy scoring functions based on the AgUox structure (PDB ID: 2YZE). The amino acid sequence of wild-type (WT) AgUox was replaced with the Y (tyrosine) or W (tryptophan) sequence, and then the total atomic energy of the protein was calculated. The energy function of PyRosetta is based on Anfinsen's hypothesis that native protein conformations represent unique, low-energy, and thermodynamically stable conformations. The score value represents the sum of van der Waals force, attractive force, repulsive energy, Gaussian exclusion implicit solvation, and hydrogen bonds (short, long range, backbone-side chains, and side chains) between atoms on different residues separated by distance.

### Experimental Example 6.3 Preparation of Plasmids for Expression of AgUox-WT and AgUox-frTet Variants

AgUox was synthesized by Macrogen (Seoul, Korea), cloned into pBAD, and subjected to site-specific frTet incorporation, thereby generating a pBAD_AgUox plasmid. To replace sites selected by PyRosetta scoring with amber codons, site-directed mutagenesis PCR was performed using the pBAD_AgUox vector as a template. The primers used are shown in Table 8.

TABLE 8

| | | SEQ ID | | SEQ |
|---|---|---|---|---|
| Label | Forward (5′→3′) | NO | Reverse (3′→5′) | ID NO |
| AgUox_80Amb | CGTGGCGAAACCCTAACGCGCGAA | 72 | GTACGCGTTCGCGCGTTAGGG | 86 |
| (Ag1) | CGCGTAC | | TTTCGCCACG | |
| AgUox_82Amb | TTCAGTCGTGGCCTAACCATCACG | 73 | CGTTCGCGCGTGATGGTTAGG | 87 |
| (Ag2) | CGCGAACG | | CCACGACTGAA | |
| AgUox_100Amb | CCACCGGTAACCCAGTCCTAGCCT | 74 | ACACTTTACCGAAGGCTAGGA | 88 |
| (Ag3) | TCGGTAAAGTGT | | CTGGGTTACCGGTGG | |
| AgUox_101Amb | CACCGGTAACCCACTAAAAGCCTT | 75 | GGGCAAACACTTTACCGAAGG | 89 |
| (Ag4) | CGGTAAAGTGTTTGCCC | | CTTTTAGTGGGTTACCGGTG | |
| AgUox_114Amb | TACGGTCCCACTAGAACTGCTGGG | 76 | TGGGCTGCCCAGCAGTTCTAG | 90 |
| (Ag5) | CAGCCCA | | TGGGACCGTA | |
| AgUox_119Amb | ATGCATGGTCGTGGTCCTAGATAC | 77 | CTTCTGGGACCGTATCTAGGA | 91 |
| (Ag6) | GGTCCCAGAAG | | CCACGACCATGCAT | |
| AgUox_120Amb | GAGAATGCATGGTCGTGCTAGTTG | 78 | TCTGGGACCGTATCAACTAGC | 92 |
| (Ag7) | ATACGGTCCCAGA | | ACGACCATGCATTCTC | |
| AgUox_142Amb | CAGCTACGATCGCCTGTTCCTAAC | 79 | GTACTGGAAATCTCTGGTTAG | 93 |
| (Ag8) | CAGAGATTTCCAGTAC | | GAACAGGCGATCGTAGCTG | |
| AgUox_143Amb | GCCAGCTACGATCGCCTGCTAAGA | 80 | CTGGAAATCTCTGGTTCTTAG | 94 |
| (Ag9) | ACCAGAGATTTCCAG | | CAGGCGATCGTAGCTGGC | |
| AgUox_175Amb | ACGGTCGGTGGTTTCCTACAGCGT | 81 | ATAAATATACCACGCTGTAGG | 95 |
| (Ag10) | GGTATATTTAT | | AAACCACCGACCGT | |
| AgUox_195Amb | CAGCGTCGAAGTCCACTTCCTAGG | 82 | CCGTTGGCGTTACAACACCTA | 96 |
| (Ag11) | TGTTGTAACGCCAACGG | | GGAAGTGGACTTCGACGCTG | |
| AgUox_196Amb | GTCGAAGTCCACCTATACGGTGTT | 83 | CCGTTGGCGTTACAACACCGT | 97 |
| (Ag12) | GTAACGCCAACGG | | ATAGGTGGACTTCGAC | |
| AgUox_218Amb | CAGGGCCAGGGACTAAGTTTCTGC | 84 | TGCTGAAAGCATTCGCAGAAA | 98 |
| (Ag13) | GAATGCTTTCAGCA | | CTTAGTCCCTGGCCCTG | |
| AgUox_238Amb | CTTGATTTCGTCAATTTCCTAGTG | 85 | CGCGGTTATCGAGACCCACTA | 99 |
| (Ag14) | GGTCTCGATAACCGCG | | GGAAATTGACGAAATCAAG | |

### Experimental Example 6.4 Expression and Purification of AgUox-WT and AgUox-frTet Variants

For site-specific incorporation of frTet into AgUox, each variant plasmid was transformed into C321Δ.exp [pDule C11RS] competent cells to produce C321Δ.exp [pDuleC11RS][pBAD_AgUox_Amnb variant]E. coli cells. The transformants were cultured overnight under a condition of 37° C. in a Luria broth medium containing ampicillin (100 μg/mL) and tetracycline (10 μg/mL). The pre-cultured E. coli cells were inoculated into the same fresh medium. To induce protein expression, frTet and arabinose at final concentrations of 1 mM and 0.4%, respectively, were each independently added to the medium to reach an optical density of 0.5% (600 nm). After incubating the culture medium at 37° C. for 5 hours with shaking, the product was obtained by centrifugation under conditions: a temperature of 4° C., a duration of 10 minutes, and a speed of 5,000 rpm. AgUox-containing frTet variants were purified by immobilized metal affinity chromatography through the interaction between Ni-NTA and His-tag according to the manufacturer's protocol. Expression and purification of AgUox-WT were performed in a similar manner to the expression and purification of the AgUox-frTet variants without adding tetracycline and frTet.

### Experimental Example 6.5 Matrix-Assisted Laser Desorption/Ionization Time-of-Flight Mass Spectrometry (MALDI-TOF MS) and Dye Labeling of AgUox Variants

The purified AgUox-WT and AgUox-frTet variants (0.4 mg/mL) were digested with trypsin according to the manufacturer's protocol and then desalted using ZipTip C18. The trypsin-digested protein fragments were mixed with a 2,5-dihydroxybenzoic acid solution in a ratio of 1:1. The resulting mixture was loaded onto a target plate (Bruker Corporation, Billerica, MA, USA), and molecular weight was then analyzed by MALDI-TOF MS (Bruker).

To confirm the IEDDA reactivity of the AgUox-frTet variants, the purified AgUox-WT and AgUox-frTet variants were desalted with phosphate-buffered saline (PBS, pH 7.4) and then mixed with TCO-Cy3 in a molar ratio of 1:2 for 2 hours at room temperature. Thereafter, the mixture with or free of TCO-Cy3 was separated by SDS-PAGE. Gels were visualized after performing fluorescence analysis (excitation: 302 nm, filter 510/70 nm) using the ChemiDoc XRS+ system (Bio-Rad Laboratories, Hercules, CA, USA) and then Coomassie brilliant blue (CBB) staining.

### Experimental Example 6.6 Enzyme Activity Assay and Thermal Stability Evaluation of AgUox-WT and AgUox-frTet Variants

AgUox variants (100 μL of 120 nM an enzyme activity assay buffer [50 mM sodium borate and 150 mM NaCl]) were mixed with 100 μL of 180 μM uric acid in the enzyme activity assay buffer. Then, the digestion of uric acid was measured using the absorbance of the mixed solution at 293 nm. The serum activities of AgUox-WT and AgUox-frTet variants were measured by an enzyme activity assay of serum diluted in the enzyme assay buffer containing uric acid. That is, 10 μL of serum briefly isolated from whole blood was diluted in 90 μL of the enzyme activity assay buffer and then mixed with 100 μL of 200 μM uric acid solution, followed by measuring the absorbance at 293 nm. To measure the thermal stability of the AgUox variants, each variant was incubated in PBS (pH 7.4) for 10 days, and the enzyme activity assay described above was performed on days 0, 5, and 10.

### Experimental Example 6.7 Production of HSA-Conjugated AgUox-frTet Variants

High-molecular-weight aggregates were removed from human serum albumin (HSA) using anion-exchange chromatography (Hitrap Q HP column) in 18 mM a Tris buffer (pH 7.0) as previously reported. The purified HSA was desalted with PBS (pH 7.0) and reacted with a TCO-MAL heterobifunctional cross-linker in a molar ratio of 1:4 at room temperature for 2 hours. Then, the resulting mixture was desalted with PBS (pH 7.4) to produce an HSA-TCO conjugate. The purified AgUox-frTet variant was mixed with HSA-TCO in a molar ratio of 1:4 at room temperature for 5 hours, and then analyzed by SDS-PAGE to confirm a site-specific albumin conjugation yield. For further activity and pharmacokinetic studies, HSA-conjugated AgUox-196frTet (AgUox-196HSA) was isolated from the reaction mixture using size-exclusion chromatography. The elution peak corresponding to the Uox-HSA conjugate was analyzed by SDS-PAGE for molecular weight measurement and then used for an enzyme activity assay and pharmacokinetic studies.

### Experimental Example 6.8 Pharmacokinetic Studies

4.4 nmol (based on monomeric AgUox) of AgUox-WT or AgUox-HSA4 in 200 μL of PBS (pH 7.4) was intravenously injected into the tail of young female BALB/c mice (n=4). For blood half-life measurements in mice, blood samples were obtained at 15 minutes, 3 hours, 6 hours, and 12 hours after injection of AgUox-WT samples and at 15 minutes, 3 hours, 6 hours, 12 hours, 24 hours, 48 hours, and 72 hours after injection of AgUox-HSA samples by retro-orbital bleeding. An enzyme activity assay was performed to quantify residual AgUox-WT or AgUox-HSA4 in serum. The enzyme activity assay was measured in serum isolated after spinning the collected whole blood samples. Serum activity was measured in serum isolated from other collected whole blood samples.

### Experimental Example 6.9 Preparation of AgUox and AgUox-WT Containing frTet (AgUox-frTet) Variants

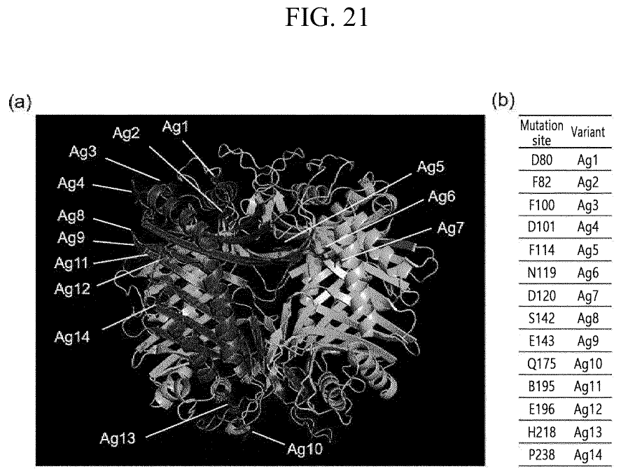

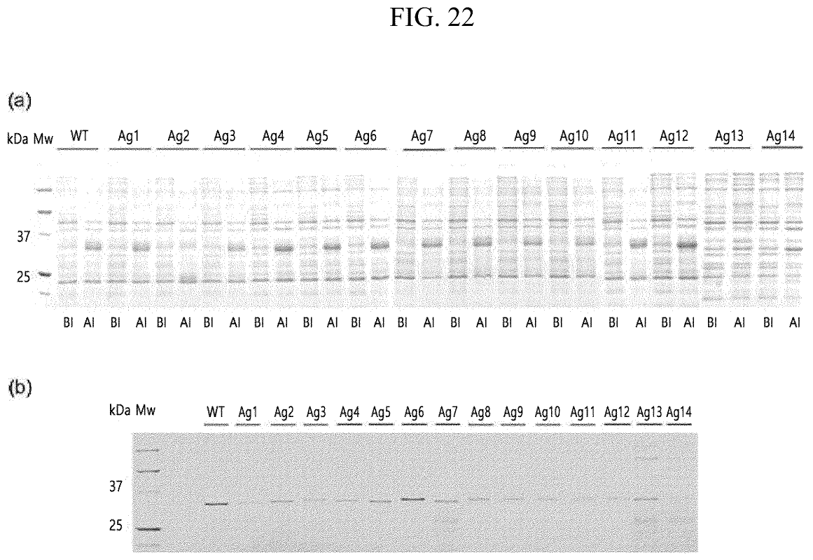

To prepare HSA-conjugated AgUox variants, the optimal sites of AgUox for HSA to conjugate were first determined. Mutations cause misfolding or unfolding of a protein. Thus, energy scores of AgUox variants containing a single mutation were calculated using PyRosetta. The energy score of each variant was converted to relative folding stability. Like the mutation to frTet (a phenylalanine analog), mutation to Y or W was introduced into various sites of AgUox-WT. Energy scores for the mutations to Y and W were confirmed through 14 AgUox variants (Ag1-14, FIG. 21) containing AgUox-WT (Table 9) and frTet (AgUox-frTet). To prepare 14 AgUox-frTet variants, an amber codon was each independently introduced into the 14 sites of AgUox-WT by PCR-mediated mutagenesis. Then, C321delAexp E. coli cells were co-transformed into the pDule C11RS plasmid encoding the MjtRNATyr/MjTyrRS specifically engineered for ftTet as well as the vectors containing each AgUox variant. The transformants were cultured to express each AgUox-frTet variant as described above in experimental example 5.2. In the CBB-stained protein gel, a molecular weight of 34 kDa, corresponding to the monomeric AgUox, was detected in cell lysate lanes after inducing and purifying AgUox-frTet (FIG. 22). Overall, such results mean that the AgUox-WT and AgUox-frTet variants were successfully expressed and purified. Table 9 shows the ROSETTA scores of AgUox after point mutation to (a) tryptophan or (b) tyrosine.

TABLE 9

| (a) | | (b) | |
|---|---|---|---|
| Mutation site | ROSETTA score | Mutation site | ROSETTA score |
| E196 | 2582.93 | E196 | 2596.28 |
| E143 | 2535.44 | E143 | 2547.95 |
| WT | 2523.29 | N119 | 2526.71 |
| P238 | 2518.78 | WT | 2523.29 |
| F114 | 2508.66 | F100 | 2517.66 |
| B195 | 2505.66 | D101 | 2517.16 |
| N119 | 2501.68 | F114 | 2514.91 |
| S142 | 2501.24 | F82 | 2511.9 |
| H218 | 2499.75 | Q175 | 2511.29 |
| Q175 | 2498.62 | P238 | 2511.12 |
| D80 | 2490.78 | S142 | 2509.92 |
| D120 | 2486.32 | D80 | 2507.26 |
| F100 | 2479.7 | D120 | 2503.98 |
| D101 | 2479.7 | H218 | 2499.57 |
| F82 | 2408.17 | B195 | 2145.17 |

### Experimental Example 6.10 Enzyme Activity and Thermal Stability Analysis of AgUox Variants

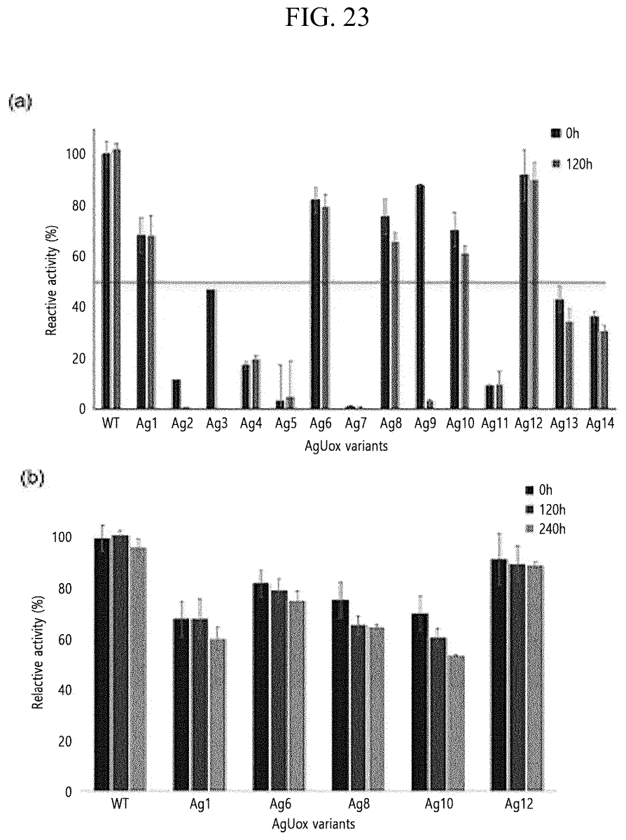

To examine whether the incorporation sites of frTet into AgUox affected biological functions, the enzyme activities of the purified AgUox-WT and AgUox-frTet variants were compared. The enzyme activities of the AgUox-frTet variants varied between 1% and 93% compared to that of AgUox-WT (FIG. 23A), indicating that the frTet incorporation sites had a significant effect on the AgUox functions. The AgUox-frTet variants, Ag1, Ag6, Ag8, Ag10, and Ag12, exhibited relatively high enzyme activities (FIG. 23A). The active sites of AgUox are present at the interface between the monomers. The frTet incorporation sites of such variants (Ag1, Ag6, Ag8, Ag10, and Ag12) are spaced apart from the active sites and interface between the monomers.

AgUox-WT was reported to be thermally stable. To evaluate the thermal stability of the AgUox-frTet variants, an enzyme activity assay was performed after culturing the AgUox-frTet variants and AgUox-WT at 37° C. for 5 days. As a result, no loss of activity of AgUox-WT was observed after 5 days of culture, as expected, (FIG. 23A). Of all the 14 AgUox-frTet variants, Ag1, Ag6, Ag8, Ag10, and Ag12 exhibited activities 50% or higher of the activity of AgUox-WT after the same period had elapsed (FIG. 23A). In the case of these 5 variants, after culturing at 37° C. for up to 10 days, Ag1, Ag6, Ag8, and Ag10 still showed activities higher than 50% of the activity of AgUox-WT (FIG. 23B). After 10 days of culture, the Ag12 variant maintained enzyme activity not inferior to that of AgUox-WT, although a slight loss of activity was observed (FIG. 23B).

### Experimental Example 6.11 Confirmation of Site-Specific frTet Incorporation into AgUox

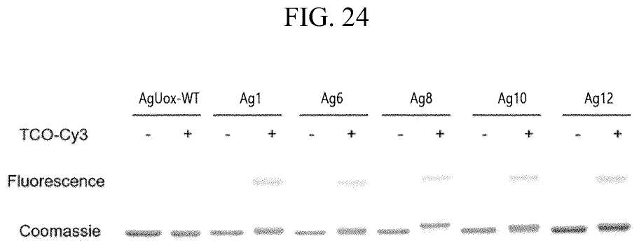

To confirm frTet incorporation into each site of AgUox, fluorescent dye labeling was performed on intact AgUox-frTet variants. As a representative example, 5 AgUox-frTet variants (Ag1, Ag6, Ag8, Ag10, and Ag12) with relatively high activity were analyzed. First, to confirm the IEDDA reactivities of the AgUox-frTet variants, fluorescent dye labeling was performed using TCO-Cy3. As a result of evaluating the fluorescence image of the protein gel, no band appeared in the AgUox-WT sample, indicating that AgUox-WT had no IEDDA reactivity (FIG. 24). On the other hand, Ag1, Ag6, Ag8, Ag10, and Ag12 variants clearly showed bands in both the fluorescent image of the protein gel and the CBB-stained protein gel, thereby confirming the IEDDA reactivity of the AgUox-frTet variant (FIG. 24).

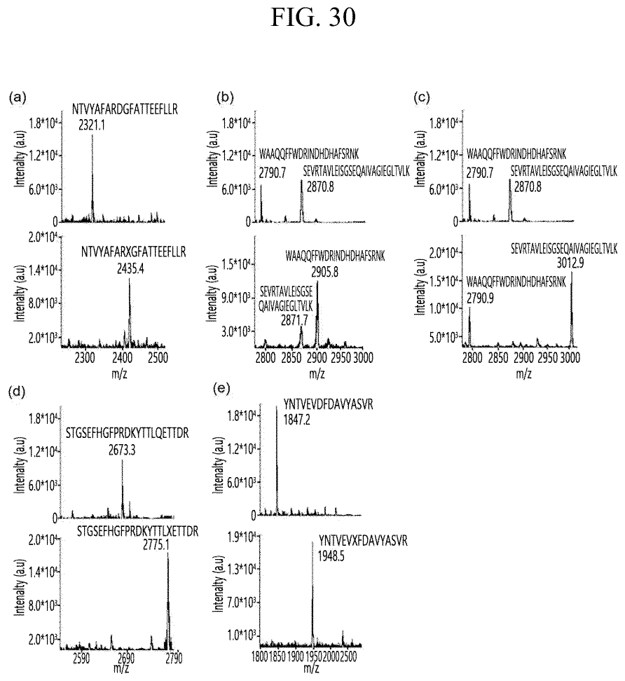

Next, frTet incorporation was further confirmed by MALDI-TOF MS of trypsin-digested AgUox-frTet (Ag1, Ag6, Ag8, Ag10, and Ag12) variants using AgUox-WT as a control. In the case of Ag1 (AgUox-D80frTet), the aspartic acid at position 80 (D80) was contained in the peptide NTVYAFARDGFATTEEFLLR (SEQ ID NO: 167, residues 72-91, theoretical mass 2,321.2 m/z) of trypsin-digested AgUox-WT. In the mass spectrum of AgUox-WT, a peak at 2,321.1 m/z consistent with the theoretical value was detected (FIG. 30A, top). In the mass spectrum of trypsin-digested Ag1, a peak (2,435.4 m/z) corresponding to the peptide NTVYAFARXGFATTEEFLLR (SEQ ID NO: 168, X=frTet) was confirmed, which was consistent with the theoretical value (2,435.3 m/z) (FIG. 30A, bottom). Substitution of D80 with frTet in the peptide resulted in a mass shift of 114.3 m/z, which was consistent with the theoretical mass shift of 114.1 m/z. Asparagine at position 119 (N119) and serine at position 142 (S142) were contained in the peptide WAAQQFFWDRINDHDHAFSRNK (SEQ ID NO: 168, residues 108-129, theoretical mass 2,789.3 m/z) and the peptide SEVRTAVLEISGSEQAIVAGIEGLTVLK (SEQ ID NO: 169, residues 130-157, theoretical mass 2,869.6 m/z). Peaks of 2,790.7 and 2,870.8 m/z were detected in the mass spectrum of AgUox-WT, which were consistent with the theoretical values (FIGS. 30B and 30C, top). In the mass spectra of trypsin-digested Ag6 (AgUox-N119frTet) and Ag8 (AgUox-S146frTet), peaks corresponding to the peptides WAAQQFFWDRIXDHDHAFSRNK (SEQ ID NO: 170) and SEVRTAVLEISGXEQAIVAGIEGLTVLK (SEQ ID NO: 171) (X=frTet) were observed at 2,905.8 m/z (FIG. 30B, bottom) and 3,012.9 m/z (FIG. 30C, bottom), which were consistent with theoretical masses of 2,904.4 and 3,011.8 m/z. Glutamine at position 175 (Q175) was contained in the peptide STGSEFHGFPRDKYTTLQETTDR (SEQ ID NO: 172, residues 158-180, theoretical mass 2,673.3 m/z) of trypsin-digested AgUox-WT. In the mass spectrum of AgUox-WT, a peak of 2,673.3 m/z consistent with the theoretical value was detected (FIG. 30D, top). In the mass spectrum of the trypsin-digested Ag10 variant (AgUox-Q175frTet), a peptide mass shift of 11.8 m/z was observed, showing a peak at 2,775.1 m/z (FIG. 30D, bottom), which was consistent with the theory. In the case of the Q175frTet variant, a mass shift of 11.1 m/z was observed. Glutamic acid at position 196 (E196) was contained in the peptide YNTVEVDFDAVYASVR (SEQ ID NO: 173, residues 192-207, theoretical mass 1,847.9 m/z). In the mass spectrum of AgUox-WT, a peak at 1,847.2 m/z consistent with the theoretical value was detected (FIG. 30E, top). In the mass spectrum of the trypsin-digested Ag12 variant (AgUox-E196frTet), a mass shift of 11.3 m/z was observed, resulting in a peak at 1,948.5 m/z consistent with the theoretical mass (FIG. 30E, bottom). This is a result consistent with a mass shift of 11.1 m/z of the E196frTet variant, indicating that frTet is site-specifically incorporated into a specific site of the AgUox-frTet variant.

### Experimental Example 6.12 Site-Specific HAS Conjugation to AgUox-frTet

To prepare HSA-conjugated AgUox, a heterobifunctional cross-linker, TCO-MAL, was used. First, TCO-MAL was conjugated to free cysteine at position 34 (Cys34) of HSA through a Michael addition reaction. The only free cysteine (Cys34) on the HSA surface was present far from the FcRn-binding domain and thus frequently used for bioconjugation. Then, TCO-HSA was conjugated to the purified AgUox-frTet variant through an IEDDA reaction to produce an AgUox-HSA conjugate. The reaction mixture was subjected to SDS-PAGE analysis (FIG. 25), and bands of HSA-conjugated AgUox-frTet (Ag1, Ag6, Ag8, Ag10, and Ag12) variants in the CBB-stained protein gel were clearly observed in the range of 100 kDa to 120 kDa (FIG. 25). In the case of the Ag1, Ag8, and Ag12 variants, no monomeric AgUox bands were observed, indicating that AgUox and HSA were almost perfectly conjugated. In the case of the Ag6 and Ag10 variants, monomeric AgUox bands were observed within 25 to 37 kDa, indicating poor conjugation of AgUox to HSA. The tendency in HSA conjugation yields of the Ag variants except for Ag8 were similar to those in solvent accessibility (0.93, 0.51, 0.9, 0.85, and 0.92 for Ag1, Ag6, Ag8, Ag10, and Ag12, respectively). The Ag12 variant exhibited the highest HSA conjugation yield and the highest enzyme activity.

### Experimental Example 6.13 Enzyme Activity of AgUox-HSA Conjugate

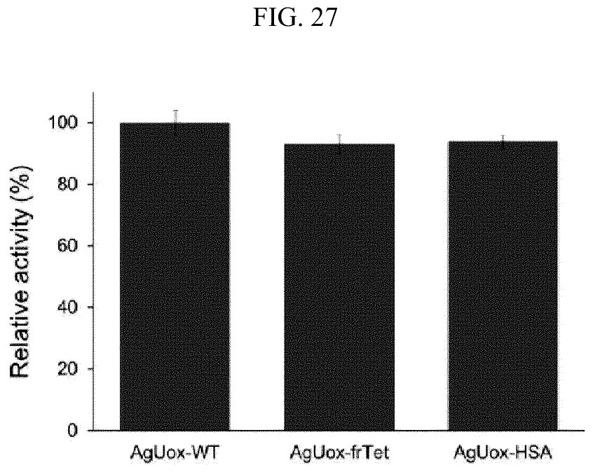

The HSA-conjugated Ag12 variant (Ag12-HSA) was purified from the reaction mixture using size-exclusion chromatography. Fractions eluted from the chromatogram were analyzed by SDS-PAGE (FIG. 26). While the two main peaks represented Ag12-HSA and unreacted HSA-TCO, respectively, the peak of Ag12 monomer was not detected, indicating a high HSA conjugation yield. Next, as a result of evaluating the enzyme activities of Ag12 and Ag12-HSA, the two exhibited enzyme activities about 93% of that of AgUox-WT (FIG. 27). Such results indicate that the Ag12 variant is suitable for site-specific HSA conjugation with maintained enzyme activity.

### Experimental Example 6.14 Pharmacokinetic Studies of AgUox-WT and Ag12-HSA

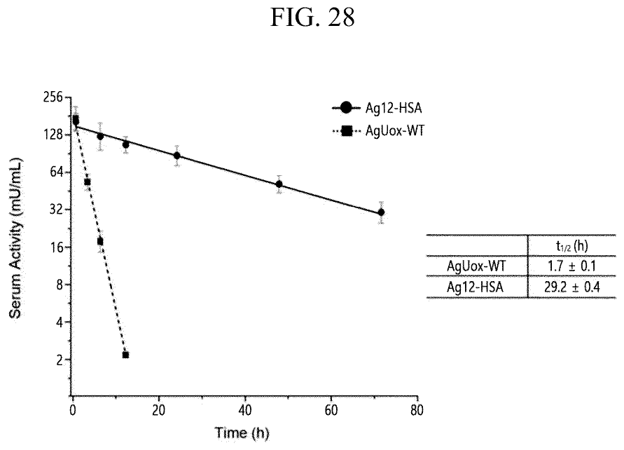

AgUox-WT and Ag12-HSA were intravenously injected into mice, and the serum half-lives of AgUox-WT and Ag12-HSA were then measured. In addition, the enzyme activities of AgUox species in serum samples were monitored. The serum half-life of AgUox-WT was about 1.7 hours (FIG. 28), which was longer than that of AfUox-WT (1.3 hours). Furthermore, the serum half-life of the Ag12-HSA conjugate was 29 hours, which was about 17 times longer than that of AgUox-WT (FIG. 28), indicating that the serum half-life of AgUox was effectively extended by HSA conjugation. Important thing is that the serum half-life of the Ag12-HSA conjugate was longer than that of AfUox-HSA (21 hours). Considering that both Ag12-HSA and AfUox-HSA have four HSA molecules conjugated to each Uox molecule using the same linker, a difference observed in serum half-lives appears to result from a difference in thermal stability. In the case of AfUox-HSA, the serum half-life of Ag12-HSA was evaluated by measuring the enzyme activities of AgUox variants remaining in the serum. Such results illustrate how to maintain the thermal stability and enzyme activity in vivo of AgUox. In addition, these data show that the conjugation of HSA to AgUox having high thermal stability results in a significantly longer serum half-life in vivo.

### INDUSTRIAL APPLICABILITY

Disclosed herein are multimeric protein-albumin conjugates and a method of preparing the same. The multimeric protein-albumin conjugate, obtained by a technology capable of showing the advantages of albumination without affecting the activity of the multimeric protein, can be used in the field of protein therapeutics to contribute to the development of improved therapeutic agents.

1. A tetramer protein-albumin conjugate represented by [formula 1]:
T-[J₁-A-J₂-HSA]ₙ [formula 1]
wherein the T is a tetramer protein variant, J₁ is a tetramer protein-linker junction, A is an anchor, J₂ is an albumin-linker junction, and HSA is Human Serum Albumin,
wherein n is 3 or 4,
the tetramer protein variant is a tetramer that four of variant subunits are oligomerized,
each of the variant subunit comprises one 4-(1,2,3,4-tetrazine-3-yl) phenylalanines (frTet), whereby the tetramer protein variant comprises four frTet,
the tetramer protein-linker junction has a junction structure formed through Inverse Electron Demand Diels-Alder (IEDDA) reaction between a tetrazine moiety of frTet of the tetramer protein and trans-cyclooctene moiety linked to the anchor,
the tetramer protein-linker junction is represented by following, 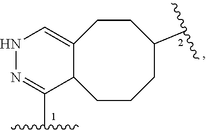 [SMILES: *C1=NNC=C2CCC(*)CCCC21]
wherein the (1) is linked to the residue of the nonnatural amino acid, and the (2) is linked to the anchor,
wherein the albumin-linker junction is a junction structure formed through a reaction between a thiol moiety of the albumin and a thiol reactive moiety of the anchor.

2. The tetramer protein-albumin conjugate of claim 1, wherein an amino acid sequence of the albumin is selected from a group consisting of SEQ ID NO: 47 to 57.

3. The tetramer protein-albumin conjugate of claim 2,
wherein the albumin-linker junction has a junction structure of which thiol group of 34th cysteine of the albumin and thiol reactive moiety of the anchor are bounded,
wherein the junction structure of the albumin-linker junction is selected from the following: 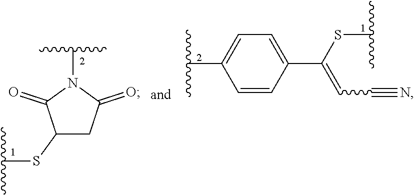
wherein, (1) is linked to the albumin, and (2) is linked to the anchor.

4. The tetramer protein-albumin conjugate of claim 1, wherein the anchor is selected from the following: 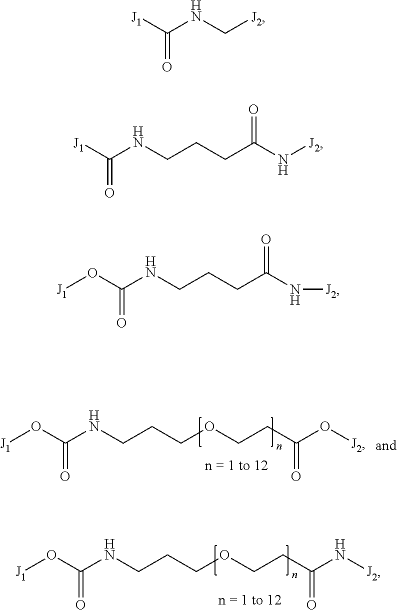
wherein J₁ is tetramer protein-linker junction, J₂ is albumin-linker junction.

5. The tetramer protein-albumin conjugate of claim 1, wherein an amino acid sequence of the variant subunit of the tetramer protein variant is selected from a group consisting of SEQ ID NOs: 4 to 17, wherein the X of the amino acid sequence is frTet.

6. The tetramer protein-albumin conjugate of claim 1, wherein an amino acid sequence of the variant subunit of the tetramer protein variant is selected from a group consisting of SEQ ID NOs: 18 to 40, and 138, wherein the X of the amino acid sequence is frTet.

7. The tetramer protein-albumin conjugate of claim 1, wherein an amino acid sequence of the variant subunit of the tetramer protein variant is selected from a group consisting of SEQ ID NOs: 41 to 45, and 139 to 158, wherein the X of the amino acid sequence is frTet.

8. A method for manufacturing a tetramer protein-albumin conjugate, the method comprises:
reacting an albumin and a linker,
wherein the linker comprises a dienophile functional group, an anchor, and a thiol reactive moiety,
wherein the dienophile functional group is a trans-cyclooctene or a derivative of trans-cyclooctene,
wherein the thiol reactive moiety is selected from a maleimide or a derivative of maleimide, and a 3-arylpropiolonitriles or a derivative of 3-arylpropiolonitriles,
wherein the thiol reactive moiety of the linker is bound with thiol moiety of albumin through reaction to form an albumin-linker conjugate; and
reacting the albumin-linker conjugate and a tetramer protein variant,
wherein, the tetramer protein variant is a tetramer that four of variant subunits are oligomerized,
wherein each of the variant subunit comprises one 4-(1,2,3,4-tetrazine-3-yl) phenylalanines (frTet), whereby the tetramer protein comprises four frTet,
wherein the tetrazine functional group of the frTet and the is bound with the dienophile functional group of the linker through Inverse Electron Demand Diels-Alder (IEDDA) reaction to form a tetramer protein-albumin conjugate,
wherein the tetramer protein-albumin conjugate is characterized in that three or more albumins are conjugated to the tetramer protein variant through the linkers.

9. The method of claim 9, wherein the linker is selected from followings:

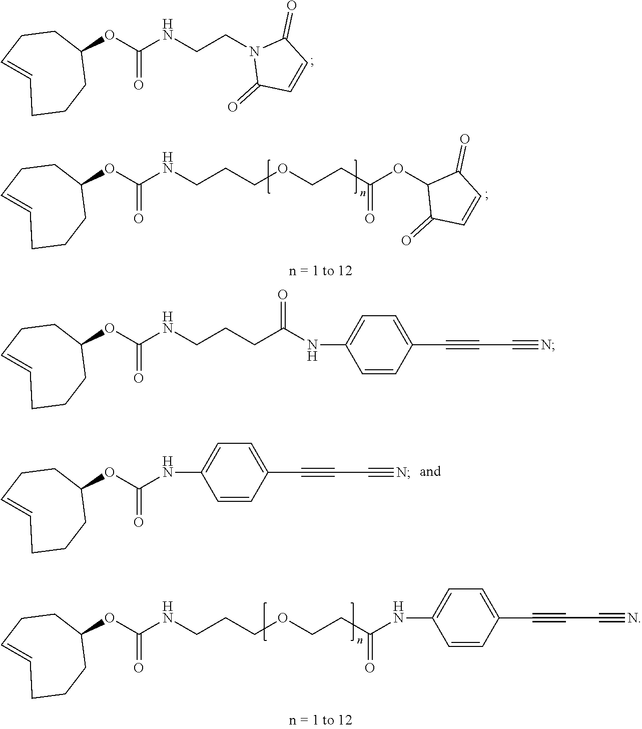

10. The method of claim 8, wherein the albumin is represented by an amino acid sequence selected from a group consisting of SEQ ID NOs: 47 to 57, wherein the thiol reactive moiety of the linker and the thiol group of 34th cysteine of the albumin are bounded through reaction.

11. The method of claim 8, wherein an amino acid sequence of the variant subunit of the tetramer protein variant is selected from a group consisting of SEQ ID NOs: 4 to 17, wherein the X of the amino acid sequence is frTet.

12. The method of claim 8, wherein an amino acid sequence of the variant subunit of the tetramer protein variant is selected from a group consisting of SEQ ID NOs: 18 to 40, and 138, wherein the X of the amino acid sequence is frTet.

13. The method of claim 8, wherein an amino acid sequence of the variant subunit of the tetramer protein variant is selected from a group consisting of SEQ ID NOs: 41 to 45, and 139 to 158, wherein the X of the amino acid sequence is frTet.

14. A method for manufacturing a tetramer protein-albumin conjugate, the method comprises:
disrupting a cell,
wherein, the cell comprises a tetramer protein variant,
wherein the tetramer protein variant comprises at least one 4-(1,2,3,4-tetrazine-3-yl) phenylalanines (frTet);
adding an albumin-linker conjugate to the cell disruption product,
wherein, the albumin-linker conjugate comprises a trans-cyclooctene or a derivative of trans-cyclooctene as a dienophile functional group,
wherein the tetrazine functional group of the frTet and the is bound with the dienophile functional group of the linker through Inverse Electron Demand Diels-Alder (IEDDA) reaction to form a multimeric protein-albumin conjugate; and
obtaining the multimeric protein-albumin conjugate.

15. The method of claim 14, wherein the method further comprises pretreatment process before adding an albumin-linker conjugate to the cell disruption product, wherein the cell disruption product is generated as a result of the pretreatment process.

16. The method of claim 14, wherein the albumin-linker conjugate is represented by [formula 2]:
I-A-J-HSA [formula 2]
wherein, the I is the dienophile functional group, selected from followings, 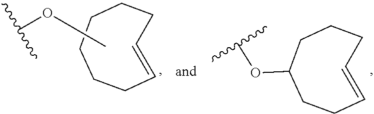
wherein the A is an anchor, selected from followings, 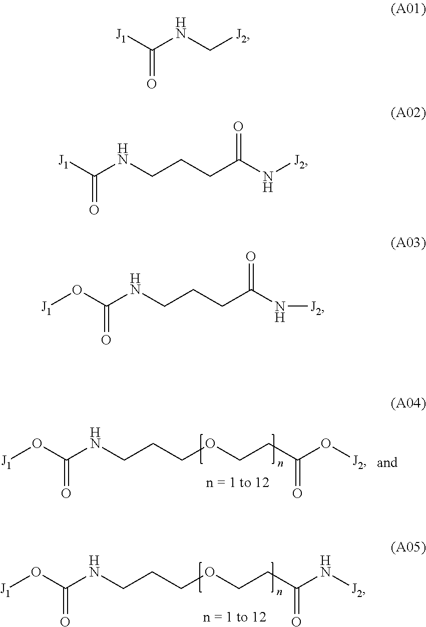
wherein the J is linker-albumin junction, selected from followings, 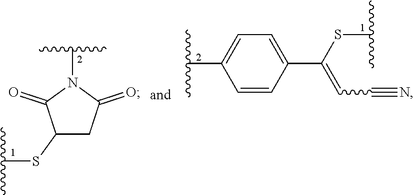 [SMILES: *SC1CC(=O)N(*)C1=O ; *SC(=CC#N)c1ccc(*)cc1]
wherein the (1) is linked to an albumin, and the (2) is linked to the anchor of the linker,
wherein the HSA is the albumin represented by a sequence selected from a group consisting of SEQ ID NOs: 47 to 57.

17. The method of claim 14, wherein the cell further comprises at least one of the followings:
pDule_C11 disclosed in Yang et.al (Temporal Control of Efficient In Vivo Bioconjugation Using a Genetically Encoded Tetrazine-Mediated Inverse-Electron-Demand Diels?Alder Reaction, Bioconjugate Chemistry, 2020, 2456-2464); tyrosyl-tRNA synthase derived from Methanococcus jannaschii (MjTyrRS); and suppressor tRNA derived from Methanococcus jannaschii (MjtRNATyrCUA).


SEQUENCE LISTING (173 sequences)

SEQ ID NO: 1 - protein, 302 aa, Artificial Sequence
  Uricase subunit, WT, Arth.Uox
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 2 - protein, 327 aa, Artificial Sequence
  Asparaginase subunit, WT
MADKLPNIVILATGGTIAGSAATGTQTTGYKAGALGVDTLINAVPEVKKLANVKGEQFSNMASENMTGDVVLKLSQRVNELLARDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 3 - protein, 397 aa, Artificial Sequence
  Methioninase subunit, WT
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 4 - protein, 302 aa, Artificial Sequence
  Uricase subunit Variant, D80
  misc_feature (80)..(80): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARXGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 5 - protein, 302 aa, Artificial Sequence
  Uricase subunit Variant, F82
  misc_feature (82)..(82): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGXATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 6 - protein, 302 aa, Artificial Sequence
  Uricase subunit Variant, F100
  misc_feature (100)..(100): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGXDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 7 - protein, 302 aa, Artificial Sequence
  Uricase subunit Variant, D101
  misc_feature (101)..(101): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFXWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 8 - protein, 302 aa, Artificial Sequence
  Uricase subunit Variant, F114
  misc_feature (114)..(114): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFXWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 9 - protein, 302 aa, Artificial Sequence
  Uricase subunit Variant, N119
  misc_feature (119)..(119): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRIXDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 10 - protein, 302 aa, Artificial Sequence
  Uricase subunit Variant, D120
  misc_feature (120)..(120): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINXHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 11 - protein, 302 aa, Artificial Sequence
  Uricase subunit Variant, S142
  misc_feature (142)..(142): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGXEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 12 - protein, 302 aa, Artificial Sequence
  Uricase subunit Variant, E143
  misc_feature (143)..(143): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSXQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 13 - protein, 302 aa, Artificial Sequence
  Uricase subunit Variant, Q175
  misc_feature (175)..(175): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLXETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 14 - protein, 302 aa, Artificial Sequence
  Uricase subunit Variant, B195
  misc_feature (195)..(195): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTXEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 15 - protein, 302 aa, Artificial Sequence
  Uricase subunit Variant, E196
  misc_feature (196)..(196): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVXVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 16 - protein, 302 aa, Artificial Sequence
  Uricase subunit Variant, H216
  misc_feature (218)..(218): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETXSLALQQTMYEMGRAVIETHPEIDEIKMSLPNKHHFLVDLQPFGQDNPNEVFYAADRPYGLIEATIQREGSRADHPIWSNIAGFC

SEQ ID NO: 17 - protein, 302 aa, Artificial Sequence
  Uricase subunit Variant, P238
  misc_feature (238)..(238): Xaa is an unnatural amino acid comprising dien functional group such as tetrazine functional group, triazine functional group, or derivatives thereof.
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHXEIDEIKMSLPNKHHFLVDLQPFGQDNPNEVFYAADRPYGLIEATIQREGSRADHPIWSNIAGFC

SEQ ID NO: 18 - protein, 328 aa, Artificial Sequence
  Asparaginase subunit variant, E80
  misc_feature (80)..(80): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNXLLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 19 - protein, 328 aa, Artificial Sequence
  Asparaginase subunit variant, A83
  misc_feature (83)..(83): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLXRDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 20 - protein, 328 aa, Artificial Sequence
  Asparaginase subunit variant, D85
  misc_feature (85)..(85): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARXDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 21 - protein, 328 aa, Artificial Sequence
  Asparaginase subunit variant, D86
  misc_feature (86)..(86): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDXVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 22 - protein, 328 aa, Artificial Sequence
  Asparaginase subunit variant, D113
  misc_feature (113)..(113): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSXKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 23 - protein, 328 aa, Artificial Sequence
  Asparaginase subunit variant, K146
  misc_feature (146)..(146): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDXQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 24 - protein, 328 aa, Artificial Sequence
  Asparaginase subunit variant, Y185
  misc_feature (185)..(185): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGXLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 25 - protein, 328 aa, Artificial Sequence
  Asparaginase subunit variant, H204
  misc_feature (204)..(204): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLXTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 26 - protein, 328 aa, Artificial Sequence
  Asparaginase subunit variant, R207
  misc_feature (207)..(207): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTXSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 27 - protein, 328 aa, Artificial Sequence
  Asparaginase subunit variant, Y235
  misc_feature (235)..(235): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLXDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 28 - protein, 328 aa, Artificial Sequence
  Asparaginase subunit variant, Q240
  misc_feature (240)..(240): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIXHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 29 - protein, 328 aa, Artificial Sequence
  Asparaginase subunit variant, G242
  misc_feature (242)..(242): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHXVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 30 - protein, 328 aa, Artificial Sequence
  Asparaginase subunit variant, S255
  misc_feature (255)..(255): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGXVSVRGIAGMRKAMEKGVVVIRSTRTGNGIVPPDEELPGLVSDSLNPAHARILLMLALTRTSDPKVIQEYFHTY

SEQ ID NO: 31 - protein, 328 aa, Artificial Sequence
  Asparaginase subunit variant, S257
  misc_feature (257)..(257): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVXVRGIAGMRKAMEKGVVVIRSTRTGNGIVPPDEELPGLVSDSLNPAHARILLMLALTRTSDPKVIQEYFHTY

SEQ ID NO: 32 - protein, 328 aa, Artificial Sequence
  Asparaginase subunit variant, R259
  misc_feature (259)..(259): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVXGIAGMRKAMEKGVVVIRSTRTGNGIVPPDEELPGLVSDSLNPAHARILLMLALTRTSDPKVIQEYFHTY

SEQ ID NO: 33 - protein, 328 aa, Artificial Sequence
  Asparaginase subunit variant, E269
  misc_feature (269)..(269): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMXKGVVVIRSTRTGNGIVPPDEELPGLVSDSLNPAHARILLMLALTRTSDPKVIQEYFHTY

SEQ ID NO: 34 - protein, 328 aa, Artificial Sequence
  Asparaginase subunit variant, K270
  misc_feature (270)..(270): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEXGVVVIRSTRTGNGIVPPDEELPGLVSDSLNPAHARILLMLALTRTSDPKVIQEYFHTY

SEQ ID NO: 35 - protein, 328 aa, Artificial Sequence
  Asparaginase subunit variant, P287
  misc_feature (287)..(287): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPXDEELPGLVSDSLNPAHARILLMLALTRTSDPKVIQEYFHTY

SEQ ID NO: 36 - protein, 328 aa, Artificial Sequence
  Asparaginase subunit variant, D288
  misc_feature (288)..(288): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPXEELPGLVSDSLNPAHARILLMLALTRTSDPKVIQEYFHTY

SEQ ID NO: 37 - protein, 328 aa, Artificial Sequence
  Asparaginase subunit variant, E289
  misc_feature (289)..(289): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDXELPGLVSDSLNPAHARILLMLALTRTSDPKVIQEYFHTY

SEQ ID NO: 38 - protein, 328 aa, Artificial Sequence
  Asparaginase subunit variant, S316
  misc_feature (316)..(316): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTXDPKVIQEYFHTY

SEQ ID NO: 39 - protein, 328 aa, Artificial Sequence
  Asparaginase subunit variant, K319
  misc_feature (319)..(319): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPXVIQEYFHTY

SEQ ID NO: 40 - protein, 328 aa, Artificial Sequence
  Asparaginase subunit variant, E323
  misc_feature (323)..(323): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQXYFHTY

SEQ ID NO: 41 - protein, 397 aa, Artificial Sequence
  Methioninase subunit variant, A144
  misc_feature (144)..(144): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEXAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 42 - protein, 397 aa, Artificial Sequence
  Methioninase subunit variant, P148
  misc_feature (148)..(148): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTXATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 43 - protein, 397 aa, Artificial Sequence
  Methioninase subunit variant, K177
  misc_feature (177)..(177): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARXHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 44 - protein, 397 aa, Artificial Sequence
  Methioninase subunit variant, P283
  misc_feature (283)..(283): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQXQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 45 - protein, 397 aa, Artificial Sequence
  Methioninase subunit variant, T300
  misc_feature (300)..(300): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYXLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 46 - protein, 585 aa, Artificial Sequence [sequence omitted]

SEQ ID NO: 47 - protein, 585 aa, Artificial Sequence
  HSA variant (V418M)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQMSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 48 - protein, 585 aa, Artificial Sequence
  HSA variant (T420A)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSAPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 49 - protein, 585 aa, Artificial Sequence
  HSA variant (E505R)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNARTFTFHADICTLSEKERQIKKQTALVELVKHKPKATKEQLKAVMDDFAAFVEKCCKADDKETCFAEEGKKLVAASQAALGL

SEQ ID NO: 50 - protein, 585 aa, Artificial Sequence
  HSA variant (E505G)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAVMDDFAAFVEKCCKADDKETCFAEEGKKLVAASQAALGL

SEQ ID NO: 51 - protein, 585 aa, Artificial Sequence
  HSA variant (V547A)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAAMDDFAAFVEKCCKADDKETCFAEEGKKLVAASQAALGL

SEQ ID NO: 52 - protein, 585 aa, Artificial Sequence
  HSA variant (K573Y)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGYKLVAASQAALGL

SEQ ID NO: 53 - protein, 585 aa, Artificial Sequence
  HSA variant (V424I)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLIEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 54 - protein, 585 aa, Artificial Sequence
  HSA variant (N429D)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRDLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 55 - protein, 585 aa, Artificial Sequence
  HSA variant (A449V)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCVEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 56 - protein, 585 aa, Artificial Sequence
  HSA variant (T467M)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKMPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 57 - protein, 585 aa, Artificial Sequence
  HSA variant (A552T)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFTAFVEKCCKADDKETCFAEEGKKLVAASQAALGL

SEQ ID NO: 58 - DNA, 909 bp, Artificial Sequence
  Uricase subunit variant encoding DNA, D80
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgttagggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 59 - DNA, 909 bp, Artificial Sequence
  Uricase subunit variant encoding DNA, F82
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttaggcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 60 - DNA, 909 bp, Artificial Sequence
  Uricase subunit variant encoding DNA, F100
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttaggattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 61 - DNA, 909 bp, Artificial Sequence
  Uricase subunit variant encoding DNA, D101
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggtttttagtgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 62 - DNA, 909 bp, Artificial Sequence
  Uricase subunit variant encoding DNA, F114
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagttttagtgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 63 - DNA, 909 bp, Artificial Sequence
  Uricase subunit variant encoding DNA, N119
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtatttaggatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 64 - DNA, 909 bp, Artificial Sequence
  Uricase subunit variant encoding DNA, D120
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaattagcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 65 - DNA, 909 bp, Artificial Sequence
  Uricase subunit variant encoding DNA, S142
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggttaggaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 66 - DNA, 909 bp, Artificial Sequence
  Uricase subunit variant encoding DNA, E143
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagctagcaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 67 - DNA, 909 bp, Artificial Sequence
  Uricase subunit variant encoding DNA, Q175
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgtaggaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 68 - DNA, 909 bp, Artificial Sequence
  Uricase subunit variant encoding DNA, B195
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataatacctaggaagttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 69 - DNA, 909 bp, Artificial Sequence
  Uricase subunit variant encoding DNA, E196
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgtttaggttgattttgatgcggtttatgcaagcgttcgtggtctgctgctgaaagcatttgcagaaacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 70 - DNA, 909 bp, Artificial Sequence
  Uricase subunit variant encoding DNA, H216
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcatagacccatagcctggcactgcagcagacaatgtatgaaatgggtcgtgcagttattgaaacccatccggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 71 - DNA, 909 bp, Artificial Sequence
  Uricase subunit variant encoding DNA, P238
atgaccgcaaccgcagaaaccagcaccggcaccaaagttgttctgggtcagaatcagtatggtaaagcagaagttcgtctggttaaagttacccgtaataccgcacgtcatgaaattcaggatctgaatgttaccagccagctgcgtggtgattttgaagcagcacataccgcaggcgataatgcacatgttgttgcaaccgatacacagaaaaacaccgtttatgcatttgcccgtgatggttttgcaaccaccgaagaatttctgctgcgtctgggtaaacatttcaccgaaggttttgattgggttaccggtggtcgttgggcagcacagcagtttttctgggatcgtattaatgatcacgatcatgcctttagccgcaataaaagcgaagtgcgtaccgcagttctggaaattagcggtagcgaacaggcaattgttgcaggtattgaaggtctgaccgttctgaaaagcaccggtagcgagtttcatggttttccgcgtgataaatacaccacactgcaagaaaccaccgatcgtattctggcaaccgatgttagcgcacgttggcgttataataccgttgaagttgattttgatgcggtttatgcaagcgttcgtggtctgctgctgaaagcatttgcagaaacccatagcctggcactgcagcagacaatgtatgaaatgggtcgtgcagttattgaaacccattaggaaattgatgagatcaaaatgagcctgccgaacaaacatcattttctggttgatctgcagccgtttggtcaggataatccgaatgaagtgttttatgcagcagatcgtccgtatggtctgattgaagcaaccattcagcgtgaaggtagccgtgcagatcatccgatttggagtaatattgcaggtttttgctaa

SEQ ID NO: 72 - DNA, 31 bp, Artificial Sequence
  Uricase, 80AUG, Forward primer
cgtggcgaaaccctaacgcgcgaacgcgtac

SEQ ID NO: 73 - DNA, 32 bp, Artificial Sequence
  Uricase, 82AUG, Forward primer
ttcagtcgtggcctaaccatcacgcgcgaacg

SEQ ID NO: 74 - DNA, 36 bp, Artificial Sequence
  Uricase, 100AUG, Forward primer
ccaccggtaacccagtcctagccttcggtaaagtgt

SEQ ID NO: 75 - DNA, 41 bp, Artificial Sequence
  Uricase, 101AUG, Forward primer
caccggtaacccactaaaagccttcggtaaagtgtttgccc

SEQ ID NO: 76 - DNA, 31 bp, Artificial Sequence
  Uricase, 114AUG, Forward primer
tacggtcccactagaactgctgggcagccca

SEQ ID NO: 77 - DNA, 35 bp, Artificial Sequence
  Uricase, 119AUG, Forward primer
atgcatggtcgtggtcctagatacggtcccagaag

SEQ ID NO: 78 - DNA, 37 bp, Artificial Sequence
  Uricase, 120AUG, Forward primer
gagaatgcatggtcgtgctagttgatacggtcccaga

SEQ ID NO: 79 - DNA, 40 bp, Artificial Sequence
  Uricase, 142AUG, Forward primer
cagctacgatcgcctgttcctaaccagagatttccagtac

SEQ ID NO: 80 - DNA, 39 bp, Artificial Sequence
  Uricase, 143AUG, Forward primer
gccagctacgatcgcctgctaagaaccagagatttccag

SEQ ID NO: 81 - DNA, 35 bp, Artificial Sequence
  Uricase, 175AUG, Forward primer
acggtcggtggtttcctacagcgtggtatatttat

SEQ ID NO: 82 - DNA, 41 bp, Artificial Sequence
  Uricase, 195AUG, Forward primer
cagcgtcgaagtccacttcctaggtgttgtaacgccaacgg

SEQ ID NO: 83 - DNA, 37 bp, Artificial Sequence
  Uricase, 196AUG, Forward primer
gtcgaagtccacctatacggtgttgtaacgccaacgg

SEQ ID NO: 84 - DNA, 38 bp, Artificial Sequence
  Uricase, 216AUG, Forward primer
cagggccagggactaagtttctgcgaatgctttcagca

SEQ ID NO: 85 - DNA, 40 bp, Artificial Sequence
  Uricase, 238AUG, Forward primer
cttgatttcgtcaatttcctagtgggtctcgataaccgcg

SEQ ID NO: 86 - DNA, 31 bp, Artificial Sequence
  Uricase, 80AUG, Reverse primer
gtacgcgttcgcgcgttagggtttcgccacg

SEQ ID NO: 87 - DNA, 32 bp, Artificial Sequence
  Uricase, 82AUG, Reverse primer
cgttcgcgcgtgatggttaggccacgactgaa

SEQ ID NO: 88 - DNA, 36 bp, Artificial Sequence
  Uricase, 100AUG, Reverse primer
acactttaccgaaggctaggactgggttaccggtgg

SEQ ID NO: 89 - DNA, 41 bp, Artificial Sequence
  Uricase, 101AUG, Reverse primer
gggcaaacactttaccgaaggcttttagtgggttaccggtg

SEQ ID NO: 90 - DNA, 31 bp, Artificial Sequence
  Uricase, 114AUG, Reverse primer
tgggctgcccagcagttctagtgggaccgta

SEQ ID NO: 91 - DNA, 35 bp, Artificial Sequence
  Uricase, 119AUG, Reverse primer
cttctgggaccgtatctaggaccacgaccatgcat

SEQ ID NO: 92 - DNA, 37 bp, Artificial Sequence
  Uricase, 120AUG, Reverse primer
tctgggaccgtatcaactagcacgaccatgcattctc

SEQ ID NO: 93 - DNA, 40 bp, Artificial Sequence
  Uricase, 142AUG, Reverse primer
gtactggaaatctctggttaggaacaggcgatcgtagctg

SEQ ID NO: 94 - DNA, 39 bp, Artificial Sequence
  Uricase, 143AUG, Reverse primer
ctggaaatctctggttcttagcaggcgatcgtagctggc

SEQ ID NO: 95 - DNA, 35 bp, Artificial Sequence
  Uricase, 175AUG, Reverse primer
ataaatataccacgctgtaggaaaccaccgaccgt

SEQ ID NO: 96 - DNA, 41 bp, Artificial Sequence
  Uricase, 195AUG, Reverse primer
ccgttggcgttacaacacctaggaagtggacttcgacgctg

SEQ ID NO: 97 - DNA, 37 bp, Artificial Sequence
  Uricase, 196AUG, Reverse primer
ccgttggcgttacaacaccgtataggtggacttcgac

SEQ ID NO: 98 - DNA, 38 bp, Artificial Sequence
  Uricase, 216AUG, Reverse primer
tgctgaaagcattcgcagaaacttagtccctggccctg

SEQ ID NO: 99 - DNA, 40 bp, Artificial Sequence
  Uricase, 238AUG, Reverse primer
cgcggttatcgagacccactaggaaattgacgaaatcaag

SEQ ID NO: 100 - DNA, 987 bp, Artificial Sequence
  Asparaginase subunit variant encoding DNA, D85
atggcagataaactgccgaacattgttattctggcaaccggtggcaccattgcaggtagcgcagcaaccggtacacagaccaccggttataaagccggtgcactgggtgttgataccctgattaatgcagttccggaagttaaaaagctggccaatgttaaaggtgaacagtttagcaatatggccagcgaaaatatgaccggtgatgttgttctgaaactgagccagcgtgttaatgaactgctggcacgttaggatgttgatggtgttgttattacccatggcaccgataccgttgaagaaagcgcatattttctgcatctgaccgtgaaaagcgataaaccggttgtttttgttgcagcaatgcgtccggcaaccgcaattagcgcagatggtccgatgaatctgctggaagcagttcgtgttgccggtgataaacagagccgtggtcgtggtgttatggttgtgctgaatgatcgtattggtagcgcacgttatattaccaaaaccaatgcaagcaccctggatacctttaaagcaaatgaagaaggttatctgggcgtcattattggcaatcgtatctattatcagaaccgcatcgacaaactgcataccacacgtagcgtttttgatgttcgtggtctgaccagcctgccgaaagtggatattctgtatggttatcaggatgatccggaatatctgtatgatgcagcaattcagcatggtgtgaaaggtattgtttatgcaggtatgggtgccggtagcgttagcgttcgtggtattgccggtatgcgtaaagcaatggaaaaaggtgttgttgtgattcgtagcacccgtaccggtaatggtattgttccgcctgatgaagaactgcctggtctggttagcgatagcctgaatccggcacatgcacgtattctgctgatgctggcactgacccgtaccagcgatccgaaagttattcaagaatatttccatacctattaa

SEQ ID NO: 101 - DNA, 987 bp, Artificial Sequence
  Asparaginase subunit variant encoding DNA, Q240
atggcagataaactgccgaacattgttattctggcaaccggtggcaccattgcaggtagcgcagcaaccggtacacagaccaccggttataaagccggtgcactgggtgttgataccctgattaatgcagttccggaagttaaaaagctggccaatgttaaaggtgaacagtttagcaatatggccagcgaaaatatgaccggtgatgttgttctgaaactgagccagcgtgttaatgaactgctggcacgtgatgatgttgatggtgttgttattacccatggcaccgataccgttgaagaaagcgcatattttctgcatctgaccgtgaaaagcgataaaccggttgtttttgttgcagcaatgcgtccggcaaccgcaattagcgcagatggtccgatgaatctgctggaagcagttcgtgttgccggtgataaacagagccgtggtcgtggtgttatggttgtgctgaatgatcgtattggtagcgcacgttatattaccaaaaccaatgcaagcaccctggatacctttaaagcaaatgaagaaggttatctgggcgtcattattggcaatcgtatctattatcagaaccgcatcgacaaactgcataccacacgtagcgtttttgatgttcgtggtctgaccagcctgccgaaagtggatattctgtatggttatcaggatgatccggaatatctgtatgatgcagcaatttagcatggtgtgaaaggtattgtttatgcaggtatgggtgccggtagcgttagcgttcgtggtattgccggtatgcgtaaagcaatggaaaaaggtgttgttgtgattcgtagcacccgtaccggtaatggtattgttccgcctgatgaagaactgcctggtctggttagcgatagcctgaatccggcacatgcacgtattctgctgatgctggcactgacccgtaccagcgatccgaaagttattcaagaatatttccatacctattaa

SEQ ID NO: 102 - DNA, 987 bp, Artificial Sequence
  Asparaginase subunit variant encoding DNA, E269
atggcagataaactgccgaacattgttattctggcaaccggtggcaccattgcaggtagcgcagcaaccggtacacagaccaccggttataaagccggtgcactgggtgttgataccctgattaatgcagttccggaagttaaaaagctggccaatgttaaaggtgaacagtttagcaatatggccagcgaaaatatgaccggtgatgttgttctgaaactgagccagcgtgttaatgaactgctggcacgtgatgatgttgatggtgttgttattacccatggcaccgataccgttgaagaaagcgcatattttctgcatctgaccgtgaaaagcgataaaccggttgtttttgttgcagcaatgcgtccggcaaccgcaattagcgcagatggtccgatgaatctgctggaagcagttcgtgttgccggtgataaacagagccgtggtcgtggtgttatggttgtgctgaatgatcgtattggtagcgcacgttatattaccaaaaccaatgcaagcaccctggatacctttaaagcaaatgaagaaggttatctgggcgtcattattggcaatcgtatctattatcagaaccgcatcgacaaactgcataccacacgtagcgtttttgatgttcgtggtctgaccagcctgccgaaagtggatattctgtatggttatcaggatgatccggaatatctgtatgatgcagcaattcagcatggtgtgaaaggtattgtttatgcaggtatgggtgccggtagcgttagcgttcgtggtattgccggtatgcgtaaagcaatgtagaaaggtgttgttgtgattcgtagcacccgtaccggtaatggtattgttccgcctgatgaagaactgcctggtctggttagcgatagcctgaatccggcacatgcacgtattctgctgatgctggcactgacccgtaccagcgatccgaaagttattcaagaatatttccatacctattaa

SEQ ID NO: 103 - DNA, 987 bp, Artificial Sequence
  Asparaginase subunit variant encoding DNA, E289
atggcagataaactgccgaacattgttattctggcaaccggtggcaccattgcaggtagcgcagcaaccggtacacagaccaccggttataaagccggtgcactgggtgttgataccctgattaatgcagttccggaagttaaaaagctggccaatgttaaaggtgaacagtttagcaatatggccagcgaaaatatgaccggtgatgttgttctgaaactgagccagcgtgttaatgaactgctggcacgtgatgatgttgatggtgttgttattacccatggcaccgataccgttgaagaaagcgcatattttctgcatctgaccgtgaaaagcgataaaccggttgtttttgttgcagcaatgcgtccggcaaccgcaattagcgcagatggtccgatgaatctgctggaagcagttcgtgttgccggtgataaacagagccgtggtcgtggtgttatggttgtgctgaatgatcgtattggtagcgcacgttatattaccaaaaccaatgcaagcaccctggatacctttaaagcaaatgaagaaggttatctgggcgtcattattggcaatcgtatctattatcagaaccgcatcgacaaactgcataccacacgtagcgtttttgatgttcgtggtctgaccagcctgccgaaagtggatattctgtatggttatcaggatgatccggaatatctgtatgatgcagcaattcagcatggtgtgaaaggtattgtttatgcaggtatgggtgccggtagcgttagcgttcgtggtattgccggtatgcgtaaagcaatggaaaaaggtgttgttgtgattcgtagcacccgtaccggtaatggtattgttccgccttaggaagaactgcctggtctggttagcgatagcctgaatccggcacatgcacgtattctgctgatgctggcactgacccgtaccagcgatccgaaagttattcaagaatatttccatacctattaa

SEQ ID NO: 104 - DNA, 987 bp, Artificial Sequence
  Asparaginase subunit variant encoding DNA, K319
atggcagataaactgccgaacattgttattctggcaaccggtggcaccattgcaggtagcgcagcaaccggtacacagaccaccggttataaagccggtgcactgggtgttgataccctgattaatgcagttccggaagttaaaaagctggccaatgttaaaggtgaacagtttagcaatatggccagcgaaaatatgaccggtgatgttgttctgaaactgagccagcgtgttaatgaactgctggcacgtgatgatgttgatggtgttgttattacccatggcaccgataccgttgaagaaagcgcatattttctgcatctgaccgtgaaaagcgataaaccggttgtttttgttgcagcaatgcgtccggcaaccgcaattagcgcagatggtccgatgaatctgctggaagcagttcgtgttgccggtgataaacagagccgtggtcgtggtgttatggttgtgctgaatgatcgtattggtagcgcacgttatattaccaaaaccaatgcaagcaccctggatacctttaaagcaaatgaagaaggttatctgggcgtcattattggcaatcgtatctattatcagaaccgcatcgacaaactgcataccacacgtagcgtttttgatgttcgtggtctgaccagcctgccgaaagtggatattctgtatggttatcaggatgatccggaatatctgtatgatgcagcaattcagcatggtgtgaaaggtattgtttatgcaggtatgggtgccggtagcgttagcgttcgtggtattgccggtatgcgtaaagcaatggaaaaaggtgttgttgtgattcgtagcacccgtaccggtaatggtattgttccgcctgatgaagaactgcctggtctggttagcgatagcctgaatccggcacatgcacgtattctgctgatgctggcactgacccgtaccagcgatccgtaggttattcaagaatatttccatacctattaa

SEQ ID NO: 105 - DNA, 42 bp, Artificial Sequence
  pTAC-Asparaginase-Infusion-F
agatatagggatccggtaccatggcagataaactgccgaaca

SEQ ID NO: 106 - DNA, 45 bp, Artificial Sequence
  pTAC-Asparaginase-Infusion-R
ctatcatcaccatcaccatcactaaggtaccaagcttggctgttt

SEQ ID NO: 107 - DNA, 35 bp, Artificial Sequence
  Asparaginase-D85UAG-F
ggcacgttaggatgttgatggtgttgttattaccc

SEQ ID NO: 108 - DNA, 31 bp, Artificial Sequence
  Asparaginase-D85UAG-R
acatcctaacgtgccagcagttcattaacac

SEQ ID NO: 109 - DNA, 35 bp, Artificial Sequence
  Asparaginase-6H-E269UAG-F
agcaatgtagaaaggtgttgttgtgattcgtagca

SEQ ID NO: 110 - DNA, 30 bp, Artificial Sequence
  Asparaginase-6H-E269UAG-R
cctttctacattgctttacgcataccggca

SEQ ID NO: 111 - DNA, 35 bp, Artificial Sequence
  Asparaginase-6H-K319UAG-F
cgatccgtaggttattcaagaatatttccatacct

SEQ ID NO: 112 - DNA, 26 bp, Artificial Sequence
  Asparaginase-6H-K319UAG-R
ataacctacggatcgctggtacgggt

SEQ ID NO: 113 - DNA, 35 bp, Artificial Sequence
  Asparaginase-Q240UAG-F
agcaatttagcatggtgtgaaaggtattgtttatg

SEQ ID NO: 114 - DNA, 33 bp, Artificial Sequence
  Asparaginase-Q240UAG-R
ccatgctaaattgctgcatcatacagatattcc

SEQ ID NO: 115 - DNA, 31 bp, Artificial Sequence
  Asparaginase-E289UAG-F
gcctgattaggaactgcctggtctggttagc

SEQ ID NO: 116 - DNA, 32 bp, Artificial Sequence
  Asparaginase-E289UAG-R
agttcctaatcaggcggaacaataccattacc

SEQ ID NO: 117 - DNA, 1197 bp, Artificial Sequence
  Methioninase subunit variant encoding DNA, A144
atgcatggcagcaataaactgcctggttttgcaacccgtgcaattcatcatggttatgatccacaggatcatggtggtgcactggttccgcctgtttatcagaccgcaacctttacctttccgaccgttgaatatggtgcagcatgttttgccggtgaacaggcaggtcacttttatagccgtattagcaatccgacactgaatctgctggaagcacgtatggcaagcctggaaggtggtgaagcaggtctggcactggcaagcggtatgggtgcaattaccagcacactgtggaccctgctgcgtccgggtgatgaagttctgctgggtaataccctgtatggttgtacctttgcatttctgcatcatggcattggtgaatttggtgttaaactgcgtcatgttgatatggccgatctgcaggcactggaataggcaatgacaccggcaacacgtgtgatttatttcgaaagtccggcaaatccgaacatgcatatggcagatattgcgggtgttgcaaaaattgcacgtaaacatggtgcaaccgttgttgtggataatacctattgtaccccgtatctgcagcgtccgctggaactgggtgcagatctggttgttcatagcgcaaccaaatatctgagcggtcatggtgatattaccgcaggtattgttgttggtagccaggcgctggttgatcgtattcgtctgcagggtctgaaagatatgaccggtgcagttctgagtccgcatgatgccgcactgctgatgcgtggtattaaaaccctgaacctgcgtatggatcgtcattgtgcaaatgcacaggttctggcagaatttctggcacgtcagccgcaggttgaactgattcattatcctggtctggcaagctttccgcagtataccctggcacgccagcagatgagccagcctggtggtatgattgcatttgaactgaaaggtggtattggtgcaggtcgtcgttttatgaatgcactgcagctgtttagccgtgcagttagcctgggtgatgccgaaagcctggcacagcatccggcaagcatgacccatagcagctatacaccggaagaacgtgcacattatggtattagcgaaggtctggttcgtctgagcgttggcctggaagatattgatgacctgctggcagatgttcagcaggccctgaaagcaagcgcataa

SEQ ID NO: 118 - DNA, 1197 bp, Artificial Sequence
  Methioninase subunit variant encoding DNA, P148
atgcatggcagcaataaactgcctggttttgcaacccgtgcaattcatcatggttatgatccacaggatcatggtggtgcactggttccgcctgtttatcagaccgcaacctttacctttccgaccgttgaatatggtgcagcatgttttgccggtgaacaggcaggtcacttttatagccgtattagcaatccgacactgaatctgctggaagcacgtatggcaagcctggaaggtggtgaagcaggtctggcactggcaagcggtatgggtgcaattaccagcacactgtggaccctgctgcgtccgggtgatgaagttctgctgggtaataccctgtatggttgtacctttgcatttctgcatcatggcattggtgaatttggtgttaaactgcgtcatgttgatatggccgatctgcaggcactggaagcagcaatgacataggcaacacgtgtgatttatttcgaaagtccggcaaatccgaacatgcatatggcagatattgcgggtgttgcaaaaattgcacgtaaacatggtgcaaccgttgttgtggataatacctattgtaccccgtatctgcagcgtccgctggaactgggtgcagatctggttgttcatagcgcaaccaaatatctgagcggtcatggtgatattaccgcaggtattgttgttggtagccaggcgctggttgatcgtattcgtctgcagggtctgaaagatatgaccggtgcagttctgagtccgcatgatgccgcactgctgatgcgtggtattaaaaccctgaacctgcgtatggatcgtcattgtgcaaatgcacaggttctggcagaatttctggcacgtcagccgcaggttgaactgattcattatcctggtctggcaagctttccgcagtataccctggcacgccagcagatgagccagcctggtggtatgattgcatttgaactgaaaggtggtattggtgcaggtcgtcgttttatgaatgcactgcagctgtttagccgtgcagttagcctgggtgatgccgaaagcctggcacagcatccggcaagcatgacccatagcagctatacaccggaagaacgtgcacattatggtattagcgaaggtctggttcgtctgagcgttggcctggaagatattgatgacctgctggcagatgttcagcaggccctgaaagcaagcgcataa

SEQ ID NO: 119 - DNA, 1197 bp, Artificial Sequence
  Methioninase subunit variant encoding DNA, K177
atgcatggcagcaataaactgcctggttttgcaacccgtgcaattcatcatggttatgatccacaggatcatggtggtgcactggttccgcctgtttatcagaccgcaacctttacctttccgaccgttgaatatggtgcagcatgttttgccggtgaacaggcaggtcacttttatagccgtattagcaatccgacactgaatctgctggaagcacgtatggcaagcctggaaggtggtgaagcaggtctggcactggcaagcggtatgggtgcaattaccagcacactgtggaccctgctgcgtccgggtgatgaagttctgctgggtaataccctgtatggttgtacctttgcatttctgcatcatggcattggtgaatttggtgttaaactgcgtcatgttgatatggccgatctgcaggcactggaagcagcaatgacaccggcaacacgtgtgatttatttcgaaagtccggcaaatccgaacatgcatatggcagatattgcgggtgttgcaaaaattgcacgttagcatggtgcaaccgttgttgtggataatacctattgtaccccgtatctgcagcgtccgctggaactgggtgcagatctggttgttcatagcgcaaccaaatatctgagcggtcatggtgatattaccgcaggtattgttgttggtagccaggcgctggttgatcgtattcgtctgcagggtctgaaagatatgaccggtgcagttctgagtccgcatgatgccgcactgctgatgcgtggtattaaaaccctgaacctgcgtatggatcgtcattgtgcaaatgcacaggttctggcagaatttctggcacgtcagccgcaggttgaactgattcattatcctggtctggcaagctttccgcagtataccctggcacgccagcagatgagccagcctggtggtatgattgcatttgaactgaaaggtggtattggtgcaggtcgtcgttttatgaatgcactgcagctgtttagccgtgcagttagcctgggtgatgccgaaagcctggcacagcatccggcaagcatgacccatagcagctatacaccggaagaacgtgcacattatggtattagcgaaggtctggttcgtctgagcgttggcctggaagatattgatgacctgctggcagatgttcagcaggccctgaaagcaagcgcataa

SEQ ID NO: 120 - DNA, 1197 bp, Artificial Sequence
  Methioninase subunit variant encoding DNA, P283
atgcatggcagcaataaactgcctggttttgcaacccgtgcaattcatcatggttatgatccacaggatcatggtggtgcactggttccgcctgtttatcagaccgcaacctttacctttccgaccgttgaatatggtgcagcatgttttgccggtgaacaggcaggtcacttttatagccgtattagcaatccgacactgaatctgctggaagcacgtatggcaagcctggaaggtggtgaagcaggtctggcactggcaagcggtatgggtgcaattaccagcacactgtggaccctgctgcgtccgggtgatgaagttctgctgggtaataccctgtatggttgtacctttgcatttctgcatcatggcattggtgaatttggtgttaaactgcgtcatgttgatatggccgatctgcaggcactggaagcagcaatgacaccggcaacacgtgtgatttatttcgaaagtccggcaaatccgaacatgcatatggcagatattgcgggtgttgcaaaaattgcacgtaaacatggtgcaaccgttgttgtggataatacctattgtaccccgtatctgcagcgtccgctggaactgggtgcagatctggttgttcatagcgcaaccaaatatctgagcggtcatggtgatattaccgcaggtattgttgttggtagccaggcgctggttgatcgtattcgtctgcagggtctgaaagatatgaccggtgcagttctgagtccgcatgatgccgcactgctgatgcgtggtattaaaaccctgaacctgcgtatggatcgtcattgtgcaaatgcacaggttctggcagaatttctggcacgtcagtagcaggttgaactgattcattatcctggtctggcaagctttccgcagtataccctggcacgccagcagatgagccagcctggtggtatgattgcatttgaactgaaaggtggtattggtgcaggtcgtcgttttatgaatgcactgcagctgtttagccgtgcagttagcctgggtgatgccgaaagcctggcacagcatccggcaagcatgacccatagcagctatacaccggaagaacgtgcacattatggtattagcgaaggtctggttcgtctgagcgttggcctggaagatattgatgacctgctggcagatgttcagcaggccctgaaagcaagcgcataa

SEQ ID NO: 121 - DNA, 1197 bp, Artificial Sequence
  Methioninase subunit variant encoding DNA, T300
atgcatggcagcaataaactgcctggttttgcaacccgtgcaattcatcatggttatgatccacaggatcatggtggtgcactggttccgcctgtttatcagaccgcaacctttacctttccgaccgttgaatatggtgcagcatgttttgccggtgaacaggcaggtcacttttatagccgtattagcaatccgacactgaatctgctggaagcacgtatggcaagcctggaaggtggtgaagcaggtctggcactggcaagcggtatgggtgcaattaccagcacactgtggaccctgctgcgtccgggtgatgaagttctgctgggtaataccctgtatggttgtacctttgcatttctgcatcatggcattggtgaatttggtgttaaactgcgtcatgttgatatggccgatctgcaggcactggaagcagcaatgacaccggcaacacgtgtgatttatttcgaaagtccggcaaatccgaacatgcatatggcagatattgcgggtgttgcaaaaattgcacgtaaacatggtgcaaccgttgttgtggataatacctattgtaccccgtatctgcagcgtccgctggaactgggtgcagatctggttgttcatagcgcaaccaaatatctgagcggtcatggtgatattaccgcaggtattgttgttggtagccaggcgctggttgatcgtattcgtctgcagggtctgaaagatatgaccggtgcagttctgagtccgcatgatgccgcactgctgatgcgtggtattaaaaccctgaacctgcgtatggatcgtcattgtgcaaatgcacaggttctggcagaatttctggcacgtcagccgcaggttgaactgattcattatcctggtctggcaagctttccgcagtattagctggcacgccagcagatgagccagcctggtggtatgattgcatttgaactgaaaggtggtattggtgcaggtcgtcgttttatgaatgcactgcagctgtttagccgtgcagttagcctgggtgatgccgaaagcctggcacagcatccggcaagcatgacccatagcagctatacaccggaagaacgtgcacattatggtattagcgaaggtctggttcgtctgagcgttggcctggaagatattgatgacctgctggcagatgttcagcaggccctgaaagcaagcgcataa

SEQ ID NO: 122 - DNA, 42 bp, Artificial Sequence; identical to SEQ ID NO: 105
  pTAC-6h-Methioninase-Infusion-F

SEQ ID NO: 123 - DNA, 45 bp, Artificial Sequence; identical to SEQ ID NO: 106
  pTAC-6h-Methioninase-Infusion-R

SEQ ID NO: 124 - DNA, 35 bp, Artificial Sequence; identical to SEQ ID NO: 107
  6h-Methioninase-D85UAG-F

SEQ ID NO: 125 - DNA, 31 bp, Artificial Sequence; identical to SEQ ID NO: 108
  6h-Methioninase-D85UAG-R

SEQ ID NO: 126 - DNA, 35 bp, Artificial Sequence; identical to SEQ ID NO: 109
  6h-Methioninase-6H-E269UAG-F

SEQ ID NO: 127 - DNA, 30 bp, Artificial Sequence; identical to SEQ ID NO: 110
  6h-Methioninase-6H-E269UAG-R

SEQ ID NO: 128 - DNA, 35 bp, Artificial Sequence; identical to SEQ ID NO: 111
  6h-Methioninase-6H-K319UAG-F

SEQ ID NO: 129 - DNA, 26 bp, Artificial Sequence
  6h-Methioninase-6H-K319UAG-R
ataacctacggatcgctggtacgggt

SEQ ID NO: 130 - DNA, 35 bp, Artificial Sequence; identical to SEQ ID NO: 113
  6h-Methioninase-Q240UAG-F

SEQ ID NO: 131 - DNA, 33 bp, Artificial Sequence; identical to SEQ ID NO: 114
  6h-Methioninase-Q240UAG-R

SEQ ID NO: 132 - DNA, 31 bp, Artificial Sequence; identical to SEQ ID NO: 115
  6h-Methioninase-E289UAG-F

SEQ ID NO: 133 - DNA, 32 bp, Artificial Sequence; identical to SEQ ID NO: 116
  6h-Methioninase-E289UAG-R

SEQ ID NO: 134 - DNA, 25 bp, Artificial Sequence
  pBAD-AgUox_F
gccgccatggtgtctgctgtgaagg

SEQ ID NO: 135 - DNA, 25 bp, Artificial Sequence
  pBAD-AgUox_R
gccgagatctttaatggtgatggtg

SEQ ID NO: 136 - DNA, 987 bp, Artificial Sequence
  Asparaginase WT encoding DNA
atggcagataaactgccgaacattgttattctggcaaccggtggcaccattgcaggtagcgcagcaaccggtacacagaccaccggttataaagccggtgcactgggtgttgataccctgattaatgcagttccggaagttaaaaagctggccaatgttaaaggtgaacagtttagcaatatggccagcgaaaatatgaccggtgatgttgttctgaaactgagccagcgtgttaatgaactgctggcacgtgatgatgttgatggtgttgttattacccatggcaccgataccgttgaagaaagcgcatattttctgcatctgaccgtgaaaagcgataaaccggttgtttttgttgcagcaatgcgtccggcaaccgcaattagcgcagatggtccgatgaatctgctggaagcagttcgtgttgccggtgataaacagagccgtggtcgtggtgttatggttgtgctgaatgatcgtattggtagcgcacgttatattaccaaaaccaatgcaagcaccctggatacctttaaagcaaatgaagaaggttatctgggcgtcattattggcaatcgtatctattatcagaaccgcatcgacaaactgcataccacacgtagcgtttttgatgttcgtggtctgaccagcctgccgaaagtggatattctgtatggttatcaggatgatccggaatatctgtatgatgcagcaattcagcatggtgtgaaaggtattgtttatgcaggtatgggtgccggtagcgttagcgttcgtggtattgccggtatgcgtaaagcaatggaaaaaggtgttgttgtgattcgtagcacccgtaccggtaatggtattgttccgcctgatgaagaactgcctggtctggttagcgatagcctgaatccggcacatgcacgtattctgctgatgctggcactgacccgtaccagcgatccgaaagttattcaagaatatttccatacctattaa

SEQ ID NO: 137 - DNA, 1197 bp, Artificial Sequence
  Methioniase WT encoding DNA
atgcatggcagcaataaactgcctggttttgcaacccgtgcaattcatcatggttatgatccacaggatcatggtggtgcactggttccgcctgtttatcagaccgcaacctttacctttccgaccgttgaatatggtgcagcatgttttgccggtgaacaggcaggtcacttttatagccgtattagcaatccgacactgaatctgctggaagcacgtatggcaagcctggaaggtggtgaagcaggtctggcactggcaagcggtatgggtgcaattaccagcacactgtggaccctgctgcgtccgggtgatgaagttctgctgggtaataccctgtatggttgtacctttgcatttctgcatcatggcattggtgaatttggtgttaaactgcgtcatgttgatatggccgatctgcaggcactggaagcagcaatgacaccggcaacacgtgtgatttatttcgaaagtccggcaaatccgaacatgcatatggcagatattgcgggtgttgcaaaaattgcacgtaaacatggtgcaaccgttgttgtggataatacctattgtaccccgtatctgcagcgtccgctggaactgggtgcagatctggttgttcatagcgcaaccaaatatctgagcggtcatggtgatattaccgcaggtattgttgttggtagccaggcgctggttgatcgtattcgtctgcagggtctgaaagatatgaccggtgcagttctgagtccgcatgatgccgcactgctgatgcgtggtattaaaaccctgaacctgcgtatggatcgtcattgtgcaaatgcacaggttctggcagaatttctggcacgtcagccgcaggttgaactgattcattatcctggtctggcaagctttccgcagtataccctggcacgccagcagatgagccagcctggtggtatgattgcatttgaactgaaaggtggtattggtgcaggtcgtcgttttatgaatgcactgcagctgtttagccgtgcagttagcctgggtgatgccgaaagcctggcacagcatccggcaagcatgacccatagcagctatacaccggaagaacgtgcacattatggtattagcgaaggtctggttcgtctgagcgttggcctggaagatattgatgacctgctggcagatgttcagcaggccctgaaagcaagcgcataa

SEQ ID NO: 138 - protein, 328 aa, Artificial Sequence
  Asparaginase subunit variant, E290
  misc_feature (290)..(290): Xaa is an unnatural amino acid comprising dien functional group such as tetrazine functional group, triazine functional group, or derivatives thereof.
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEXLPGLVSDSLNPAHARILLMLALTRTSDPKVIQEYFHTY

SEQ ID NO: 139 - protein, 397 aa, Artificial Sequence
  Methioninase subunit variant, N4
  misc_feature (4)..(4): Xaa is an unnatural amino acid comprising dien functional group such as tetrazine functional group, triazine functional group, or derivatives thereof.
HGSXKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 140 - protein, 397 aa, Artificial Sequence
  Methioninase subunit variant, L6
  misc_feature (6)..(6): Xaa is an unnatural amino acid comprising dien functional group such as tetrazine functional group, triazine functional group, or derivatives thereof.
HGSNKXPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 141 - protein, 397 aa, Artificial Sequence
  Methioninase subunit variant, H16
  misc_feature (16)..(16): Xaa is an unnatural amino acid comprising dien functional group such as tetrazine functional group, triazine functional group, or derivatives thereof.
HGSNKLPGFATRAIHXGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 142 - protein, 397 aa, Artificial Sequence
  Methioninase subunit variant, E43
  misc_feature (43)..(43): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVXYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 143 - protein, 397 aa, Artificial Sequence
  Methioninase subunit variant, A71
  misc_feature (71)..(71): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEXRMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 144 - protein, 397 aa, Artificial Sequence
  Methioninase subunit variant, S75
  misc_feature (75)..(75): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMAXLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 145 - protein, 397 aa, Artificial Sequence
  Methioninase subunit variant, G78
  misc_feature (78)..(78): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEXGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 146 - protein, 397 aa, Artificial Sequence
  Methioninase subunit variant, P102
  misc_feature (102)..(102): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRXGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 147 - protein, 397 aa, Artificial Sequence
  Methioninase subunit variant, G128
  misc_feature (128)..(128): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFXVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 148 - protein, 397 aa, Artificial Sequence
  Methioninase subunit variant, K130
  misc_feature (130)..(130): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVXLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 149 - protein, 397 aa, Artificial Sequence
  Methioninase subunit variant, A137
  misc_feature (137)..(137): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMXDLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 150 - protein, 397 aa, Artificial Sequence
  Methioninase subunit variant, Q140
  misc_feature (140)..(140): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLXALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 151 - protein, 397 aa, Artificial Sequence
  Methioninase subunit variant, R151
  misc_feature (151)..(151): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATXVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 152 - protein, 397 aa, Artificial Sequence
  Methioninase subunit variant, N162
  misc_feature (162)..(162): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPXMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 153 - protein, 397 aa, Artificial Sequence
  Methioninase subunit variant, K173
  misc_feature (173)..(173): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAXIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 154 - protein, 397 aa, Artificial Sequence
  Methioninase subunit variant, R176
  misc_feature (176)..(176): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIAXKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 155 - protein, 397 aa, Artificial Sequence
  Methioninase subunit variant, L229
  misc_feature (229)..(229): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQAXVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 156 - protein, 397 aa, Artificial Sequence
  Methioninase subunit variant, D231
  misc_feature (231)..(231): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVXRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 157 - protein, 397 aa, Artificial Sequence
  Methioninase subunit variant, R232
  misc_feature (232)..(232): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDXIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 158 - protein, 397 aa, Artificial Sequence
  Methioninase subunit variant, Q236
  misc_feature (236)..(236): Xaa is an unnatural amino acid comprising dien functional group such as tetrazine functional group, triazine functional group, or derivatives thereof.
HGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLXGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 159 - DNA, 20 bp, Artificial Sequence
  pTAC linearize-F
caagcttggctgttttggcg

SEQ ID NO: 160 - DNA, 25 bp, Artificial Sequence
  pTAC linearize-R
ctatatctccttcttaaagttaaac

SEQ ID NO: 161 - DNA, 34 bp, Artificial Sequence
  tacP-RBS-MCS-rrnBt1t2-F
aagaaggagatatagatgtctgctgtgaaggccg

SEQ ID NO: 162 - DNA, 36 bp, Artificial Sequence
  tacP-RBS-MCS-rrnBt1t2-R
aaacagccaagcttgttacagcttgctcttcagaga

SEQ ID NO: 163 - DNA, 20 bp, Artificial Sequence
  pTAC-sequencing-F
gcctagagcaagacgtttcc

SEQ ID NO: 164 - DNA, 19 bp, Artificial Sequence
  pTAC-sequencing-R
ttaatgcagctggcacgac

SEQ ID NO: 165 - DNA, 23 bp, Artificial Sequence
  Ribosome binding site
tttgtttaactttaagaaggaga

SEQ ID NO: 166 - protein, 301 aa, Artificial Sequence
  Asp.Uox Variant Subunit (W174)
  misc_feature (174)..(174): Xaa is an unnatural amino acid comprising dien functional group such as tetrazine functional group, triazine functional group, or derivatives thereof.
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETXDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 167 - protein, 20 aa, Artificial Sequence
  AgUox-WT fregment, 72-91
NTVYAFARDGFATTEEFLLR

SEQ ID NO: 168 - protein, 22 aa, Artificial Sequence
  AgUox-WT fregment, 108-129
WAAQQFFWDRINDHDHAFSRNK

SEQ ID NO: 169 - protein, 28 aa, Artificial Sequence
  AgUox-WT fregment, 130-157
SEVRTAVLEISGSEQAIVAGIEGLTVLK

SEQ ID NO: 170 - protein, 22 aa, Artificial Sequence
  AgUox variant fregment
  misc_feature (12)..(12): Xaa is an unnatural amino acid comprising dien functional group such as tetrazine functional group, triazine functional group, or derivatives thereof.
WAAQQFFWDRIXDHDHAFSRNK

SEQ ID NO: 171 - protein, 28 aa, Artificial Sequence
  AgUox variant fregment
  misc_feature (13)..(13): Xaa is an unnatural amino acid comprising dien functional group such as tetrazine functional group, triazine functional group, or derivatives thereof.
SEVRTAVLEISGXEQAIVAGIEGLTVLK

SEQ ID NO: 172 - protein, 23 aa, Artificial Sequence
  AgUox-WT fregment, 150-180
STGSEFHGFPRDKYTTLQETTDR

SEQ ID NO: 173 - protein, 16 aa, Artificial Sequence
  AgUox-WT fregment, 192-207
YNTVEVDFDAVYASVR